(19) United States
(12) Patent Application Publication
(10) Pub. No.: US 2025/0179009 A1
(43) Pub. Date: Jun. 5, 2025

(54) ACETONITRILE RECOVERY PROCESS

(71) Applicant: Novartis AG, Basel (CH)

(72) Inventors: Christian FLEURY, Delémont (CH); Bertrand GUELAT, Courfaivre (CH); Ruairí Ó MEADHRA, Sierentz (FR); Francesco VENTURONI, Saint-Louis (FR)

(21) Appl. No.: 18/845,258

(22) PCT Filed: Mar. 10, 2023
(86) PCT No.: PCT/IB2023/052332

§ 371 Date: Sep. 9, 2024

(30) Foreign Application Priority Data
Mar. 11, 2022 (EP) 22161657.6

(51) Int. Cl. C07C 253/34

(52) U.S. Cl. CPC C07C 253/34

(57) ABSTRACT

The present disclosure relates to improved processes to recover acetonitrile from aqueous and organic waste streams, particularly those generated during oligonucleotide manufacturing processes.

### FIELD OF THE DISCLOSURE

The present disclosure is directed to the recovery and/or purification of acetonitrile from waste solvent streams, particularly those generated during oligonucleotide manufacturing processes. The acetonitrile produced by the processes of the present disclosure is particularly suitable for use in the manufacture of oligonucleotides, e.g., chemically modified oligonucleotides, such as those used in therapy.

### BACKGROUND OF THE DISCLOSURE

The relative proportions of the components of the waste solvent acetonitrile can vary over a wide range depending on various conditions. Numerous chemical processes utilize acetonitrile as a solvent or wash, resulting in the generation of low grade acetonitrile waste streams. When these processes are conducted on a manufacturing scale, the volume of low grade acetonitrile waste stream produced can be substantial.

With 12 oligonucleotide drugs reaching the market to date and hundreds more in clinical trials and preclinical development, the current state of the art in oligonucleotide production poses a waste and cost burden to manufacturers. Legacy technologies make use of large volumes of hazardous reagents and solvents, as well as energy-intensive processes in synthesis, purification, and isolation. In 2016, the American Chemical Society (ACS) Green Chemistry Institute Pharmaceutical Roundtable (GCIPR) identified the development of greener processes for oligonucleotide Active Pharmaceutical Ingredients (APIs) as a critical unmet need. As a result, the Roundtable formed a focus team with the remit of identifying green chemistry and engineering improvements that would make oligonucleotide production more sustainable (Andrews et al. J. Org. Chem. 2021, 86, 49-61). For example, a typical solid-phase oligonucleotide synthesis process will require 3000 kg of acetonitrile to produce 1 kg of an oligonucleotide-based active pharmaceutical ingredient (API), hence resulting in the formation of a large amount of contaminated acetonitrile waste. There are therefore strong economic, environmental and supply reasons to regenerate the contaminated acetonitrile for re-use in the process.

Typically, solvent waste from oligonucleotide manufacture acetonitrile contains up to 90% acetonitrile and varying amounts of acrylonitrile, toluene and ethanol, amongst others, which are difficult to separate from acetonitrile, in particular on a manufacturing scale. Tight constraints are imposed on the quality of fresh acetonitrile used in the process. It must have an assay purity above 99.90% and preferably contain less than 30 ppm water such that the quality of the product oligonucleotide and oligonucleotide manufacturing process is maintained.

There exist prior methods of purifying acetonitrile from acetonitrile waste solvent. However, none of the prior art methods contemplate unique oligonucleotide feedstock streams that comprise ethanol, toluene and N-containing impurities and/or the challenges of separations involving said impurities from oligonucleotide manufacturing processes, which take account of both the organic and aqueous waste streams. Furthermore, the prior art methods are not suitable to recover acetonitrile from acetonitrile waste solvent from the organic synthesis and aqueous purification steps during oligonucleotide synthesis with an acetonitrile purity above 99.90% and in high yield. Thus, the need exists for an improved process that effectively separates and/or recovers acetonitrile from organic process waste streams, particularly, from both organic and aqueous production process waste streams.

The present disclosure is therefore directed to processes by which organic waste solvent acetonitrile streams, especially in the context of oligonucleotide manufacture, can be purified to obtain acetonitrile, which is recovered and can be reused, in particular, in oligonucleotide manufacture.

The present disclosure is also directed to processes by which aqueous and organic waste solvent acetonitrile streams can be purified in the same system, to obtain purified acetonitrile which is recovered and reused, in particular, in oligonucleotide manufacture. The new processes disclosed herein are simple and straightforward to carry out and can be conducted on a batch or continuous basis. Furthermore, the processes disclosed herein do not rely on expensive stationary phases and instead relies on energy. Surprisingly, the processes disclosed herein rely on the use of water as a separating agent. Water has a destructive effect during oligonucleotide synthesis reactions. However, by incorporating water in the regeneration process as a separating agent, although counter-intuitive, has a number of benefits for design, efficiency of purification, process integration and investment. Thus, by employing a watering step whereby water is added to an acetonitrile distillate, the desired purified acetonitrile can be obtained.

The existing prior art focuses on purifying either a water-rich stream or an organic stream but the arrangement of the processes of the present disclosure allows either or both to be processed in the same system. It can also handle a large concentration of impurities without the need for expensive regeneration steps.

### SUMMARY OF DISCLOSURE

The present disclosure is based on the findings that acetonitrile can be recovered and purified, and reused during oligonucleotide manufacture according to the processes disclosed herein. The present disclosure is also based on the discovery that highly pure acetonitrile can be recovered by a continuous distillation procedure carried out in at least three zones.

The key aspect of the process is the use of water as a separating agent. Water has a destructive effect during oligonucleotides synthesis. However, the use of water in the regeneration process as a separating agent, although largely counter-intuitive, has been surprisingly found to have a number of benefits for design, efficiency of purification, process integration and investment according to the instant disclosure.

Specifically, the addition of water to the organic side of the process facilitates separation of otherwise intractable impurities by forming binary and ternary azeotropes with lower boiling points compared to acetonitrile. This eliminates the need for a multiplicity of columns to separate impurities with a range of boiling points (above and below that of acetonitrile). The formed binary and ternary azeotropes all have a boiling points lower than acetonitrile leading to a much simpler separation task. The overall boiling point differentials relative to acetonitrile also increase. From a design point of view, this requires less stages of separation and thus less capital cost.

The use of water as a separating agent also allows both aqueous and organic streams to be processed in common equipment. On first sight, both streams would be processed separately to produce two separate qualities destined for the synthesis portion of the process (water-free) and the purification portion (water-rich). Instead, a single physical process can be used to produce a single quality that can be used in either part of the process, while taking advantage of a single common purification structure. There is a further synergy using this strategy, as the water inherent in the purification part of the process can be used as the entrainer. The process can be run independently, e.g., sequentially, using either feed stream or both combined.

The use of water in this way also reduces the loss of acetonitrile from the process as the azeotropes are largely impurity and water rich with lower acetonitrile content. As an entrainer, this property is desirable.

Two principal acetonitrile waste streams are produced from the oligonucleotide manufacturing process. A water poor organic waste stream is produced from the oligonucleotide synthesis section of the process and a water rich aqueous waste is produced from a subsequent purification step. The organic waste stream contains approximately 90 wt. % acetonitrile and the aqueous acetonitrile waste stream contains approximately 11 wt. % acetonitrile. Both of these streams are sent to a process where acetonitrile is regenerated to a quality suitable for recycle directly back to the oligonucleotide synthesis process. The amount of acetonitrile that can be regenerated in this way represents between 70 and 90% of the total acetonitrile consumed in the oligonucleotide synthesis process. Further waste streams are produced from the process of the present disclosure that are very low in acetonitrile content and sent directly to waste. This will be apparent from the description of the process described herein.

The organic waste stream (105, 205, 301) contains high levels of 2,6-lutidine, acetic anhydride, dichloroacetic acid, pyridine, toluene, 1-methyl imidazole, 5-ethylthio-1H-tetrazole and lesser amounts of diethylamine, diisopropylamine, acetic acid, N,N-dimethylformamide, organic iodine and sulphur compounds, and residual oligonucleotide building blocks and trace amounts of water. It has also been found that the acetonitrile-containing waste streams may comprise further impurities, e.g., acrylonitrile, dichloroacetic acid. Acrylonitrile and/or dichloroacetic acid may be present as an additional by-product of conventional oligonucleotide production processes and has also been found to create significant problems in the separation and/or purification of acetonitrile. Conventional methods of separation and/or purification of acetonitrile provide little or no guidance relating to effective acrylonitrile separation and separation of the above mentioned impurities. As a result, the acrylonitrile may remain present in the recovered acetonitrile product of these conventional methods, which may result in poor final purity levels and low yields of oligonudeotide product.

The aqueous waste stream (101, 201, 302) comprises primarily inorganic salts, ethanol and impure oligonudeotide strands as impurities. The remainder is acetonitrile (e.g., 6 to 13 wt %, e.g., 6 to 11 wt %, e.g., 9.5 wt %, 11 wt %) and water.

Hence, there is a need to develop a process which is capable of efficiently recovering acetonitrile from oligonucleotide synthesis waste streams, wherein the acetonitrile can be recovered at a purity of 99.90% (area %) or more, e.g., when measured by gas chromatography analysis. Preferably, the acetonitrile recovered comprises less than 30 parts per million (ppm) water. The technical problem is solved by the processes of the present disclosure.

Thus, in a first aspect, there is provided a process for recovering and/or purifying acetonitrile from waste acetonitrile (105, 205, 301) generated during oligonucleotide synthesis, the process comprising the steps:
A*) introducing an organic waste feedstock (105, 205) comprising acetonitrile, a first set of organic impurities having a lower boiling temperature than acetonitrile and a second set of organic impurities having a boiling temperature greater than acetonitrile, into a distillation column (106, 206) and separating the acetonitrile and first set of organic impurities from the second set of organic impurities, the acetonitrile and first set of organic impurities being drawn as a vapour from said distillation column and condensed to produce a distillate (107, 207), the second set of organic impurities being produced as the second distillation column bottoms (108, 208);
B*) introducing the distillate (107, 207) into a watering zone (109, 209) to produce a water enriched acetonitrile stream (111, 210, 210a);
C*) introducing the water enriched acetonitrile stream (111, 210, 210a) into a second distillation column (112, 212) and separating the first set of organic impurities from the acetonitrile, the acetonitrile being produced as the second distillation column bottoms (113, 213);
such that recovered and/or purified acetonitrile is obtained.

In a second aspect, there is provided a process for recovering and/or purifying acetonitrile from waste acetonitrile (101, 105) generated during oligonudeotide synthesis, the process comprising the steps:
A1) introducing an aqueous waste feedstock (101) comprising acetonitrile, a first set of aqueous impurities having a lower boiling temperature than acetonitrile and a second set of aqueous impurities having a boiling temperature greater than acetonitrile, into a first distillation column (102) and separating the acetonitrile and first set of impurities from the second set of impurities, the acetonitrile and first set of impurities being drawn as a vapour from said first distillation column and condensed to produce a first distillate (103), the second set of impurities being produced as the first distillation column bottoms (104);
B1) introducing an organic waste feedstock (105) comprising acetonitrile, a first set of organic impurities having a lower boiling temperature than acetonitrile and a second set of organic impurities having a boiling temperature greater than acetonitrile, into a second distillation column (106) and separating the acetonitrile and first set of organic impurities from the second set of organic impurities, the acetonitrile and first set of organic impurities being drawn as a vapour from said second distillation column and condensed to produce a second distillate (107), the second set of organic impurities being produced as the second distillation column bottoms (108);
C1) introducing the second distillate (107) into a watering zone (109) and enriching with water to produce a water enriched acetonitrile stream (111);
D1) introducing the water enriched acetonitrile stream (111) into a third distillation column (112) and separating the first set of organic impurities from the acetonitrile, the acetonitrile being produced as the third distillation column bottoms (113); and
E1) feeding the first distillate (103) of step A1 and the third distillation column bottoms (113) of step D1 to a mixing zone (116) and combining therein;
such that recovered and/or purified acetonitrile is obtained.

In a third aspect, there is provided a process for recovering acetonitrile from waste acetonitrile (201, 205) generated during oligonucleotide synthesis, the process comprising the steps:
A) introducing an aqueous waste feedstock (201) comprising acetonitrile, a first set of aqueous impurities having a lower boiling temperature than acetonitrile and a second set of aqueous impurities having a boiling temperature greater than acetonitrile, into a first distillation column (202) and separating the acetonitrile and first set of impurities from the second set of impurities, the acetonitrile and first set of impurities being drawn as a vapour from said first distillation column and condensed to produce a first distillate (203), the second set of impurities being produced as the first distillation column bottoms (204);
B) introducing an organic waste feedstock (205) comprising acetonitrile, a first set of organic impurities having a lower boiling temperature than acetonitrile and a second set of organic impurities having a boiling temperature greater than acetonitrile, into a second distillation column (206) and separating the acetonitrile and first set of organic impurities from the second set of organic impurities, the acetonitrile and first set of organic impurities being drawn as a vapour from said second distillation column and condensed to produce a second distillate (207), the second set of organic impurities being produced as the second distillation column bottoms (208);
C) feeding the first distillate of step A (203) and the second distillate of step B (207) to a watering zone (209) and
C1) combining therein to produce a water enriched acetonitrile stream (210a), or
C2) combining therein and enriching with water to produce a water enriched acetonitrle stream (210); and
D) introducing the water enriched acetonitrile stream (210, 210a) into a third distillation column (212) and separating the first sets of impurities from the acetonitrile, the acetonitrile being produced as the third distillation column bottoms (213);
such that recovered and/or purified acetonitrile is obtained.

In a fourth aspect, there is provided a process for recovering and/or purifying acetonitrile from waste acetonitrile (301, 302) generated during oligonucleotide synthesis, the process comprising the steps:
A′) introducing an organic waste feedstock (301) comprising acetonitrile, a first set of organic impurities having a lower boiling temperature than acetonitrile and a second set of organic impurities having a boiling temperature greater than acetonitrile, and an aqueous waste feedstock (302) comprising acetonitrile a first set of aqueous impurities having a lower boiling temperature than acetonitrile and a second set of aqueous impurities having a boiling temperature greater than acetonitrile, into a first mixing zone (303), and combining therein, to form an acetonitrile waste feedstock comprising said impurities; or
introducing an organic waste feedstock (301) comprising acetonitrile, a first set of organic impurities having a lower boiling temperature than acetonitrile and a second set of organic impurities having a boiling temperature greater than acetonitrile, into a first mixing zone (303), and enriching with water, to produce a water enriched acetonitrile stream comprising said impurities;
B′) introducing the acetonitrile waste feedstock or water enriched acetonitrile stream into a first distillation column (304) and separating the acetonitrile and the first set of impurities having a lower boiling temperature than acetonitrile from the second set of impurities having a boiling temperature greater than acetonitrile, the acetonitrile and first set of impurities being drawn as a vapour from said first distillation column and condensed to produce a first distillate (306), the second set of impurities being produced as the first distillation column bottoms (305);
C′) optionally introducing the first distillate (306) comprising acetonitrile and low boiling impurities, into a watering zone (307) and enriching with water to produce a water enriched acetonitrile stream (307a); and
D′) introducing the water enriched acetonitrile stream (306, 307a) into a second distillation column (308) and separating the acetonitrile from the low boiling impurities, the acetonitrile being produced as the second distillation column bottoms (309);
such that recovered and/or purified acetonitrile is obtained.

It has been demonstrated that acetonitrile regenerated in this way can be used to synthesise oligonucleotides with the same efficiency as using fresh acetonitrile. The oligonucleotide produced with the recovered acetonitrile according to the process of the present disclosure is preferably a therapeutic oligonucleotide. In particular, the oligonucleotide is single stranded having at least one modified nucleotide residue, wherein the modification is selected from the group consisting of modification at the 2′ position of the sugar moiety, modification of the nucleobase, and modification of the backbone.

In a further aspect, there is provided the use of acetonitrile for oligonucleotide manufacture, wherein the acetonitrile has a purity of at least 99.90% when measured by gas chromatography, and wherein the acetonitrile has been recovered from oligonucleotide synthesis waste, in particular, according to the processes disclosed herein.

In a further aspect, there is provided a process for synthesizing an oligonucleotide, the process comprising recovering and/or purifying acetonitrile from waste acetonitrile according to the processes disclosed herein and using at least a portion of the recovered acetonitrile in a process for synthesizing an oligonucleotide and/or washing an oligonucleotide or support bound oligonucleotide.

In a further aspect, there is provided a system for purifying and/or recovering acetonitrile from waste acetonitrile (105, 205, 301) generated during an oligonucleotide manufacturing process, the system comprising:
a first distillation column (106, 206, 304) configured to receive an acetonitrile organic waste stream (105, 205, 301) and produce a first distillate (107, 207, 306) comprising acetonitrile and a first set of impurities, the first distillation column (106, 206, 304) having a condenser connected to the upper portion of the first distillation column; and
a second distillation column (112, 212, 308) configured to receive the first distillate (107, 207, 306) and separate the first set of organic impurities from the acetonitrile, and produce purified acetonitrile as the second distillation column bottoms; wherein
the system further comprises a watering zone (109, 209, 303, 307) located such that
the first distillate (107, 207, 306) is first enriched with water before reaching the second distillation column (112, 212, 308), such that purified and/or recovered acetonitrile can be obtained; or
the waste acetonitrile (301) is enriched with water before reaching the first distillation column (304), such that purified and/or recovered acetonitrile can be obtained.

In a further aspect, there is provided a system (100) for purifying and/or recovering acetonitrile from waste acetonitrile (101, 105) generated during an oligonucleotide manufacturing process, the system comprising:
a first distillation column (102) configured to receive an acetonitrile aqueous waste stream (101) and produce a first distillate (103) comprising acetonitrile and a first set of impurities, the first distillation column (102) having a condenser connected to the upper portion of the first distillation column (102);
a second distillation column (106) configured to receive an acetonitrile organic waste stream (105) and produce a second distillate (107) comprising acetonitrile and a first set of impurities, the second distillation column (106) having a condenser connected to the upper portion of the second distillation column (106); and
a third distillation column (112) configured to receive the second distillate (107) and separate the first set of organic impurities from the acetonitrile, and produce purified acetonitrile as the third distillation column bottoms; wherein
the system further comprises a watering zone (109) located such that the second distillate (107) is first enriched with water before reaching the third distillation column (112), such that purified and/or recovered acetonitrile can be obtained; and a mixing zone (116) configured to mix the first distillate (103) from the first distillation column (102) and the third distillation column bottoms (113) from the third distillation column (112).

In a further aspect, there is provided a system (200) for purifying and/or recovering a acetonitrile from waste acetonitrile (201, 205) generated during an oligonucleotide manufacturing process, the system comprising:
a first distillation column (202) configured to receive an acetonitrile aqueous waste stream (201) and produce a first distillate (203) comprising acetonitrile and a first set of impurities, the first distillation column (202) having a condenser connected to the upper portion of the first distillation column (202);
a second distillation column (206) configured to receive an acetonitrile organic waste stream (205) and produce a second distillate (207) comprising acetonitrile and a first set of impurities, the second distillation column (206) having a condenser connected to the upper portion of the second distillation column (206); and
a third distillation column (212) configured to receive the first and second distillates (203, 207) and separate the first sets of impurities from the first and second distillates, and produce purified acetonitrile as the third distillation column bottoms (213); wherein
the system further comprises a watering zone (209) located such that the first and second distillates (203, 207) are first mixed and enriched with water before reaching the third distillation column (212), such that purified and/or recovered acetonitrile can be obtained.

In a further aspect, there is provided a system (300) for purifying and/or recovering acetonitrile from waste acetonitrile (301, 302) generated during an oligonucleotide manufacturing process, the system comprising:
a first distillation column (304) configured to receive a mixed acetonitrile aqueous and organic waste stream and produce a first acetonitrile distillate (306) comprising acetonitrile and a first set of impurities, the first distillation column (304) having a condenser connected to the upper portion of the first distillation column (304), e.g., connected to an overhead stream;
a second distillation column (308) configured to receive the first acetonitrile distillate (306) and produce a second acetonitrile distillate (309) comprising an acetonitrile/water azeotrope, the second distillation column (308) having a condenser connected to the upper portion of the second distillation column (308), e.g., connected to an overhead stream; and
a third distillation column (311) configured to receive the second acetonitrile distillate (309) and separate acetonitrile from the acetonitrile/water azeotrope, and produce acetonitrile as the third distillation column bottoms (312); wherein
the system further comprises a watering zone (303, 307) located such that the first acetonitrile distillate (306) is first enriched with water (307) before reaching the second distillation column (308), and/or
the organic waste acetonitrile (301) is first enriched with water by mixing an acetonitrile aqueous and organic waste stream (303) before reaching the first distillation column (304), such that purified and/or recovered acetonitrile can be obtained.

In yet a further aspect, there is provided acetonitrile obtained by the processes disclosed herein.

The above aspects and embodiments therein can be combined. Other objects, features, advantages and aspects of the present disclosure will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosure will become readily apparent to those skilled in the art from reading the following.

### DETAILED DESCRIPTION

### Description of Figures

FIG. 1 is an exemplary flow diagram of the practice of the process in accordance with the first aspect of the present disclosure.

FIG. 2 is an exemplary flow diagram of the practice of the process in accordance with the second aspect of the present disclosure.

FIG. 3 is an exemplary flow diagram of the practice of the process in accordance with the third aspect of the present disclosure.

### DEFINITIONS

The term “and/or” means either “and” or “or” unless indicated otherwise.

As used herein, the term “bottoms” means the fraction that remains at and/or is removed from a lower portion of the distillation column.

As used herein, the term “drying zone” refers to the process steps wherein the water content of the acetonitrile stream is reduced to recover acetonitrile with a water content of less than 30 ppm e.g., less than 29 ppm water, less than 28 ppm water, less than 27 ppm water, less than 26 ppm water, less than 25 ppm water, less than 24 ppm water, less than 23 ppm water, less than 22 ppm water, less than 21 ppm water, less than 20 ppm water, less than 19 ppm water, less than 18 ppm water, less than 17 ppm water, less than 16 ppm water, less than 15 ppm water, less than 14 ppm water, less than 13 ppm water, less than 12 ppm water, less than 11 ppm water, less than 10 ppm water, less than 9 ppm water, less than 8 ppm water, less than 7 ppm water, less than 6 ppm water, less than 5 ppm water, less than 4 ppm water, less than 3 ppm water, less than 2 ppm water, less than 1 ppm water, when measured by gas chromatography. Preferably, the water content is reduced by use of a pressure-swing distillation. Water content can be determined according to standard methods in the art, e.g., Karl Fischer titration, gas chromatography.

As used herein, the gram scale is defined as 1, 2, 3, 4, 5, 6, 7, 8, 9, 10, 20, 30, 40, 50, 60, 70, 80, 90 or 100 grams or greater, for example, 150, 200, 250, 300, 350, 400, 450, 500, 550, 600, 650, 700, 750, 800, 850, 900, 950 grams.

As used herein, the kilogram scale is defined as 1 kg or more, for example, greater than or equal to 1, 2, 3, 4, 5, 6, 7, 8, 9, 10, 15, 20, 25, 30, 35, 40, 45, 50, 55, 60, 65, 70, 75, 80, 85, 90, 95 or 100 kg.

High boiling impurities or higher boiling impurities are defined as those impurities that have a boiling temperature higher than acetonitrile (81.6° C. at atmospheric pressure). Examples of high boiling impurities are diisopropylamine (boiling point: 84° C. at atmospheric pressure), water (boiling point: 100° C. at atmospheric pressure), toluene (boiling point: 110.6° C. at atmospheric pressure), pyridine (boiling point: 115° C. at atmospheric pressure), acetic acid (boiling point: 118.1° C. at atmospheric pressure), 2,6-lutidine (boiling point: 144° C. at atmospheric pressure), N,N-dimethylformamide (boiling point: 153° C. at atmospheric pressure), dichloroacetic acid (boiling point: 194° C. at atmospheric pressure), 1-methylimidazole (boiling point: 198° C. at atmospheric pressure), without being restricted thereto.

Low boiling impurities or lower boiling impurities are defined as those impurities that have a lower boiling temperature than acetonitrile (e.g., 81.6° C. at atmospheric pressure), or that form an azeotrope that has a lower boiling temperature than acetonitrile, e.g., the acetonitrile/water azeotrope (boiling point: 76° C. at atmospheric pressure). Examples of low boiling impurities are toluene/acetonitrile azeotrope (boiling point: 81.4° C. at atmospheric pressure), diethylamine (boiling point: 55.5° C. at atmospheric pressure), acetone (boiling point: 56.2° C. at atmospheric pressure), methanol (boiling point: 64.5° C. at atmospheric pressure), ethanol/water/acetonitrile azeotrope (boiling point: 72.9° C. at atmospheric pressure), acrylonitrile (boiling point: 77° C. at atmospheric pressure), ethanol (boiling point: 78.37° C. at atmospheric pressure), without being restricted thereto.

As used herein, “industrial scale” means on a scale other than laboratory scale. Thus, the waste acetonitrile, will be larger than 5 litres, 10 litres, 100 litres, 1000 litres, 2000 litres, 3000 litres, 4000 litres, and can even be larger than 5000 litres. Alternatively, the waste acetonitrile will be larger than 10 kg, 100 kg, 1000 kg, 2000 kg, 3000 kg, 4000 kg, 5000 kg, 10,000 kg, 20,000 kg and can even be larger than 30,000 kg.

As used herein, the term “modified oligonucleotide” means a nucleotide residue or oligonucleotide which contains at least one aspect of its chemistry that differs from a naturally occurring nucleotide residue or oligonucleotide. Such modifications can occur in any part of the nucleotide residue, e.g., sugar, base or phosphate. Examples of modifications of nucleotides are disclosed herein. In some embodiments, the modification is selected from: (a) a modified backbone, optionally selected from a phosphorothioate (e.g. chiral phosphorothioate) or methylphosphonate internucleotide linkage; (b) a modified nucleotide, optionally selected from 2′-O-methyl (2′-OMe), 2′-fluoro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), 2′,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), and cyclopropyl phosphonate deoxyribonucleotide; and/or (c) conjugation to a ligand, e.g., a GalNAc ligand.

As used herein, the term “mixing zone” refers to the process steps whereby one or more components, e.g., process streams, are mixed with another composition, e.g., with another process stream of the system. The mixing zone may comprise a vessel capable of mixing one or more components, e.g., process streams of the process.

As used herein, the term “oligonucleotide” means a polymer of nucleotide residues, either deoxyribonucleotides (wherein the resulting oligonucleotide is DNA), ribonucleotides (wherein the resulting oligonucleotide is RNA), or a mixture thereof. An oligonucleotide may be entirely composed of nucleotide residues as found in nature or may contain at least one nucleotide, or at least one linkage between nucleotides, that has been modified. Oligonucleotides can be single stranded or double stranded. In an embodiment, the oligonucleotide is 10 to 200 nucleotides long, optionally 20 to 30 nucleotides long, optionally 20 to 25 nucleotides long. An oligonucleotide of the present disclosure may be conjugated to another molecule, e.g., N-Acetylgalactosamine (GalNAc) or multiples thereof (GalNAc clusters). Thus, the term oligonucleotide encompasses all types of oligonucleotides of any particular length, including modified oligonucleotides, and therapeutic oligonucleotides.

As used herein, the term “overhead stream” means a stream or fraction removed from an upper portion of a distillation column.

As used herein, the term “purified”, “pure” or “substantially pure” refers to the assay purity of acetonitrile, which is at least 95% (area %) on a water-free basis, when measured by gas chromatography. In particular, “purified”, “pure” or “substantially pure” refers to the purity of acetonitrile, which is at least 96% (area %), at least 97% (area %), at least 98% (area %), at least 99% (area %) at least 99.10% (area %) at least 99.20% (area %) at least 99.30% (area %) at least 99.40% (area %) at least 99.50% (area %) at least 99.60% (area %) at least 99.70% (area %) at least 99.80% (area %) at least 99.90% (area %), 99.91% (area %), 99.92% (area %), 99.93% (area %), 99.94% (area %), 99.95% (area %), 99.96% (area %), 99.97% (area %), 99.98% (area %), 99.99% (area %) on a water-free basis, when measured by gas chromatography. A “water-free” basis indicates that the composition refers to a composition on the basis of the organic components only in the sample. The fraction of water is excluded from this measurement.

As used herein, the term “highly purified” or “highly pure” refers to the purity of acetonitrile, which is at least 99.9% (area %) and has less than 30 ppm water, when measured by gas chromatography. In particular, “highly purified” or “highly pure” refers to the purity of acetonitrile, which is at least 96% (area %), at least 97% (area %), at least 98% (area %), at least 99% (area %) at least 99.10% (area %) at least 99.20% (area %) at least 99.30% (area %) at least 99.40% (area %) at least 99.50% (area %) at least 99.60% (area %) at least 99.70% (area %) at least 99.80% (area %) at least 99.90% (area %), 99.91% (area %), 99.92% (area %), 99.93% (area %), 99.94% (area %), 99.95% (area %), 99.96% (area %), 99.97% (area %), 99.98% (area %), 99.99% (area %), and has less than 30 ppm water, e.g., less than 29 ppm water, less than 28 ppm water, less than 27 ppm water, less than 26 ppm water, less than 25 ppm water, less than 24 ppm water, less than 23 ppm water, less than 22 ppm water, less than 21 ppm water, less than 20 ppm water, less than 19 ppm water, less than 18 ppm water, less than 17 ppm water, less than 16 ppm water, less than 15 ppm water, less than 14 ppm water, less than 13 ppm water, less than 12 ppm water, less than 11 ppm water, less than 10 ppm water, less than 9 ppm water, less than 8 ppm water, less than 7 ppm water, less than 6 ppm water, less than 5 ppm water, less than 4 ppm water, less than 3 ppm water, less than 2 ppm water, less than 1 ppm water, when measured by gas chromatography. In an embodiment, the processes and systems of the present disclosure result in highly pure acetonitrile. Purity can be measured according to standard methods in the art, e.g., titration, NMR, chromatography, e.g., HPLC, GC, GC/MS.

As used herein, the term “recovered acetonitrile” refers to acetonitrile which has been purified and/or regenerated from waste acetonitrile generated during the manufacturing process, e.g., during the solid phase oligonucleotide manufacturing process.

As used herein, the term “support-bound oligonucleotide” means an oligonucleotide conjugated or bound to a support material.

The support material may be a soluble support material. The soluble support material may be selected from the group consisting of polyethylene glycol, a soluble organic polymer, DNA, a protein, a dendrimer, a polysaccharide, an oligosaccharide, and a carbohydrate. The support material may be an insoluble support material. The support material may be a solid support material. The solid support material may be selected from the group consisting of a glass bead, a polymeric bead, a fibrous support, a membrane, a streptavidin coated bead and cellulose.

A solid support may also refer to a porous or non-porous solvent insoluble material. As used herein “porous” means that the material contains pores having substantially uniform diameters (for example in the nm range). Porous materials include paper, synthetic filters etc. In such porous materials, the reaction may take place within the pores. The support can have any one of a number of shapes, such as pin, strip, plate, disk, rod, cylindrical structure, particle, including bead, and the like. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, ceramics, metals, and the like etc.; either used by themselves or in conjunction with other materials.

As used herein, the term “therapeutic oligonucleotide” means an oligonucleotide that has a therapeutic application. Such an oligonucleotide typically contains one or more modified nucleotide residues or linkages. Therapeutic oligonucleotides act via one of several different mechanisms, including, but not limited to, antisense, splice-switching or exon-skipping, immunostimulation and RNA interference (RNAi), e.g., via microRNA (miRNA) and small interfering RNA (siRNA). A therapeutic oligonucleotide may be an aptamer. Therapeutic oligonucleotides will usually, but not always, have a defined sequence. Exemplary oligonucleotides include, but are not limited to, antisense oligonucleotides (ASOs), siRNAs, miRNAs, miRNA mimics, shRNAs, aptamers, antimicroRNAs, guide molecules for CRISPR systems, DNA decoys, DNAzymes.

As used herein, the term “watering zone” refers to the process step whereby one or more process streams are mixed with water. The watering zone may comprise a vessel capable of mixing water with one or more components, e.g., process streams of the process. The water may be added directly and/or may be added as a component of another process stream, e.g., present in the acetonitrile distillate from the aqueous purification step (103, 203) or waste stream (301). Water may be added to the watering zone via any conventional means known in the art, e.g., injected into the process stream, added to a vessel via an inlet or by incorporating a static mixer.

As used herein, the term “waste acetonitrile” or“oligonucleotide synthesis waste” shall mean an acetonitrile composition comprising aqueous inorganic and organic impurities, e.g., from synthesis and washing steps, such as water, toluene, ethanol, diisopropylamine, diethylamine, acrylonitrile, imidazole, 2,6-lutidine, acetic acid, dimethylformamide, ETT, PADS, pyridine, and non-volatile salts, e.g., as an aqueous mixture. The aqueous acetonitrile waste or “aqueous impurities” emanate from the purification step of the oligonucleotide process while the organic waste or “organic impurities” emanate from the oligonucleotide synthesis section. The relative proportions of the components of the waste acetonitrile, e.g., in the organic and aqueous waste streams can vary over a wide range depending on various conditions, which may vary from one process to another depending on the oligonucleotide to be synthesised.

### Processes of the Present Disclosure

In one aspect, there is provided a process for recovering and/or purifying acetonitrile from waste acetonitrile (105, 205) generated during oligonucleotide synthesis, the process comprising the steps:
A*) introducing an organic waste feedstock (105, 205) comprising acetonitrile, a first set of organic impurities having a lower boiling temperature than acetonitrile and a second set of organic impurities having a boiling temperature greater than acetonitrile, into a distillation column (106, 206) and separating the acetonitrile and first set of organic impurities from the second set of organic impurities, the acetonitrile and first set of organic impurities being drawn as a vapour from said distillation column and condensed to produce a distillate (107, 207), the second set of organic impurities being produced as the second distillation column bottoms (108, 208);
B*) introducing the distillate (107, 207) into a watering zone (109, 209) to produce a water enriched acetonitrile stream (111, 210, 210a);
C*) introducing the water enriched acetonitrile stream (111, 210, 210a) into a second distillation column (112, 212) and separating the first set of organic impurities from the acetonitrile, the acetonitrile being produced as the second distillation column bottoms (113, 213);
such that recovered and/or purified acetonitrile is obtained.

Advantageously, the above process steps A* to C* provide an improved acetonitrile recovery and/or purification process. Such process can successfully remove high boiling hydrophobic organic impurities from acetonitrile waste streams, e.g., those produced during oligonucleotide synthesis. The acetonitrile produced according to steps A* to C* is 99.90% (area %) pure according to gas chromatography analysis. Such recovered acetonitrile can be conveniently reused directly in oligonucleotide synthesis processes.

In one aspect, there is provided a process for recovering acetonitrile from waste acetonitrile (101, 105) generated during oligonucleotide synthesis, the process comprising the steps:
A1) introducing an aqueous waste feedstock (101) comprising acetonitrile, a first set of aqueous impurities having a lower boiling temperature than acetonitrile and a second set of aqueous impurities having a boiling temperature greater than acetonitrile, into a first distillation column (102) and separating the acetonitrile and first set of impurities from the second set of impurities, the acetonitrile and first set of impurities being drawn as a vapour from said first distillation column and condensed to produce a first distillate (103), the second set of impurities being produced as the first distillation column bottoms (104);
B1) introducing an organic waste feedstock (105) comprising acetonitrile, a first set of organic impurities having a lower boiling temperature than acetonitrile and a second set of organic impurities having a boiling temperature greater than acetonitrile, into a second distillation column (106) and separating the acetonitrile and first set of organic impurities from the second set of organic impurities, the acetonitrile and first set of organic impurities being drawn as a vapour from said second distillation column and condensed to produce a second distillate (107), the second set of organic impurities being produced as the second distillation column bottoms (108);
C1) introducing the second distillate (107) into a watering zone (109) and enriching with water to produce a water enriched acetonitrile stream (111);
D1) introducing the water enriched acetonitrile stream (111) into a third distillation column (112) and separating the first set of organic impurities from the acetonitrile, the acetonitrile being produced as the third distillation column bottoms (113); and
E1) feeding the first distillate (103) of step A1 and the third distillation column bottoms (113) of step D1 to a mixing zone (116) and combining therein;
such that recovered and/or purified acetonitrile is obtained.

The distillations of steps A1 and B1 may be executed independently, e.g., one after the other in any order or ratio ranging from 0-100% or in parallel, e.g., concurrently. The steps may be started concurrently as the waste streams will be produced at the same rate from the main synthesis/purification sections of the process, although this is not essential.

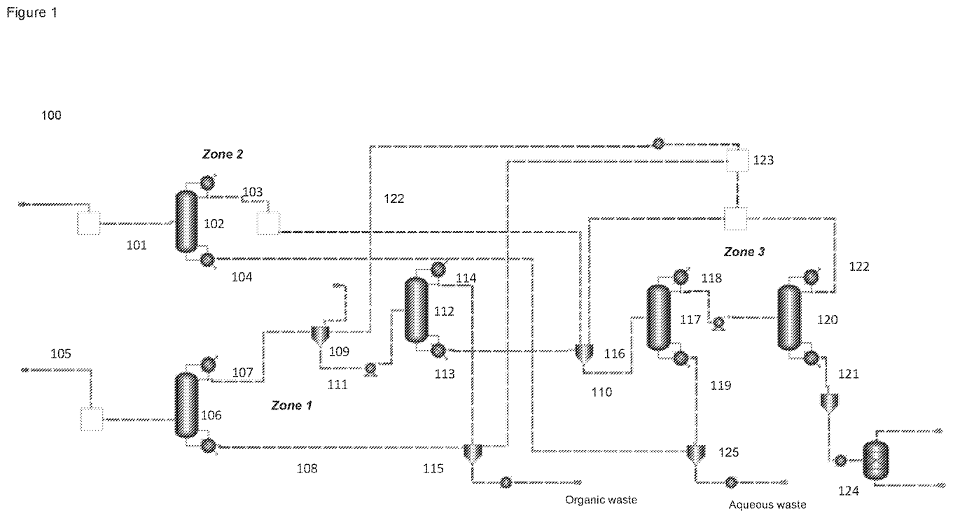

The process step A1 takes place in the so-called second distillation zone (FIG. 1, zone 2) also termed the inorganic aqueous purification zone. The aqueous acetonitrile waste (101) entering this zone is separated into a stream (103) enriched in acetonitrile and comprising some water and ethanol. The heavier boiling portion (104) comprises non-volatile salts and organic residue from the main process purification as an aqueous solution. This zone is operated to maximize the yield of acetonitrile in the form of an azeotrope while removing as much excess water as possible from the process.

In step A1, the acetonitrile fraction is drawn from the upper portion, e.g., from the top of the distillation column (102). The acetonitrile fraction leaves as a vapour and is passed through a condenser to produce a condensate (103). A portion of the condensate is then returned to the column as a reflux stream and the remainder is removed as a product. The acetonitrile fraction (103) comprises acetonitrile and light boiling impurities, such as an acetonitrile/water azeotrope.

The acetonitrile fraction may additionally comprise ethanol. Heavy aqueous impurities, e.g., having a boiling temperature greater than acetonitrile, are drawn as a distillation column bottoms (104). The heavy boiling aqueous impurities (104) comprise non-volatile salts and organic residue and is withdrawn as a bottoms product. The bottoms product (104) is considered as waste and is discarded. The bottoms product (104) or at least a portion thereof is then fed to a separate mixing zone (125), e.g., in the third distillation zone (FIG. 1, zone 3) whereby it is mixed with the aqueous bottoms product from a further processing step, e.g., from the lower pressure distillation (119) in the third distillation zone, before being discarded.

The process steps B1, C1 and D1 take place in the so-called first distillation zone (FIG. 1, zone 1), also termed the organic purification zone. The organic waste stream (105) fed to this zone contains a mixture of acetonitrile, and organic impurities, such as 2,6-lutidine, toluene, acrylonitrile and lesser amounts of pyridine, imidazole, 1-methylimidazole, diethylamine, diisopropylamine, acetic acid, 5-ethylthio-1H-tetrazole, N,N-dimethylformamide, organic iodine and sulphur compounds, and residual oligonucleotide building blocks, as well as trace amounts of water. Many of these compounds are difficult to separate from acetonitrile as they either have very similar volatilities as acetonitrile within certain concentration ranges, or they form azeotropic mixtures with acetonitrile.

The distillation step B1 comprises introducing an organic waste feedstock (105) comprising acetonitrile and the aforementioned organic impurities into a first distillation column (106), distilling said organic waste comprising acetonitrile to remove organic impurities therefrom and produce a first acetonitrile distillate (107) comprising acetonitrile and a first set of impurities comprising azeotrope formers and low boiling impurities, and a second set of impurities comprising heavy organics, e.g., having a boiling temperature greater than acetonitrile. The acetonitrile distillate along with the first set of impurities (107) is withdrawn from the upper portion, e.g., from the top of the distillation column (106). The second set of impurities is produced as the distillation column bottoms product (108). The first set of impurities generally have a lower boiling temperature relative to water (100° C. under atmospheric pressure), in particular relative to acetonitrile (82° C. under atmospheric pressure). The azeotrope formers comprise at least one of toluene, ethanol, and acrylonitrile, or mixtures thereof. The low boiling impurities comprise at least one of diethylamine, diisopropylamine, and acrylonitrile, or mixtures thereof.

The second set of impurities comprising heavy organics (108) comprises at least one of toluene, acetic acid, and a N-containing compound, or mixtures thereof. In an embodiment, the N-containing compound is at least one of 1-methylimidazole, 2,6-lutidine, dimethylformamide, pyridine, acrylonitrile, diisopropylamine, diethylamine, phenyl acetyl(disulfide), and 5-(ethylthio)-1H-tetrazole,5-(ethylthio)tetrazole, or mixtures thereof.

Step C1 comprises introducing the distillate (107) from step B1 into a watering zone (109) and enriching said distillate (107) with water to produce a water enriched acetonitrile stream (111). In order to separate azeotrope formers with acetonitrile, such as toluene, acrylonitrile or ethanol, an entrainer (e.g., water) can be added to the feed that will produce lower boiling azeotropes with these compounds relative to acetonitrile making them easier to separate from acetonitrile. To inventors' surprise, water, unexpectedly, reduces the loss of acetonitrile from the process as the azeotropes are largely impurity and water rich with lower acetonitrile content. The amount of water added relative to acetonitrile may be in excess of 0.24 w/w, but can be anywhere between 0.05 and 0.40 w/w or greater based on the amount of acetonitrile present in the stream. An amount in excess of 0.24 w/w ensures an optimal separation of toluene and acrylonitrile from acetonitrile. The amount of water added is related to the amount of acetonitrile which ensures that column operation is stable.

The lower boiling azeotropes of the water enriched acetonitrile stream (111) of step C1 comprise at least one of acetonitrile, water, toluene, ethanol, diethylamine, diisopropylamine, and acrylonitrile, or mixtures thereof. Preferably, the lower boiling azeotropes of step C1 comprise at least one of acetonitrile/water, acrylonitrile/water, acetonitrile/toluene, acetonitrile/diethylamine, acetonitrile/diisopropylamine, toluene/water, ethanol/water, toluene/water/acrylonitrile, toluene/water/acetonitrile, diisopropylamine/water/acetonitrile, diethylamine/toluene/water, or mixtures thereof.

The water enriched acetonitrile stream (111) is fed into a distillation column (112). The water enriched acetonitrile feed (111) is distilled and separated into an acetonitrile bottoms product (113) and a top fraction (114) comprising lower boiling azeotropes, the acetonitrile bottoms product (113) having a greater acetonitrile concentration than said water enriched acetonitrile stream (111) from step C1. The lower boiling azeotropes of the water enriched acetonitrile stream (111) are drawn from the upper portion, e.g., from the top of the distillation column (112). Such lower boiling azeotropic fraction (114) may be directed to organic waste. In an embodiment, at least a portion of said lower boiling azeotropic fraction (114) is first mixed, e.g., in a separate mixing zone (115), with other organic impurities, e.g., from a previous distillation step (108), before being directed to organic waste.

Advantageously, the above process steps A1 to D1 provide an improved acetonitrile recovery and/or purification process. Such process can successfully remove high boiling hydrophobic organic and inorganic aqueous impurities from acetonitrile waste streams, e.g., those produced during oligonucleotide synthesis. The amount of acetonitrile that can be regenerated in the process employing steps A1 to D1 represents between 70 and 90% of the total acetonitrile consumed in the oligonucleotide synthesis process. The acetonitrile produced according to steps A1 to D1 is 99.90% (area %) pure according to gas chromatography analysis. Such recovered acetonitrile can be conveniently reused directly in oligonucleotide synthesis processes.

In an embodiment of the present disclosure the distillation column (102) reflux ratio of step A1 is 0.25 to 2, e.g., 1. In an embodiment of the present disclosure the distillation column (106) reflux ratio of step B1 is 2 to 8, e.g., 5. In an embodiment of the present disclosure the distillation column (112) reflux ratio of step D1 is 5 to 40, e.g., 30.

The acetonitrile stream produced in step D1 may be subjected to further processing steps. Thus, in step E1 the acetonitrile bottoms product (113) from step D1 and the acetonitrile distillate (103) from step A1 may thereafter be fed to a mixing zone (116) and combined therein, to produce an acetonitrile enriched stream (110). The resultant acetonitrile enriched stream comprises purified acetonitrile, in particular, having a purity of at least 99.90% (area %) when measured by gas chromatography (GC).

The acetonitrile enriched stream may thereafter be fed to a de-watering zone (also referred to as the third distillation zone (FIG. 1, zone 3)), wherein the water content of the incoming acetonitrile stream is reduced by use of a pressure-swing distillation to recover acetonitrile with a water content of less than 30 ppm.

The acetonitrile recovered from the pressure-swing distillation step is more than 99.90% pure, e.g., when measured by gas chromatography. Preferably, the recovered acetonitrile has a purity of at least 99.90% (area %) and a water content of 30 ppm or less. Such recovered acetonitrile can be conveniently reused directly in oligonucleotide synthesis processes.

In an embodiment, the process of the present disclosure further comprises the steps:

F1) introducing the acetonitrile enriched stream (110) into a fourth distillation column (117) and, performing a distillation at below atmospheric pressure to remove water therefrom, the acetonitrile being drawn as a vapour from said fourth distillation column and condensed to produce a fourth distillate (118), and water being produced as the fourth distillation column bottoms (119); and

F2) introducing the fourth distillate (118) into a fifth distillation column (120) and, performing a second distillation at above atmospheric pressure, e.g., 5 bar, to produce lower boiling fraction (122) being drawn via the upper portion of the fifth distillation column (120) and acetonitrile being produced as the fifth distillation column bottoms (121).

The acetonitrile drawn from said column in step F2 is more than 99.90% pure when measured by GC based on the total content of the acetonitrile stream (121).

The lower boiling fraction drawn via the upper portion, e.g., from the top of the distillation column in step F1 comprises an acetonitrile composition approaching the azeotropic composition. The lower boiling impurities of step F2 comprise at least one of ethanol, water, ethanol/water azeotrope and an acetonitrile/water azeotrope, or mixtures thereof.

Thereby it is ensured that the acetonitrile stream (118) reaching the fifth distillation column (120), in which the distillation of step F2 is performed, has a reduced water content and is as rich as possible in acetonitrile. During the distillation step F2, which is performed at a higher pressure than distillation step F1, the water content increases and reaches at least 23% by weight excluding the azeotropic composition, and produces a composition that approaches the azeotropic composition at the operating pressure of the column. Hence, the remaining water can be distilled away in the form of an acetonitrile/water azeotrope. This azeotrope (122) may be recycled to the acetonitrile feedstock for the distillation of step D1, e.g., via the watering zone (109) of step C1.

In an embodiment of the present disclosure the distillation column (117) reflux ratio of step F1 is 0.05 to 1, e.g., 0.1. In an embodiment of the present disclosure the distillation column (120) reflux ratio of step F2 is 0.05 to 1, e.g., 0.25.

In a preferred embodiment, at least a portion of the acetonitrile/water azeotrope (122) is recycled back to the distillation of step D1 with a greater proportion being sent back to mixing step (116).

Thus, in an embodiment, any impurities present in the de-watering zone, e.g., from the pressure swing distillation step, e.g., in step F2, which may form light boiling azeotropes with water and acetonitrile are withdrawn from the upper portion, e.g., from the top of the column (120) and are recycled back to the first distillation zone along with any remaining acetonitrile and water. In an embodiment, the azeotropic fraction (122) from the de-watering zone, e.g., step F2, is fed back into step D1. In a further embodiment, said lighter boiling azeotropic fraction (122) is fed or recycled back into step D1 of the first distillation zone via the watering zone (109) of step C1 where the feed is enriched with water to produce lower boiling azeotropes relative to acetonitrile (111), and thereafter introduced into the third distillation column (112) in the which the distillation of step D1 is performed.

Alternatively, the lower boiling azeotropic fraction (122) is recycled directly into said mixing step (116). In an embodiment, the lower boiling azeotropic fraction (122) is recycled back into said de-watering zone, e.g., the pressure swing distillation step, e.g., steps F1 and F2, via the mixing zone (116) in which the acetonitrile feeds from steps A1 and D1 are combined. In a preferred embodiment, at least a portion of the lighter boiling azeotropic fraction (122) is recycled back to the distillation of step D1 with a greater proportion being sent back to the mixing step (116) in which the acetonitrile feeds from steps A1 and D1 are combined.

Alternatively, the lower boiling azeotropic fraction (122) from the de-watering zone, e.g., from step F2, is purged (123) and sent to organic waste (115).

In the case that the water content in the acetonitrile product stream from step F2 is not low enough, an additional stage of separation (124) can be added that could remove the residual water as an azeotrope or it could be removed by adsorption on to a stationary phase, e.g., molecular sieves.

Therefore, in a further embodiment of the disclosure, the process additionally comprises the step of feeding the acetonitrile bottoms product (121) from step F2 to a water adsorption zone (124) and contacting said acetonitrile bottoms product (121) with a water reducing adsorbent to produce acetonitrile with less than 30 ppm water that exits the adsorption zone (124). Further details and embodiments for the adsorbent(s) are described at [0033] to [0038] of WO2015126713A1.

The water reducing adsorbent may be molecular sieves. In an embodiment, the molecular sieves have a pore size less than 20 Angstroms, e.g., 3, 4 or 5 Angstroms. In a preferred embodiment, the molecular sieves have a pore size of 3 Angstroms. Since water has a size less than this and acetonitrile has a size greater than this, a pore size of 3 Angstroms is very efficient for separation. The acetonitrile leaving the adsorption zone has purity of at least 99.90% (area %), e.g., when measured by gas chromatography and a water content of below 30 ppm.

In another aspect, there is provided a process for recovering acetonitrile from waste acetonitrile (201, 205) generated during oligonucleotide synthesis, the process comprising the steps:
A) introducing an aqueous waste feedstock (201) comprising acetonitrile, a first set of aqueous impurities having a lower boiling temperature than acetonitrile and a second set of aqueous impurities having a boiling temperature greater than acetonitrile, into a first distillation column (202) and separating the acetonitrile and first set of impurities from the second set of impurities, the acetonitrile and first set of impurities being drawn as a vapour from said first distillation column and condensed to produce a first distillate (203), the second set of impurities being produced as the first distillation column bottoms (204);
B) introducing an organic waste feedstock (205) comprising acetonitrile, a first set of organic impurities having a lower boiling temperature than acetonitrile and a second set of organic impurities having a boiling temperature greater than acetonitrile, into a second distillation column (206) and separating the acetonitrile and first set of organic impurities from the second set of organic impurities, the acetonitrile and first set of organic impurities being drawn as a vapour from said second distillation column and condensed to produce a second distillate (207), the second set of organic impurities being produced as the second distillation column bottoms (208);
C) feeding the first distillate of step A (203) and the second distillate of step B (207) to a watering zone (209) and
C1) combining therein to produce a water enriched acetonitrile stream (210a), or
C2) combining therein and enriching with water to produce a water enriched acetonitrile stream (210); and
D) introducing the water enriched acetonitrile stream (210, 201a) into a third distillation column (212) and separating the first set of impurities from the acetonitrile, the acetonitrile being produced as the third distillation column bottoms (213);
such that recovered and/or purified acetonitrile is obtained.

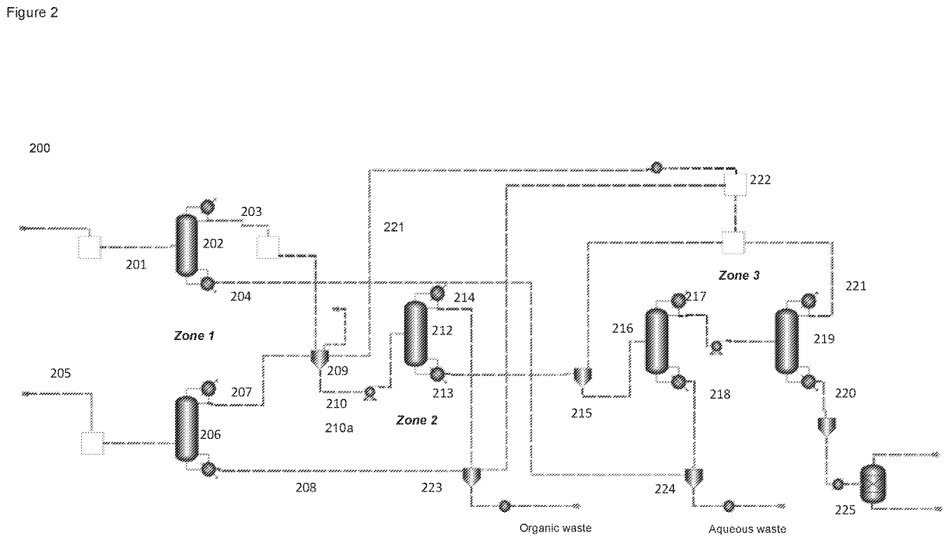

The distillations of steps A and B may be executed independently, e.g., one after the other in any order or ratio ranging from 0-100% or in parallel, e.g., concurrently. The steps may be started concurrently as the waste streams will be produced at the same rate from the main synthesis/purification sections of the process, although this is not essential. The process steps of A and B take place in a first distillation zone of the second aspect of the present disclosure (FIG. 2, zone 1).

In process steps A and B the inorganic aqueous and organic purification steps take place. The goal is that as many heavy boilers as possible are separated from acetonitrile and any other light boilers in the feed streams. The aqueous acetonitrile waste emanates from the purification area of the oligonucleotide process while the organic waste emanates from the oligonucleotide synthesis section. The bottoms product from each distillation is sent to waste. The top products are combined to produce an aqueous feed to the light boiler column (212) in the second distillation zone (FIG. 2, zone 2).

The aqueous acetonitrile waste (201) entering this zone is separated into a stream (203) enriched in acetonitrile and comprising water and ethanol. The heavier boiling portion (204) comprises non-volatile salts and organic residue from the main process purification as an aqueous solution. This zone is operated to maximize the yield of acetonitrile in the form of an azeotrope while removing as much excess water as possible from the process.

The organic waste stream (205) fed to this zone contains a mixture of acetonitrile, and organic impurities, such as 2,6-lutidine, toluene, acrylonitrile and lesser amounts of pyridine, imidazole, 1-methylimidazole, diethylamine, diisopropylamine, acetic acid, 5-ethylthio-1H-tetrazole, N,N-dimethylformamide, organic iodine and sulphur compounds, and residual oligonucleotide building blocks, as well as trace amounts of water. Many of these compounds are difficult to separate from acetonitrile as they either have very similar volatilities as acetonitrile within certain concentration ranges, or they form azeotropic mixtures with acetonitrile.

In step A, the acetonitrile fraction is drawn from the upper portion, e.g., from the top of the distillation column (202). The acetonitrile fraction leaves as a vapour and is passed through a condenser to produce a condensate (203). A portion of the condensate is then returned to the column (202) as a reflux stream and the remainder is removed as a distillate. The acetonitrile fraction (203) comprises acetonitrile and lighter boiling impurities, such as an acetonitrile/water azeotrope. The acetonitrile fraction (203) may additionally comprise ethanol. Heavy aqueous impurities, e.g., having a boiling temperature greater than acetonitrile, are drawn as a distillation column bottoms (204). The heavy boiling aqueous impurities comprise non-volatile salts and organic residue and are withdrawn as a bottoms product (204). The bottoms product (204) is considered as waste and is discarded. The bottoms product (204) or at least a portion thereof may be fed to a separate mixing zone (224), e.g., in the third distillation zone (FIG. 2, zone 3) whereby it is mixed with the aqueous bottoms product from the lower pressure distillation (218) in the third distillation zone, before being discarded.

The distillation of step B comprises introducing an organic waste feedstock (205) comprising acetonitrile and the aforementioned organic impurities into a distillation column (206), distilling said organic waste comprising acetonitrile to remove organic impurities therefrom and produce a first acetonitrile distillate (207) comprising acetonitrile and a first set of impurities comprising azeotrope formers and lower boiling impurities, and a second set of impurities comprising heavy organics, e.g., having a boiling temperature greater than acetonitrile. The acetonitrile distillate (207) along with the first set of impurities is withdrawn from the upper portion, e.g., from the top of the distillation column (206). The second set of impurities is produced as the distillation column bottoms product (208). The first set of impurities generally have a lower boiling temperature relative to water (100° C. under atmospheric pressure), in particular relative to acetonitrile (82° C. under atmospheric pressure). The azeotrope formers comprise at least one of toluene, ethanol, and acrylonitrile, or mixtures thereof. The low boiling impurities comprise at least one of diethylamine, diisopropylamine, and acrylonitrile, or mixtures thereof.

The second set of impurities comprising heavy organics (208) comprises at least one of toluene, acetic acid, and a N-containing compound, or mixtures thereof. In an embodiment, the N-containing compound is at least one of 1-methylimidazole, 2,6-lutidine, dimethylformamide, pyridine, acrylonitrile, diisopropylamine, diethylamine, phenyl acetyl(disulfide), and 5-(ethylthio)-1H-tetrazole,5-(ethylthio)tetrazole, or mixtures thereof.

The process steps C and D take place in the so-called second distillation zone (FIG. 2).

Step C comprises feeding the acetonitrile distillate of step A (203) and the acetonitrile distillate of step B (207) to a watering zone (209) and
combining therein, to produce a water enriched acetonitrile stream (210a), or
combining therein and enriching with water to produce a water enriched acetonitrile stream (210).

In an embodiment of step C, step C may comprise the additional step of feeding into the watering zone a recycled acetonitrile stream (221), wherein the recycled acetonitrile stream (221) is a by-product of a further processing step e.g., from a pressure swing distillation, e.g., steps F1 and F2. The recycled acetonitrile stream (221) comprises a composition at or approaching the acetonitrile/water azeotropic composition at the operating conditions of the column (219) of step F2. The stream (221) may further comprise ethanol, which builds-up in the system. The amount of ethanol present will vary depending on the feed concentrations and the flow rate of this stream relative to the feed stream (213) entering the pressure swing distillation steps, e.g., steps F1 and F2. The recycled acetonitrile stream (221) may comprise acetonitrile in a range of 70 to 76 wt. %, e.g., 75 wt. %, ethanol in a range of 0 to 3 wt. %, e.g., 1.3 wt. %, and water in a range of 24 to 30 wt. %, e.g., 24 wt. % based on the total content of the stream. Recycling 10 to 30% of this stream enables ethanol to be purged from the process in the form of an ethanol/water azeotrope. The recycling step ensures optimal removal of ethanol from the process.

The water enriched acetonitrile stream (210, 201a) is then fed into a distillation column (212). The water enriched acetonitrile feed (210, 210a) is distilled and separated into an acetonitrile bottoms product (213) and a lower boiling fraction (214) comprising lower boiling azeotropes. All of the impurities in the feed are separated from the upper portion, e.g., from the top of the column as binary or ternary azeotropes. The lower boiling azeotropes of fraction (214) are drawn from the upper portion, e.g., from the top of the distillation column (212). Such lower boiling azeotropic fraction (214) may be directed to organic waste. In an embodiment, at least a portion of said lower boiling azeotropic fraction (214) is first mixed, e.g., in a separate mixing zone (223), with other organic impurities, e.g., from a previous distillation step, e.g., the distillation column bottoms product (208) of step B, before being directed to organic waste.

Advantageously, the above process steps A to D provide an improved acetonitrile recovery and/or purification process in terms of efficiency. This process also assures that any light boiling substances that enter through aqueous waste stream (201) are purged from the process in the third distillation column (212) of step D in the form of a single component or in the form of an azeotrope. Such process can successfully remove high boiling hydrophobic organic and inorganic aqueous impurities from acetonitrile waste streams, e.g., those produced during oligonucleotide synthesis. The amount of acetonitrile that can be regenerated in the process employing steps A to D represents between 70 and 90% of the total acetonitrile consumed in the oligonucleotide synthesis process. The acetonitrile produced according to steps A to D is 99.90% (area %) pure according to gas chromatography analysis. Such recovered acetonitrile can be conveniently reused directly in any oligonucleotide synthesis processes.

In an embodiment of the present disclosure the distillation column (202) reflux ratio of step A is 0.25 to 2, e.g., 1. In an embodiment of the present disclosure the distillation column (206) reflux ratio of step B is 2 to 8, e.g., 5. In an embodiment of the present disclosure the distillation column (212) reflux ratio of step D is 5 to 40, e.g., 30.

The acetonitrile stream produced in step D may be subjected to further processing steps.

The acetonitrile enriched stream (e.g., 110, 216) may thereafter be fed to a de-watering zone (also referred to as the third distillation zone (FIG. 2), wherein the water content of the incoming acetonitrile stream (e.g., 110, 216) is reduced by use of a pressure-swing distillation to recover acetonitrile with a water content less than 30 ppm.

The acetonitrile recovered from the pressure-swing distillation step is more than 99.90% (area %) pure, e.g., when measured by gas chromatography. Preferably, the recovered acetonitrile has a purity of at least 99.90% (area %), e.g., when measured by gas chromatography and a water content of less than 30 ppm. Such recovered acetonitrile can be conveniently reused directly in any oligonucleotide synthesis processes, e.g., in solid-phase oligonucleotide synthesis processes.

In an embodiment, the process of the second aspect of present disclosure further comprises the steps:
F1) introducing the acetonitrile bottoms product (213) from the third distillation of step D into a fourth distillation column (216) and, performing a distillation at below atmospheric pressure to remove water therefrom, the acetonitrile being drawn as a vapour from said fourth distillation column and condensed to produce a fourth distillate (217), and water being produced as the fourth distillation column bottoms (218);
F2) introducing the fourth distillate (217) into a fifth distillation column (219) and, performing a second distillation at above atmospheric pressure, e.g., 5 bar, to produce lower boiling fraction (221) being drawn via the upper portion of the fifth distillation column (219) and acetonitrile being produced as the fifth distillation column bottoms (220).

The acetonitrile drawn from said column in step F2 is more than 99.90% (area %) pure when measured by GC based on the total content of the acetonitrile stream (220).

The acetonitrile fraction drawn via the upper portion, e.g., from the top of the distillation column in step F1 comprises an acetonitrile composition approaching the azeotropic composition. The lower boiling impurities of step F2 comprise at least one of ethanol, water, ethanol/water azeotrope and an acetonitrile/water azeotrope, or mixtures thereof.

Thereby it is ensured that the acetonitrile stream (217) reaching the fifth distillation column (219), in which the distillation of step F2 is performed, has a reduced water content and is as rich as possible in acetonitrile. During the distillation step F2, which is performed at a higher pressure than distillation step F1, the water content increases and reaches at least 23% by weight excluding the azeotropic composition, and produces a composition that approaches the azeotropic composition at the operating pressure of the column. Hence, the remaining water can be distilled away in the form of an acetonitrile/water azeotrope.

In an embodiment of the present disclosure the distillation column (216) reflux ratio of step F1 is 0.05 to 1, e.g., 0.1. In an embodiment of the present disclosure the distillation column (219) reflux ratio of step F2 is 0.05 to 1, e.g., 0.25.

The azeotropic acetonitrile composition (221) may be recycled to the acetonitrile feedstock for the distillation of step D, e.g., via the watering zone (209) of step C.

Thus, in an embodiment, step C may comprise the additional step of feeding into the watering zone a recycled acetonitrile stream (221), wherein the recycled acetonitrile stream (221) is a by-product of a further processing step, e.g., from the dewatering section (third distillation zone, FIG. 2). e.g., from a pressure swing distillation, e.g., steps F1 and F2, and comprises water, acetonitrile and ethanol, which builds-up in the system. The recycled acetonitrile stream (221) comprises an acetonitrile/water azeotrope and ethanol. Recycling 10 to 30% of this stream enables ethanol to be purged from the process in the form of the ethanol/water azeotrope. The recycling step ensures optimal removal of ethanol from the process.

In an embodiment, at least a portion of the acetonitrile/water azeotrope (221) is recycled back to step C with at least a proportion being sent back to a further mixing zone (215) (Step E).

In an embodiment, the process of the second aspect of present disclosure further comprises the step of mixing the acetonitrile bottoms product (213) from the third distillation of step D with a portion of a recycled azeotropic acetonitrile composition (221) from a further processing step, e.g., from a pressure swing distillation, e.g., step F2, before being subjected to steps F1 and F2 (Step E). The step of mixing the acetonitrile bottoms product (213) from the third distillation of step D with a recycled azeotropic acetonitrile composition (221) may take place in a further mixing zone (215). This embodiment may also include the prior step of feeding into the watering zone (209) a recycled acetonitrile stream (221) as disclosed above.

Thus, in an embodiment, any impurities present in the de-watering zone, e.g., in step F2, which may form light boiling azeotropes with water and acetonitrile are withdrawn from the upper portion, e.g., from the top of the column (221) and are recycled back to the second distillation zone, e.g., steps C, D and E, along with any remaining acetonitrile and water.

Alternatively, the lower boiling azeotropic fraction (221) from the de-watering zone, e.g., from step F2, may be purged (222) and sent to organic waste.

In the case that the water content in the acetonitrile product stream (220) from step F2 is not low enough, an additional stage of separation (225) can be added that could remove the residual water as an azeotrope or it could be removed by adsorption on to a stationary phase, e.g., molecular sieves.

Therefore, in a further embodiment of the disclosure, the process additionally comprises the step of feeding the acetonitrile bottoms product (220) from step F2 to a water adsorption zone (225) and contacting said acetonitrile bottoms product (220) with a water reducing adsorbent to produce acetonitrile with less than 30 ppm water that exits the adsorption zone (225). The water reducing adsorbent may be molecular sieves. Further details and embodiments for the adsorbent(s) are described at [0033] to [0038] of WO2015126713 A1. However, in a preferred embodiment, molecular sieves are used. In an embodiment, the molecular sieves have a pore size less than 20 Angstroms, e.g., 3, 4 or 5 Angstroms. In a preferred embodiment, the molecular sieves have a pore size of 3 Angstroms. Since water has a size less than this and acetonitrile has a size greater than this, a pore size of 3 Angstroms is very efficient for separation. The acetonitrile leaving the adsorption zone has purity of at least 99.90% (area %), e.g., when measured by gas chromatography and a water content of below 30 ppm.

In yet another aspect, there is provided a process for recovering and/or purifying acetonitrile from waste acetonitrile (301, 302) generated during oligonucleotide synthesis, the process comprising the steps:
A′) introducing an organic waste feedstock (301) comprising acetonitrile, a first set of organic impurities having a lower boiling temperature than acetonitrile and a second set of organic impurities having a boiling temperature greater than acetonitrile, and an aqueous waste feedstock (302) comprising acetonitrile a first set of aqueous impurities having a lower boiling temperature than acetonitrile and a second set of aqueous impurities having a boiling temperature greater than acetonitrile, into a first mixing zone (303), and combining therein, to form an acetonitrile waste feedstock comprising said impurities; or
introducing an organic waste feedstock (301) comprising acetonitrile, a first set of organic impurities having a lower boiling temperature than acetonitrile and a second set of organic impurities having a boiling temperature greater than acetonitrile, into a first mixing zone (303), and enriching with water, to produce a water enriched acetonitrile stream comprising said impurities;
B) introducing the acetonitrile waste feedstock or water enriched acetonitrile stream into a first distillation column (304) and separating the acetonitrile and the first set of impurities having a lower boiling temperature than acetonitrile from the second set of impurities having a boiling temperature greater than acetonitrile, the acetonitrile and first set of impurities being drawn as a vapour from said first distillation column and condensed to produce a first distillate (306), the second set of impurities being produced as the first distillation column bottoms (305);
C) optionally introducing the first distillate (306) comprising acetonitrile and low boiling impurities, into a watering zone (307) and enriching with water to produce a water enriched acetonitrile stream (307a); and
D′) introducing the water enriched acetonitrile stream (306, 307a) into a second distillation column (308) and separating the acetonitrile from the low boiling impurities, the acetonitrile being produced as the second distillation column bottoms (309);
such that recovered and/or purified acetonitrile is obtained.

The second distillation column bottoms (309) produced as a result of the second distillation step D′ comprises an acetonitrile/water azeotrope.

In order to reduce the water content of the acetonitrile product to less than 30 ppm, an additional step E) may be further employed by introducing the second distillation column bottoms product (309) of step D′ into a third distillation column (311) and separating the acetonitrile from the acetonitrile/water azeotrope, the acetonitrile being produced as the third distillation column bottoms (312).

Further details and embodiments of the fourth aspect of the present disclosure are disclosed Infra.

The following details and embodiments of the first, second, and third distillation zones are applicable to all aspects of the present disclosure, including the processes of the first, second, third and fourth aspects of the present disclosure, and may be combined.

### First Aspect of the Present Disclosure

In accordance with a first aspect, provided herein is a process for recovering and/or purifying acetonitrile from waste acetonitrile (105, 205) generated during oligonucleotide synthesis, the process comprising the steps:
A*) introducing an organic waste feedstock (105, 205) comprising acetonitrile, a first set of organic impurities having a lower boiling temperature than acetonitrile and a second set of organic impurities having a boiling temperature greater than acetonitrile, into a distillation column (106, 206) and separating the acetonitrile and first set of organic impurities from the second set of organic impurities, the acetonitrile and first set of organic impurities being drawn as a vapour from said distillation column and condensed to produce a distillate (107, 207), the second set of organic impurities being produced as the second distillation column bottoms (108, 208);
B*) introducing the distillate (107, 207) into a watering zone (109, 209) to produce a water enriched acetonitrile stream (111, 210, 210a);
C*) introducing the water enriched acetonitrile stream (111, 210, 210a) into a second distillation column (112, 212) and separating the first set of organic impurities from the acetonitrile, the acetonitrile being produced as the second distillation column bottoms (113, 213);
such that recovered and/or purified acetonitrile is obtained.

The acetonitrile produced as the second distillation column bottoms can be collected and reused or can be further processed by employing steps A1 and E1 or steps A and C according to the second or third aspect of the present disclosure. By further employing these steps, the yield of the recovered acetonitrile can be improved. Thus, the separation of acetonitrile from aqueous waste process streams is not essential to produce recovered and/or purified acetonitrile from organic waste streams in the context of the present disclosure.

The light boiling acetonitrile stream (107, 207), which is fed to a watering zone (109, 209), comprises light boiling impurities (107, 207) and is enriched with water to produce a water enriched acetonitrile stream (111, 210, 210a). Several compounds in the feed to the watering zone are rendered lighter boiling in the form of either binary or ternary azeotropes, for example toluene/water/acetonitrile, acetonitrile/water/diisopropylamine, acetonitrile/water/ethanol, acetonitrile/water/ethyl acetate.

The water can be directly added to the first acetonitrile distillate (107, 207) or may be added to the first acetonitrile distillate (107, 207) as a component of a further acetonitrile process stream, e.g., present in an acetonitrile distillate from an aqueous purification step. The addition of water via an acetonitrile process stream advantageously avoids the need for the additional enrichment with water.

By incorporating a watering zone, separation of otherwise complex mixtures of light boiling impurities and azeotrope formers, such as toluene, acrylonitrile, ethanol, ethyl acetate, and low boiling amines, among others, can be achieved. The acetonitrile produced according to the steps A* to C* has a purity of at least 99.90%. All of the impurities in the feed (111, 210, 210a) are separated from the upper portion, e.g., from the top of the column (112, 212) as binary or ternary azeotropes with the acetonitrile being produced as the second distillation column bottoms (113, 213). Thus, by utilizing water as an entrainer, successful partitioning and grouping of organic waste from acetonitrile process streams can be achieved.

Further details and embodiments of the first aspect of the present disclosure for the distillation of step A* are equivalent to those disclosed according to step B1 or step B according to the second or third aspect of the present disclosure and are provided Infra.

Further details and embodiments of the first aspect of the present disclosure for the distillation of step C* are equivalent to those disclosed according to step D1 or step D according to the second or third aspect of the present disclosure and are provided Infra.

### Second Aspect of the Present Disclosure

### First Distillation Zone

The first distillation zone can be termed the organic purification zone and comprises at least two distillation columns (106, 112). The organic waste stream fed to this zone contains a combination of acetonitrile, 2,6-lutidine, acetic anhydride, dichloroacetic acid, pyridine, toluene, 1-methyl imidazole, and lesser amounts of diethylamine, diisopropylamine, acetic acid, 5-ethylthio-1H-tetrazole, N,N-dimethylformamide, organic iodine and sulphur compounds, and residual oligonucleotide building blocks and trace amounts of water. The heavy organics produced as a bottoms product (108) in step B1 are considered waste. The concentration of compounds in solution can be present at concentrations ranging from fractions of up to 10 wt %. Many of these compounds are difficult to separate from acetonitrile as they either have very similar volatilities as acetonitrile within certain concentration ranges, or they form azeotropic mixtures with acetonitrile. A number of purification stages in this zone can be employed to separate acetonitrile from the other compounds present. One stage can be used to first separate a heavy boiling fraction of compounds from acetonitrile, light boilers and azeotrope formers. This stage can be designed to take advantage of even the smallest difference in relative volatility of the compounds to be separated to increase the purity of the acetonitrile stream. The light boiling fraction (107) from this stage containing the majority of the acetonitrile can then be fractionated in a further stage, e.g., in a second distillation column (112), to produce a substantially pure acetonitrile product stream (113) and a more volatile light boiler waste stream (114). In order to separate azeotrope formers with acetonitrile, such as toluene, acrylonitrile or ethanol, an entrainer such as water can be added (109) to the feed that will produce lower boiling azeotropes with these compounds relative to acetonitrile making them easier to separate from acetonitrile (111).

The organic waste stream (105) of the present disclosure which enters the first distillation zone comprises acetonitrle. In some embodiments, the organic waste stream (105) comprises a relatively high content of acetonitrile. In one embodiment, the organic waste stream (105) comprises acetonitrle in an amount ranging from 70 wt. % to 95 wt. % acetonitrile, based on the total weight of the feedstock stream (105). e.g., from 75 wt. % to 95 wt. %, from 80 wt. % to 94 wt. %, from 85 wt. % to 93 wt. %, or from 87 wt. % to 92 wt. % acetonitrile, e.g., 90 wt. %. In terms of upper limits, the feedstock stream may comprise less than 95 wt. % acetonitrile, e.g., less than 94 wt. %, less than 93 wt. %, less than 92 wt. %, or less than 91 wt. % acetonitrile. In terms of lower limits, the feedstock stream may comprise greater than 70 wt. % acetonitrile, e.g., greater than 75 wt. %, greater than 80 wt. %, greater than 85 wt. %, greater than 86 wt. %, greater than 87 wt. %, greater than 88 wt. %, or greater than 89 wt. %, acetonitrile.

In some embodiments, the distillation column (106) of step B1 operates in a range of 50 to 980 mbar, preferably at 100 to 300 mbar. In terms of lower limits, distillation column (106) may operate at a pressure greater than 50 mbar, e.g., greater than 100, greater than 200, greater than 300 mbar, greater than 400 mbar, greater than 500 mbar, greater than 600 mbar, greater than 700 mbar, or greater than 800 mbar. In terms of upper limits, the first distillation column may operate a pressure less than 900 mbar, e.g., less than 800 mbar, less than 700 mbar, less than 600 mbar, less than 500 mbar, less than 400 mbar, less than 300 mbar, or less than 200 mbar.

Generally, distillation column (106) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (106) reflux ratio of step B1 is 2 to 8, e.g., 5.

The light boiling fraction (107) produced in step B1 comprises about 90 wt. % to 99.5 wt. % acetonitrile. In some embodiments, the light boiling fraction (107) comprises acetonitrile in an amount ranging from 96 wt. % to 99.5 wt. % acetonitrile, based on the total weight of the distillate (107), e.g., from 96.5 wt. % to 99.5 wt. %, from 97 wt. % to 99.5 wt. %, from 97.5 wt. % to 99.5 wt. %, or from 98 wt. % to 99.5 wt. % acetonitrile. In terms of upper limits, the light boiling fraction (107) may comprise less than 99.5 wt. % acetonitrile, e.g., less than 99 wt. %, less than 98.5 wt. %, less than 98 wt. %, less than 97.5 wt. %, less than 97 wt. %, less than 96.5 wt. %, less than 96 wt. %, less than 95.5 wt. %, or less than 95 wt. % acetonitrile. In terms of lower limits, the light boiling fraction (107) may comprise greater than 90 wt. % acetonitrile, e.g., greater than 91 wt. %, greater than 92 wt. %, greater than 93 wt %, greater than 94 wt %, greater than 95 wt %, greater than 95.5 wt %, greater than 96 wt %, greater than 96.5 wt %, greater than 97 wt %, greater than 97.5 wt %, greater than 98 wt %, greater than 98.5 wt %, or greater than 99 wt % acetonitrile.

Those impurities having a boiling point between the acetonitrile azeotrope boiling points and the acetonitrile boiling point, will be withdrawn together with the low boiling impurities, acetonitrile azeotropes and the acetonitrile. The light boiling fraction is drawn from the upper portion from the column, e.g., from the top of the column. The azeotrope formers comprise toluene, ethanol, water and acrylonitrile. This fraction still contains high boiling hydrophobic impurities, such as toluene. This happens, because such high boiling hydrophobic impurities can form hydrophobic-hydrophobic interactions with acetonitrile and thereby are dragged over the column top during distillation. Hence, the fraction withdrawn from the top of the column comprises acetonitrile as well as light boiling impurities, such as binary and ternary azeotropes, such as acetonitrile/diethylamine, acetonitrile/diisopropylamine, toluene/water/acrylonitrile, toluene/water/acetonitrile, diisopropylamine/water/acetonitrile, acetonitrile/water, acetonitrile/toluene, toluene/water, or ethanol/water, or mixtures thereof. Light boiling impurities may additionally comprise at least one N-containing compound, such as diethylamine, diisopropylamine, and acrylonitrile, or mixtures thereof.

The light boiling fraction (107) produced in step B1 may also comprise ethanol as an impurity. In some embodiments, the light boiling fraction (107) comprises a relatively low content of ethanol. In one embodiment, the light boiling fraction (107) comprises ethanol in an amount ranging from 0.5 wt. % to 3 wt. % ethanol, based on the total weight of the fraction (107), e.g., from 0.6 wt. % to 3 wt. %, from 0.7 wt. % to 3 wt. %, from 0.8 wt. % to 3 wt. % ethanol. In terms of upper limits, the light boiling fraction (107) may comprise less than 3 wt. % ethanol, e.g., less than 2.8 wt. %, less than 2.6 wt. %, less than 2.4 wt. %, less than 2.2 wt. %, less than 2 wt. %, less than 1.5 wt. % ethanol. In terms of lower limits, the light boiling fraction (107) may comprise greater than 0.5 wt. % ethanol, e.g., greater than 0.6 wt. %, greater than 0.7 wt. %, greater than 0.8 wt. %, greater than 0.9 wt. %, greater than 1 wt. %, greater than 1.2 wt. %, greater than 1.5 wt. %, greater than 1.7 wt. %, greater than 2 wt. %, or greater than 2.5 wt. % ethanol.

In some embodiments, the light boiling fraction (107) comprises toluene in an amount ranging from 0.2 wt. % to 5 wt. % toluene, e.g., from 0.5 wt. % to 5 wt. %, from 1 wt. % to 5 wt. %, from 1.5 wt. % to 5 wt. %, from 2 wt. % to 5 wt. %, from 2.5 wt. % to 5 wt. %, from 3 wt. % to 5 wt. %, from 3.5 wt. % to 5 wt. % toluene. In terms of upper limits, the light boiling fraction (107) may comprise less than 5 wt. % toluene, e.g., less than 4.5 wt. %, less than 4 wt. %, less than 3.5 wt. % toluene, less than 3 wt. %, less than 2.5 wt. %, less than 2 wt. %, less than 1.5 wt. %, less than 1 wt. %, or less than 0.5 wt. % toluene. In terms of lower limits, the light boiling fraction (107) may comprise greater than 0.2 wt. % toluene, e.g., greater than 0.5 wt. %, greater than 0.8 wt. %, greater than 1 wt. %, greater than 1.2 wt. %, greater than 1.5 wt. %, greater than 1.8 wt. %, greater than 2 wt. %, greater than 2.2 wt. %, greater than 2.5 wt. %, greater than 2.8 wt. %, greater than 3 wt. %, greater than 3.2 wt. %, greater than 3.5 wt. %, greater than 3.8 wt. %, greater than 4 wt. %, or greater than 4.5 wt. % toluene.

In some embodiments, the light boiling fraction (107) comprises acrylonitrile in an amount ranging from 1 to 200 ppm, e.g., from 5 to 180 ppm, from 50 to 150 ppm acrylonitrile. In terms of upper limits, the light boiling fraction (107) may comprise less than 200 ppm acrylonitrile, e.g., less than 180 ppm, less than 150 ppm, less than 120 ppm, less than 100 ppm, less than 75 ppm, less than 50 ppm, or less than 20 ppm acrylonitrile. In terms of lower limits, the light boiling fraction (107) may comprise greater than 1 ppm acrylonitrile, e.g., greater than 5 ppm, greater than 10 ppm, greater than 20 ppm, greater than 30 ppm, greater than 40 ppm, greater than 50 ppm, greater than 60 ppm, greater than 70 ppm, greater than 80 ppm, greater than 90 ppm, greater than 100 ppm, greater than 120 ppm, or greater than 150 ppm acrylonitrile.

In some embodiments, the light boiling fraction (107) comprises diethylamine in an amount ranging from 5 to 300 ppm, e.g., from 10 to 300 ppm, from 20 to 280 ppm from 50 to 250 ppm diethylamine. In terms of upper limits, the light boiling fraction (107) may comprise less than 300 ppm diethylamine, e.g., less than 280 ppm, less than 250 ppm, less than 230 ppm, less than 210 ppm, less than 200 ppm, less than 180 ppm, less than 150 ppm, less than 130 ppm, less than 100 ppm, or less than 50 ppm diethylamine. In terms of lower limits, the light boiling fraction (107) may comprise greater than 5 ppm diethylamine e.g., greater than 20 ppm, greater than 50 ppm, greater than 75 ppm, greater than 100 ppm, greater than 120 ppm, greater than 150 ppm, greater than 175 ppm, greater than 200 ppm, greater than 250 ppm diethylamine.

In some embodiments, the light boiling fraction (107) comprises diisopropylamine in an amount ranging from 10 to 600 ppm, e.g., from 50 to 550 ppm, from 75 to 500 ppm, from 100 to 400 ppm diisopropylamine. In terms of upper limits, the light boiling fraction (107) may comprise less than 600 ppm diisopropylamine, e.g., less than 550 ppm, less than 500 ppm, less than 450 ppm, less than 400 ppm, less than 350 ppm, less than 300 ppm, less than 250 ppm, less than 200 ppm, or less than 150 ppm diisopropylamine. In terms of lower limits, the light boiling fraction (107) may comprise greater than 10 ppm diisopropylamine, e.g., greater than 50 ppm, greater than 75 ppm, greater than 100 ppm, greater than 150 ppm, greater than 175 ppm, greater than 200 ppm, greater than 250 ppm, greater than 300 ppm greater than 350 ppm greater than 400 ppm greater than 550 ppm, or greater than 500 ppm diisopropylamine.

The light boiling fraction (107) leaves as a vapour and is passed through a condenser to produce a condensate. A portion of the condensate is then returned to the column as a reflux stream and the remainder is removed as a product.

The heavier boiling organic impurities that are drawn as a bottoms product (108) from the distillation column (106) are considered as waste and are discarded. In some embodiments, the heavier boiling organic impurities comprise at least one of 1-methylimidazole, 2,6-lutidine, acetic acid, dimethylformamide, ETT, PADS, pyridine and toluene, or mixtures thereof.

In order to purify the acetonitrile from light boiling organic impurities present in the light boiling fraction (107) from the distillation of step B1, the resultant acetonitrile stream (107) is then fed to a watering zone (109), where water can be added to the feed that will produce lower boiling azeotropes with these impurity compounds relative to acetonitrile making them easier to separate from acetonitrile (111). The amount of water added may be anywhere between 0.05 to 0.40 w/w based on the amount of acetonitrile present in the stream. Preferably, the lower boiling azeotropes present in the acetonitrile stream (111) comprise at least one of acetonitrile/water, acrylonitrile/water, acetonitrile/toluene, acetonitrile/diethylamine, acetonitrile/diisopropylamine, toluene/water, ethanol/water, toluene/water/acrylonitrile, toluene/water/acetonitrile, diisopropylamine/water/acetonitrle, and diethylamine/toluene/water, or mixtures thereof.

The water enriched acetonitrile stream (111) is then fed to distillation column (112) and comprises about 5 wt. % to 40 wt. % acetonitrile based on the total weight of the water enriched acetonitrile stream (111). In some embodiments, the water enriched acetonitrile stream (111) comprises acetonitrile in an amount ranging from 10 wt. % to 40 wt. %, e.g., from 15 wt. % to 35 wt. % acetonitrile. In terms of upper limits, the water enriched acetonitrile stream (111) may comprise less than 40 wt. % acetonitrile, e.g., less than 35 wt. %, less than 30 wt. %, or less than 20 wt. % acetonitrile. In terms of lower limits, the water enriched acetonitrile stream (111) may comprise greater than 5 wt. % acetonitrile, e.g., greater than 10 wt. %, greater than 20 wt. %, or greater than 30 wt. %, acetonitrile.

In some embodiments, the distillation of step D1 operates in a range of 100-980 mbar, preferably at 900 to 980 mbar. In terms of lower limits, distillation column (112) may operate at a pressure greater than 100 bar, e.g., greater than 200 mbar, greater than 300 mbar, greater than 400 mbar, greater than 500 mbar, greater than 600 mbar, greater than 700 mbar, or greater than 800 mbar. In terms of upper limits, distillation column (112) may operate a pressure less than 980 mbar, e.g., less than 950 mbar, less than 900 mbar, or less than 800 mbar.

Generally, distillation column (112) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (112) reflux ratio of step D1 is 5 to 40, e.g., 30.

In step D1, the water enriched acetonitrile stream (111) is introduced into distillation column (112) and separated into a lower boiling azeotropic fraction (114) and an acetonitrile distillation column bottoms product (113). The lower boiling azeotropic fraction (114) comprises at least one of acetonitrile/water, acrylonitrile/water, acetonitrile/toluene, acetonitrile/diethylamine, acetonitrile/diisopropylamine, toluene/water, ethanol/water, toluene/water/acrylonitrile, toluene/water/acetonitrile, diisopropylamine/water/acetonitrile, and diethylamine/toluene/water, or mixtures thereof.

The lower boiling azeotropic fraction (114) leaves as a vapour and is passed through a condenser to produce a condensate. The lower boiling azeotropic fraction (114) is drawn as a vapour from the upper portion, e.g., from the top the column (112).

The acetonitrile drawn from distillation column (112) as a bottoms product (113) comprises about 60 to 90 wt. % acetonitrile. In one embodiment, the acetonitrile bottoms product (113) comprises acetonitrile in an amount ranging from 65 wt. % to 90 wt. % acetonitrile, based on the total weight of the recovered acetonitrile bottoms product (113), e.g., from 65 wt. % to 85 wt. % acetonitrile. In terms of upper limits, the acetonitrile bottoms product (113) may comprise less than 90 wt. % acetonitrile, e.g., less than 85 wt. %, less than 80 wt. %, less than 75 wt. %, or less than 70 wt. % acetonitrile. In terms of lower limits, the acetonitrile bottoms product (113) may comprise greater than 65 wt. % acetonitrile, e.g., greater than 70 wt. %, greater than 75 wt. %, greater than 80 wt. %, or greater than 85 wt. % acetonitrile.

The lower boiling acetonitrile azeotropes (114) that are withdrawn from the upper portion, e.g., from the top of distillation column (112), are considered as waste and are discarded.

Thereby, it is ensured that the acetonitrile stream (113) reaching the distillation column (117, 120) in the third distillation zone has a reduced water and impurity content and is as rich as possible in acetonitrile. In an embodiment, the acetonitrile recovered (113) according to steps A1 to D1 in accordance with the first aspect of the disclosure is at least 99.90% (area %) pure when measured by gas chromatography (GC) analysis.

The acetonitrile stream produced in step D1 may be subjected to further processing steps. Thus, in an embodiment the acetonitrile bottoms product (113) from step D1 and the acetonitrile distillate (103) from step A1 may thereafter be fed to a mixing zone (116) and combined therein, to an acetonitrile enriched stream (110) (step E1).

The acetonitrile enriched stream (110) comprises about 60 to 90 wt. % acetonitrile. In one embodiment, the acetonitrile enriched stream (110) comprises acetonitrile in an amount ranging from 65 wt. % to 90 wt. % acetonitrile, based on the total weight of the respective stream, e.g., from 65 wt. % to 85 wt. % acetonitrile. In terms of upper limits, the stream (110) may comprise less than 90 wt. % acetonitrile, e.g., less than 85 wt. %, less than 80 wt. %, less than 75 wt. %, or less than 70 wt. % acetonitrile. In terms of lower limits, the stream (110) may comprise greater than 65 wt. % acetonitrile, e.g., greater than 70 wt. %, greater than 75 wt. %, greater than 80 wt. %, or greater than 85 wt. % acetonitrile.

In one embodiment, the acetonitrile enriched stream (110) comprises up to 1 wt. % ethanol. In a further embodiment, the acetonitrile enriched stream (110) comprises up to 20 ppm pyridine. In a further embodiment, the acetonitrile enriched stream (110) comprises up to 50 ppm toluene.

### Second Distillation Zone

The second distillation zone can be termed an inorganic aqueous purification zone. The acetonitrile waste (101) entering this zone is separated into a light boiling stream (103) enriched in acetonitrile and containing some water and ethanol. The heavy boiling portion (104) contains non-volatile salts and organic residue from the main process purification as an aqueous solution. This zone is operated to maximize the yield of acetonitrile in the form of an azeotrope while removing as much excess water as possible from the process.

The aqueous waste stream (101) entering the inorganic purification zone (herein referred to as the second distillation zone) of the first aspect of the present disclosure comprises acetonitrile. In some embodiments, the aqueous waste stream (101) comprises a relatively low content of acetonitrile. In one embodiment, the aqueous waste stream (101) comprises acetonitrile in an amount ranging from 5 wt. % to 13 wt. %, based on the total weight of the aqueous waste stream (101), e.g., from 6 wt. % to 13 wt. %, e.g., 9.5 wt. %. In terms of upper limits, the aqueous waste stream (101) may comprise less than 15 wt. % acetonitrile, e.g., less than 14 wt. %, less than 13 wt. %, less than 12 wt. %, less than 11 wt. %, or less than 10 wt. % acetonitrile. In terms of lower limits, the aqueous waste stream (101) may comprise greater than 5 wt. % acetonitrile, e.g., greater than 6 wt. % acetonitrile, greater than 7 wt. % acetonitrile, greater than 8 wt. %, or greater than 9 wt. % acetonitrile.

Generally, as used herein, the weight percentages are based on the total weight of the respective stream. With respect to the aqueous waste stream (101), the weight percentages include all components of the feedstock, including a significant portion of water. In some embodiments, for example, the aqueous waste stream (101) comprises at least 87 wt. % water, e.g., at least 88 wt. %, at least 89 wt. %, or at least 90 wt. % water.

The aqueous waste stream (101) of the present disclosure additionally comprises ethanol. In some embodiments, the aqueous waste stream (101) comprises relatively low amounts of ethanol. In one embodiment, the aqueous waste stream (101) comprises ethanol in an amount ranging from 0.05 wt. % to 0.8 wt. %, based on the total weight of the aqueous waste stream (101), e.g., from 0.08 wt. % to 0.6 wt. %, from 0.1 wt. % to 0.5 wt. %, e.g., 0.2 wt. %. In terms of upper limits, the aqueous waste stream (101) may comprise less than 0.8 wt. % ethanol, e.g., less than 0.7 wt. %, less than 0.6 wt. %, or less than 0.5 wt. % ethanol. In terms of lower limits, the aqueous waste stream (101) may comprise greater than 0.08 wt. % ethanol, e.g., greater than 0.1 wt. %, greater than 0.15 wt. %, greater than 0.18 wt. %, greater than 0.2 wt. %, or greater than 0.5 wt. % ethanol.

The aqueous waste stream (101) is fed into a distillation column (102), whereby the light boiling fraction (103) comprising acetonitrile, water and ethanol is withdrawn from the upper portion, e.g., from the top of the column (102). The light boiling fraction (103) leaves as a vapour and is passed through a condenser to produce a condensate.

The light boiling acetonitrile fraction (103) drawn from the top of column (102) comprises about 70 wt. % acetonitrile. In some embodiments, the light boiling fraction (103) comprises acetonitrile in an amount ranging from 60 wt. % to 80 wt. % acetonitrile, based on the total weight of the recovered light boiling fraction (103). In terms of upper limits, the light boiling fraction (103) may comprise less than 80 wt. % acetonitrile, e.g., less than 78 wt. %, less than 75 wt. %, less than 73 wt. %, or less than 70 wt. % acetonitrile. In terms of lower limits, the light boiling fraction (103) may comprise greater than 60 wt. % acetonitrile, e.g., greater than 65 wt. %, greater than 68 wt. %, or greater than 70 wt. % acetonitrile.

In some embodiments, the light boiling fraction (103) comprises water in an amount ranging from 20 wt. % to 40 wt. % water, based on the total weight of the recovered light boiling fraction (103), e.g., from 22 wt. % to 38 wt. % water. In terms of upper limits, the light boiling fraction (103) may comprise less than 40 wt. % water, e.g., less than 38 wt. %, less than 35 wt. %, less than 33 wt. %, or less than 30 wt. % water. In terms of lower limits, the light boiling fraction (103) may comprise greater than 20 wt. % water, e.g., greater than 22 wt. %, greater than 25 wt. %, greater than 28 wt. %, greater than 30 wt. %, or greater than 35 wt. % water. The wt. % water present includes water present in azeotropic and non-azeotropic form.

In some embodiments, the light boiling fraction (103) comprises ethanol in an amount ranging from 0 wt. % to 2 wt. % ethanol, based on the total weight of the recovered light boiling fraction (103), e.g., from 0.05 wt. % to 2 wt. %, from 0.1 wt. % to 2 wt. %, from 0.5 wt. % to 2 wt. % ethanol. In terms of upper limits, the light boiling fraction (103) may comprise less than 2 wt. % ethanol, e.g., less than 1.8 wt. %, less than 1.5 wt. %, or less than 1 wt. % ethanol. In terms of lower limits, the light boiling fraction (103) may comprise greater than 0.05 wt. % ethanol, e.g., greater than 0.1 wt. %, greater than 0.5 wt. %, greater than 1 wt. %, greater than 1.5 wt. % greater than 1.75 wt. % ethanol.

In some embodiments, the distillation of step A1 operates at in a range of 200-980 mbar, preferably at 400 to 500 mbar. In terms of lower limits, the distillation column (102) may operate at a pressure greater than 200 mbar, e.g., greater than 300 mbar, greater than 400 mbar, greater than 500 mbar, greater than 600 mbar, greater than 700 mbar, or greater than 800 mbar. In terms of upper limits, the distillation (102) column may operate a pressure less than 980 mbar, e.g., less than 900, less than 800, less than 700 bar, less than 600 mbar, or less than 500 mbar.

Generally, distillation column (102) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required.

In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (102) reflux ratio of step A1 is 0.25 to 2, e.g., 1.

The light boiling fraction (103) is then fed to the mixing zone (116), where it is mixed with the substantially pure acetonitrile bottoms product (113) from step D1 (step E1). The resultant acetonitrile enriched stream (110) comprises about 76 wt. % acetonitrile. In some embodiments, the mixture (110) comprises acetonitrile in an amount ranging from 60 wt. % to 85 wt. % acetonitrile, based on the total weight of the acetonitrile enriched stream (110). In terms of upper limits, the acetonitrile enriched stream (110) may comprise less than 85 wt. % acetonitrile, e.g., less than 80 wt. %, less than 75 wt. %, or less than 70 wt. % acetonitrile. In terms of lower limits, the acetonitrile enriched stream (110) may comprise greater than 60 wt. % acetonitrile, e.g., greater than 65 wt. %, greater than 70 wt. %, greater than 75 wt. %, or greater than 80 wt. % acetonitrile.

The higher boiling aqueous bottoms product (104) of step A1 comprising non-volatile salts and organic residue is withdrawn as a bottoms product. The bottoms product (104) is considered as waste and is discarded. The aqueous bottoms product (104) may be fed to a second mixing zone (125), e.g., in the third distillation zone whereby it is mixed with further aqueous waste (119), e.g., from the de-watering steps, e.g., pressure swing distillation, e.g., steps F1 and F2, before being discarded.

### Third Aspect of the Present Disclosure

### First Distillation Zone

The first distillation zone of the third aspect of the present disclosure can be termed the dual aqueous and organic purification zone and comprises at least two distillation columns (202, 206). The process steps A and B occur in the so-called first distillation zone (FIG. 2), in which the organic and inorganic aqueous purification steps take place. The goal is that as many heavy boilers as possible are separated from acetonitrile and any other light boilers in the feed streams. The aqueous acetonitrile waste (201) emanates from the purification area of the oligonucleotide process while the organic waste (205) emanates from the oligonucleotide synthesis section. The bottoms product (204, 208) from each section is sent to waste. The top products (203, 207) are combined and enriched with water to produce an aqueous feed (210) to the light boiler column (212) in the second distillation zone (FIG. 2).

The aqueous acetonitrile waste (201) entering this zone is separated into a light boiling stream (203) enriched in acetonitrile and containing some water and ethanol. The heavy boiling portion (204) contains non-volatile salts and organic residue from the main process purification as an aqueous solution. This zone is operated to maximize the yield of acetonitrile in the form of an azeotrope while removing as much excess water as possible from the process.

The aqueous acetonitrile waste stream (201) entering the first distillation zone of the second aspect of the present disclosure comprises acetonitrile. In some embodiments, the aqueous waste stream (201) comprises a relatively low content of acetonitrile. In one embodiment, the aqueous waste stream (201) comprises acetonitrile in an amount ranging from 5 wt. % to 13 wt. %, based on the total weight of the feedstock stream (201), e.g., from 6 wt. % to 13 wt. %, e.g., 9.5 wt. %. In terms of upper limits, the aqueous waste feedstock stream (201) may comprise less than 15 wt. % acetonitrile, e.g., less than 14 wt. %, less than 13 wt. %, less than 12 wt. %, less than 11 wt. %, or less than 10 wt. % acetonitrile. In terms of lower limits, the aqueous waste feedstock stream (201) may comprise greater than 5 wt. % acetonitrile, e.g., greater than 6 wt. % acetonitrile, greater than 7 wt. % acetonitrile, greater than 7 wt. %, or greater than 9 wt. % acetonitrile.

Generally, as used herein, the weight percentages are based on the total weight of the respective stream. With respect to the aqueous waste stream (201), the weight percentages include all components of the feedstock, including a significant portion of water. In some embodiments, for example, the aqueous waste stream (201) comprises at least 87 wt. % water, e.g., at least 88 wt. %, at least 89 wt. %, or at least 90 wt. % water.

The aqueous waste stream (201) of the present disclosure additionally comprises ethanol. In some embodiments, the aqueous waste stream (201) comprises relatively low amounts of ethanol. In one embodiment, the aqueous waste stream (201) comprises ethanol in an amount ranging from 0.05 wt. % to 0.8 wt. %, based on the total weight of the feedstock stream (201), e.g., from 0.08 wt. % to 0.6 wt. %, from 0.1 wt. % to 0.5 wt. %, e.g., 0.2 wt. %. In terms of upper limits, the aqueous waste feedstock stream (201) may comprise less than 0.8 wt. % ethanol, e.g., less than 0.7 wt. %, less than 0.6 wt. %, or less than 0.5 wt. % ethanol. In terms of lower limits, the feedstock stream may comprise greater than 0.08 wt. % ethanol, e.g., greater than 0.1 wt. %, greater than 0.15 wt. %, greater than 0.18 wt. %, or greater than 0.2 wt. % ethanol.

The aqueous waste stream (201) is fed into a distillation column (202), whereby the light boiling fraction (203) comprising acetonitrile, water and ethanol is withdrawn from the upper portion, e.g., from the top of the column (202). The light boiling fraction (203) leaves as a vapour and is passed through a condenser to produce a condensate.

The light boiling acetonitrile fraction (203) drawn from the top of column (202) comprises about 70 wt. % acetonitrile. In some embodiments, the light boiling acetonitrile fraction (203) comprises acetonitrile in an amount ranging from 60 wt. % to 80 wt. % acetonitrile, based on the total weight of the recovered light boiling fraction (203). In terms of upper limits, the light boiling acetonitrile fraction (203) may comprise less than 80 wt. % acetonitrile, e.g., less than 78 wt. %, less than 75 wt. %, less than 73 wt. %, or less than 70 wt. % acetonitrile. In terms of lower limits, the light boiling acetonitrile fraction (203) may comprise greater than 60 wt. % acetonitrile, e.g., greater than 65 wt. %, greater than 68 wt. %, or greater than 70 wt. % acetonitrile.

In some embodiments, the light boiling acetonitrile fraction (203) comprises water in an amount ranging from 20 wt. % to 40 wt. % water, based on the total weight of the recovered light boiling fraction (203), e.g., from 22 wt. % to 38 wt. % water. In terms of upper limits, the feedstock stream may comprise less than 40 wt. % water, e.g., less than 38 wt. %, less than 35 wt. %, less than 33 wt. %, or less than 30 wt. % water. In terms of lower limits, the feedstock stream may comprise greater than 20 wt. % water, e.g., greater than 22 wt. %, greater than 25 wt. %, greater than 28 wt. %, greater than 30 wt. %, or greater than 35 wt. % water. The wt. % water present includes water present in azeotropic and non-azeotropic form.

In some embodiments, the light boiling acetonitrile fraction (203) comprises ethanol in an amount ranging from 0 wt. % to 2 wt. % ethanol, based on the total weight of the recovered light boiling fraction (203), e.g., from 0.05 wt. % to 2 wt. %, from 0.1 wt. % to 2 wt. %, from 0.5 wt. % to 2 wt. % ethanol. In terms of upper limits, the light boiling fraction (203) may comprise less than 2 wt. % ethanol, e.g., less than 1.8 wt. %, less than 1.5 wt. %, or less than 1 wt. % ethanol. In terms of lower limits, the light boiling fraction (203) may comprise greater than 0.05 wt. % ethanol, e.g., greater than 0.1 wt. %, greater than 0.5 wt. %, greater than 1 wt. %, greater than 1.5 wt. % greater than 1.75 wt. % ethanol.

In some embodiments, the distillation of step A operates at in a range of 200-980 mbar, preferably at 400 to 500 mbar. In terms of lower limits, the distillation column (201) may operate at a pressure greater than 200 mbar, e.g., greater than 300 mbar, greater than 400 mbar, greater than 500 mbar, greater than 600 mbar, greater than 700 mbar, or greater than 800 mbar. In terms of upper limits, the distillation column (201) may operate a pressure less than 980 mbar, e.g., less than 900, less than 800, less than 700 bar, less than 600 mbar, or less than 500 mbar.

Generally, distillation column (202) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (202) reflux ratio of step A is 0.25 to 2, e.g., 1.

The higher boiling aqueous mixture comprising non-volatile salts and organic residue is withdrawn as a bottoms product (204). The bottoms product (204) is considered as waste and is discarded. The aqueous bottoms product (204) may be fed to a second mixing zone (224), e.g., in the third distillation zone whereby it is mixed with other aqueous waste from the third distillation zone, e.g., the aqueous bottoms product (218), before being discarded.

The organic waste stream (205) fed to this zone contains a combination of acetonitrile, 2,6-lutidine, acetic anhydride, dichloroacetic acid, pyridine, toluene, 1-methyl imidazole, and lesser amounts of diethylamine, diisopropylamine, acetic acid, 5-ethylthio-1H-tetrazole, N,N-dimethylformamide, organic iodine and sulphur compounds, and residual oligonucleotide building blocks and trace amounts of water. The heavy organics produced as a bottoms product (208) in the first distillation zone of step B are considered waste. The concentration of compounds in solution can be present at concentrations ranging from fractions of up to 10 wt %. Many of these compounds are difficult to separate from acetonitrile as they either have very similar volatilities as acetonitrile within certain concentration ranges, or they form azeotropic mixtures with acetonitrile. A number of purification stages in this zone can be employed to separate acetonitrile from the other compounds present. One stage can be used to first separate a heavy boiling fraction of compounds from acetonitrile, light boilers and azeotrope formers. This stage can be designed to take advantage of even the smallest difference in relative volatility of the compounds to be separated to increase the purity of the acetonitrile stream. The light boiling fraction (207) from this stage containing the majority of the acetonitrile can then be fractionated in a further stage, e.g., in a second distillation zone (212), to produce a purified acetonitrile product stream (213) and a more volatile light boiler waste stream (214).

The organic waste stream (205) of the present disclosure which enters the first distillation zone comprises acetonitrile. In some embodiments, the organic waste stream comprises a relatively high content of acetonitrile. In one embodiment, the organic waste stream comprises acetonitrile in an amount ranging from 70 wt. % to 95 wt. % acetonitrile, based on the total weight of the feedstock stream, e.g., from 80 wt. % to 94 wt. %, from 85 wt. % to 93 wt. %, or from 87 wt. % to 92 wt. % acetonitrile, e.g., 90 wt. %. In terms of upper limits, the feedstock stream may comprise less than 95 wt. % acetonitrile, e.g., less than 94 wt. %, less than 93 wt. %, less than 92 wt. %, or less than 91 wt. % acetonitrile. In terms of lower limits, the feedstock stream may comprise greater than 70 wt. % acetonitrile, e.g., greater than 75 wt. %, greater than 80 wt. %, greater than 85 wt. %, greater than 86 wt. %, greater than 87 wt. %, greater than 88 wt. %, greater than 89 wt. %, acetonitrile, or greater than 90 wt. %, acetonitrile.

In some embodiments, the distillation column (206) operates in a range of 50 to 980 mbar, preferably at 100 to 300 mbar. In terms of lower limits, the first distillation column may operate at a pressure greater than 50 mbar, e.g., greater than 100, greater than 200, greater than 300 mbar, greater than 400 mbar, greater than 500 mbar, greater than 600 mbar, greater than 700 mbar, or greater than 800 mbar. In terms of upper limits, the first distillation column may operate a pressure less than 900 mbar, e.g., less than 800 mbar, less than 700 mbar, less than 600 mbar, less than 500 mbar, less than 400 mbar, less than 300 mbar, or less than 200 mbar.

Generally, distillation column (206) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (206) reflux ratio of step B is 2 to 8, e.g., 5.

The light boiling fraction (207) comprises about 90 wt. % to 99.5 wt. % acetonitrile. In some embodiments, the light boiling fraction comprises acetonitrile in an amount ranging from 96 wt. % to 99.5 wt. % acetonitrile, based on the total weight of the distillate, e.g., from 96.5 wt. % to 99.5 wt. %, from 97 wt. % to 99.5 wt. %, from 97.5 wt. % to 99.5 wt. %, or from 98 wt. % to 99.5 wt. % acetonitrile. In terms of upper limits, the feedstock stream may comprise less than 99.5 wt. % acetonitrile, e.g., less than 99 wt. %, less than 98.5 wt. %, less than 98 wt. %, less than 97.5 wt. %, less than 97 wt. %, less than 96.5 wt. %, less than 96 wt. %, less than 95.5 wt. %, or less than 95 wt. % acetonitrile. In terms of lower limits, the feedstock stream may comprise greater than 90 wt. % acetonitrile, e.g., greater than 91 wt. %, greater than 92 wt. %, greater than 93 wt %, greater than 94 wt %, greater than 95 wt %, greater than 95.5 wt %, greater than 96 wt %, greater than 96.5 wt %, greater than 97 wt %, greater than 97.5 wt %, greater than 98 wt %, greater than 98.5 wt %, or greater than 99 wt % acetonitrile.

Those impurities having a boiling point between the acetonitrile azeotrope boiling points and the acetonitrile boiling point, will be withdrawn together with the low boiling impurities, acetonitrile azeotropes and the acetonitrile. The light boiling fraction (207) is drawn from the upper portion from the column (206), e.g., from the top of the column. The azeotrope formers comprise toluene, ethanol, water and acrylonitrile. This fraction still contains high boiling hydrophobic impurities, such as toluene. This happens, because such high boiling hydrophobic impurities can form hydrophobic-hydrophobic interactions with acetonitrile and thereby are dragged over the column top during distillation. Hence, the fraction (207) withdrawn from the top of the column comprises acetonitrile as well as binary and ternary azeotropes, such as acetonitrile/diethylamine, acrylonitrile/water, acetonitrile/diisopropylamine, toluene/water/acrylonitrile, toluene/water/acetonitrile, diisopropylamine/water/acetonitrile, diethylamine/toluene/water, acetonitrile/water, acetonitrile/toluene, toluene/water, or ethanol/water, or mixtures thereof. Acetonitrile fraction (207) additionally comprises light boiling N-containing impurities, such as diethylamine, diisopropylamine, or acrylonitrile, or mixtures thereof.

The acetonitrile fraction (207) produced in step B may also comprise ethanol as an impurity. In some embodiments, the acetonitrile fraction (207) comprises a relatively low content of ethanol. In one embodiment, the light boiling fraction (207) comprises ethanol in an amount ranging from 0.5 wt. % to 3 wt. % ethanol, based on the total weight of the distillate (207), e.g., from 0.6 wt. % to 3 wt. %, from 0.7 wt. % to 3 wt. %, from 0.8 wt. % to 3 wt. % ethanol. In terms of upper limits, the feedstock stream may comprise less than 3 wt. % ethanol, e.g., less than 2.8 wt. %, less than 2.6 wt. %, less than 2.4 wt. %, less than 2.2 wt. %, less than 2 wt. %, less than 1.5 wt. % ethanol. In terms of lower limits, the feedstock stream may comprise greater than 0.5 wt. % ethanol, e.g., greater than 0.6 wt. %, greater than 0.7 wt. %, greater than 0.8 wt. %, greater than 0.9 wt. %, greater than 1 wt. %, greater than 1.2 wt. %, greater than 1.5 wt. %, greater than 1.7 wt. %, greater than 2 wt. %, or greater than 2.5 wt. % ethanol.

In some embodiments, the acetonitrile fraction (207) comprises toluene in an amount ranging from 0.2 wt. % to 5 wt. % toluene, e.g., from 0.5 wt. % to 5 wt. %, from 1 wt. % to 5 wt. %, from 1.5 wt. % to 5 wt. %, from 2 wt. % to 5 wt. %, from 2.5 wt. % to 5 wt. %, from 3 wt. % to 5 wt. %, from 3.5 wt. % to 5 wt. % toluene. In terms of upper limits, the light boiling acetonitrile fraction (207) may comprise less than 5 wt. % toluene, e.g., less than 4.5 wt. %, less than 4 wt. %, less than 3.5 wt. % toluene, less than 3 wt. %, less than 2.5 wt. %, less than 2 wt. %, less than 1.5 wt. %, less than 1 wt. %, or less than 0.5 wt. % toluene. In terms of lower limits, the light boiling acetonitrile fraction (207) may comprise greater than 0.2 wt. % toluene, e.g., greater than 0.5 wt. %, greater than 0.8 wt. %, greater than 1 wt. %, greater than 1.2 wt. %, greater than 1.5 wt. %, greater than 1.8 wt. %, greater than 2 wt. %, greater than 2.2 wt. %, greater than 2.5 wt. %, greater than 2.8 wt. %, greater than 3 wt. %, greater than 3.2 wt. %, greater than 3.5 wt. %, greater than 3.8 wt. %, greater than 4 wt. %, or greater than 4.5 wt. % toluene.

In some embodiments, the light boiling acetonitrile fraction (207) comprises acrylonitrile in an amount ranging from 1 to 200 ppm, e.g., from 5 to 180 ppm, from 50 to 150 ppm acrylonitrile. In terms of upper limits, the light boiling acetonitrile stream may comprise less than 200 ppm acrylonitrile, e.g., less than 180 ppm, less than 150 ppm, less than 120 ppm, less than 100 ppm, less than 75 ppm, less than 50 ppm, or less than 20 ppm acrylonitrile. In terms of lower limits, the light boiling acetonitrile fraction (207) may comprise greater than 1 ppm acrylonitrile, e.g., greater than 5 ppm, greater than 10 ppm, greater than 20 ppm, greater than 30 ppm, greater than 40 ppm, greater than 50 ppm, greater than 60 ppm, greater than 70 ppm, greater than 80 ppm, greater than 90 ppm, greater than 100 ppm, greater than 120 ppm, or greater than 150 ppm acrylonitrile.

In some embodiments, the light boiling acetonitrile fraction (207) comprises diethylamine in an amount ranging from 5 to 300 ppm, e.g., from 10 to 300 ppm, from 20 to 280 ppm from 50 to 250 ppm diethylamine. In terms of upper limits, the light boiling acetonitrile stream may comprise less than 300 ppm diethylamine, e.g., less than 280 ppm, less than 250 ppm, less than 230 ppm, less than 210 ppm, less than 200 ppm, less than 180 ppm, less than 150 ppm, less than 130 ppm, less than 100 ppm, or less than 50 ppm diethylamine. In terms of lower limits, the light boiling acetonitrile fraction (207) may comprise greater than 5 ppm diethylamine e.g., greater than 20 ppm, greater than 50 ppm, greater than 75 ppm, greater than 100 ppm, greater than 120 ppm, greater than 150 ppm, greater than 175 ppm, greater than 200 ppm, greater than 250 ppm diethylamine.

In some embodiments, the light boiling acetonitrile fraction (207) comprises diisopropylamine in an amount ranging from 10 to 600 ppm, e.g., from 50 to 550 ppm, from 75 to 500 ppm, from 100 to 400 ppm diisopropylamine. In terms of upper limits, the light boiling acetonitrile fraction (207) may comprise less than 600 ppm diisopropylamine, e.g., less than 550 ppm, less than 500 ppm, less than 450 ppm, less than 400 ppm, less than 350 ppm, less than 300 ppm, less than 250 ppm, less than 200 ppm, or less than 150 ppm diisopropylamine. In terms of lower limits, the light boiling acetonitrile fraction (207) may comprise greater than 10 ppm diisopropylamine, e.g., greater than 50 ppm, greater than 75 ppm, greater than 100 ppm, greater than 150 ppm, greater than 175 ppm, greater than 200 ppm, greater than 250 ppm, greater than 300 ppm greater than 350 ppm greater than 400 ppm greater than 550 ppm, or greater than 500 ppm diisopropylamine.

The light boiling acetonitrile fraction (207) leaves as a vapour and is passed through a condenser to produce a condensate. A portion of the condensate is then returned to the column as a reflux stream and the remainder is removed as a product, e.g., as a condensate from the upper portion, e.g., from the top of the column.

The heavier boiling organic impurities that are drawn as a bottoms product (208) from the distillation column, are considered as waste and are discarded. In some embodiments, the heavier boiling organic impurities comprise at least one of 1-methylimidazole, imidazole, 2,6-lutidine, acetic acid, dimethylformamide, ETT, PADS, pyridine and toluene, or mixtures thereof.

The light boiling acetonitrile fraction (203) of step A is then fed to the watering zone (209), where it is mixed with the light boiling acetonitrile fraction (207) from the organic purification step B along with water.

### Second Distillation Zone

Each of the light boiling streams (203, 207) from the aqueous and organic distillation steps A and B are fed to watering zone (209) wherein the light boiling streams (203, 207) from steps A and B are combined. The resultant acetonitrile composition may be further enriched with water before being fed into a further distillation column (212) (step C). The light boiling streams (203, 207) may be fed independently, e.g., one after the other, in any order or ratio ranging from 0-100%, or concurrently to watering zone (209).

Depending on the amount of water present from the first distillation zone from the aqueous waste stream (203), the water present in this stream may act as an entrainer, thus avoiding the need for the additional enrichment with water whereby the acetonitrile distillate (203) from the aqueous purification step is mixed with the acetonitrile distillate (207) form the organic purification step and water is added. Hence, in an alternative embodiment, the acetonitrile distillate (203) from the aqueous purification step is already enriched with enough water to produce a water enriched acetonitrile stream suitable for the separation of light boiling impurities and azeotrope formers. The water enriched acetonitrile stream (201a) as a direct result of mixing the acetonitrile distillates (203, 207) is then directly fed into the third distillation column (212) and process step D is carried out without the additional watering of step C.

The amount of water present in watering zone (209) by combining the purified aqueous and organic acetonitrile feeds (203, 207) can be readily determined by one of skill in the art. For example, the amount of water present can be determined by computer simulation based on the design specifications. The simulation can be conducted by well-known commercial simulation package, Aspen Plus (Aspen Technology, Inc., Massachusetts, USA), to simulate the feed composition to the column including the water content. In an embodiment, step C, e.g., step C1, further comprises the step of determining the amount of water present, e.g., by employing a process simulation software package, such as Aspen software, to determine if additional water is required to be added in watering zone (209).

The amount of water present by just adding the acetonitrile distillate of step A (203) and the acetonitrile distillate of step B (207) is less than 20 wt. %, e.g., less than 15 wt. %, e.g., less than 10 wt. %, e.g., about 8 wt %.

In an alternative embodiment, step C comprises feeding the acetonitrile distillate of step A (203) and the acetonitrile distillate of step B (207) to a watering zone (209) and combining therein and enriching said mixture with water. The amount of water added results in a water content of between 10 wt. % and 30 wt. % in the water enriched acetonitrile stream (210). The amount of water added is preferably greater than 0.24 times the amount of acetonitrile and impurities in the feed stream, but can be as low as 0.05 or as great as 0.50 times the total amount of acetonitrile and impurities present. Since the light boiling acetonitrile fraction (207) from the organic purification step B is mixed with light boiling acetonitrile fraction (203) from the aqueous purification of step A the amount of water that may be required for this step is lower compared to step C1 of the process of the second aspect.

The resultant water enriched acetonitrile stream (210, 201a) comprises about 60 to 90 wt. % acetonitrile, e.g., 76 wt. %. In one embodiment, the water enriched acetonitrile stream (210, 201a) comprises acetonitrile in an amount ranging from 65 wt. % to 90 wt. % acetonitrile, based on the total weight of the respective stream (210, 210a), e.g., from 65 wt. % to 85 wt. % acetonitrile. In terms of upper limits, the water enriched acetonitrile stream (210, 210a) may comprise less than 90 wt. % acetonitrile, e.g., less than 85 wt. %, less than 80 wt. %, less than 75 wt. %, or less than 70 wt. % acetonitrile. In terms of lower limits, the water enriched acetonitrile stream (210, 210a) may comprise greater than 65 wt. % acetonitrile, e.g., greater than 70 wt. %, greater than 75 wt. %, greater than 80 wt. %, or greater than 85 wt. % acetonitrile.

In an embodiment of step C, the light boiling streams (203, 207) from the aqueous and organic distillation steps A and B are also mixed with a recycled acetonitrile stream (221), wherein the recycled acetonitrile stream (221) is a by-product of a further processing step, e.g., a pressure swing distillation step, e.g., steps F1 and F2. The recycled acetonitrile stream (221) comprises a composition at or approaching the acetonitrile/water azeotropic composition at the operating conditions of the column (219) of step F2. The stream further comprises ethanol, which builds-up in the system. The amount of ethanol present will vary depending on the feed concentrations and the flow rate of this stream relative to the feed stream (213) entering the pressure swing distillation step, e.g., steps F1 and F2.

The recycled acetonitrile stream (221) may comprise acetonitrile in a range of 70 to 76 wt. %, e.g., 75 wt. %, ethanol in a range of 0 to 3 wt. %, e.g., 1.3 wt. %, and water in a range of 24 to 30 wt. %, e.g., 24 wt. % based on the total content of the stream.

Recycling 10 to 30% of this stream enables ethanol to be purged from the process in the form of an ethanol/water azeotrope. The recycling step ensures optimal removal of ethanol from the process. The resultant water enriched acetonitrile stream (210, 201a) is then fed into distillation column (212) and step D carried out.

The concept of recycling back to the oligonucleotide synthesis could employ uniquely a batch process to regenerate acetonitrile or a continuous process. The internal recycles in the regeneration process could also be done in batch if needed. The advantage of continuous is that the equipment will be much cheaper as much less storage volume will be required, the process can be automated and the volume required for storage is much less.

In some embodiments, water enriched acetonitrile stream (210, 201a) comprises about 60 to 90 wt. % acetonitrile. In one embodiment, the water enriched acetonitrile stream (210, 201a) comprises acetonitrile in an amount ranging from 65 wt. % to 90 wt. % acetonitrile, based on the total weight of the respective stream (210, 201a), e.g., from 65 wt. % to 85 wt. % acetonitrile. In terms of upper limits, the water enriched acetonitrile stream (210, 201a) may comprise less than 90 wt. % acetonitrile, e.g., less than 85 wt. %, less than 80 wt. %, less than 75 wt. %, or less than 70 wt. % acetonitrile. In terms of lower limits, the water enriched acetonitrile stream (210, 201a) may comprise greater than 65 wt. % acetonitrile, e.g., greater than 70 wt. %, greater than 75 wt. %, greater than 80 wt. %, or greater than 85 wt. % acetonitrile.

In some embodiments, the water enriched acetonitrile stream (210, 201a) comprises up to 1 wt. % ethanol. In a further embodiment, stream (210, 201a) comprises up to 20 ppm pyridine. In a further embodiment, stream (210, 201a) comprises up to 50 ppm toluene.

The acetonitrile enriched stream (210, 201a) is then fed into a distillation column (212). The acetonitrile enriched feed (210, 201a) is distilled and separated into an acetonitrile bottoms product (213) and a lower boiling fraction (214) comprising lower boiling azeotropes. All of the impurities in the feed are separated from the upper portion, e.g., from the top of the column (212) as binary or ternary azeotropes. The lower boiling azeotropic fraction (214) may be directed to organic waste. In an embodiment, at least a portion of said lower boiling azeotropic fraction (214) is first mixed, e.g., in a separate mixing zone (223), with other organic impurities, e.g., from a previous distillation step A (208), before being directed to organic waste.

In some embodiments, the distillation of step D operates in a range of 100-980 mbar, preferably at 900 to 980 mbar. In terms of lower limits, the distillation column (212) may operate at a pressure greater than 100 bar, e.g., greater than 200 mbar, greater than 300 mbar, greater than 400 mbar, greater than 500 mbar, greater than 600 mbar, greater than 700 mbar, or greater than 800 mbar. In terms of upper limits, the distillation column (212) may operate a pressure less than 980 mbar, e.g., less than 950 mbar, less than 900 mbar, or less than 800 mbar.

Generally, distillation column (212) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (212) reflux ratio of step D is 5 to 40, e.g., 30.

The lower boiling azeotropic fraction (214), which comprises at least one of acetonitrile/diethylamine, acetonitrile/diisopropylamine, toluene/water/acrylonitrile, toluene/water/acetonitrile, diisopropylamine/water/acetonitrile, acetonitrile/water, acetonitrile/toluene, toluene/water, and ethanol/water, or mixtures thereof. The lower boiling azeotropic fraction (214) may additionally comprise at least one N-containing compound, such as diethylamine, diisopropylamine, and acrylonitrile, or mixtures thereof.

The lower boiling azeotropic fraction is withdrawn from the upper portion, e.g., from the top of distillation column (212) as a vapour and is passed through a condenser to produce a condensate (214).

The acetonitrile produced as a bottoms product (213) comprises about 60 to 90 wt. % acetonitrile. In one embodiment, the acetonitrile bottoms product (213) comprises acetonitrile in an amount ranging from 65 wt. % to 90 wt. % acetonitrile, based on the total weight of the recovered acetonitrile bottoms product (213), e.g., from 65 wt. % to 85 wt. % acetonitrile. In terms of upper limits, the feedstock stream may comprise less than 90 wt. % acetonitrile, e.g., less than 85 wt. %, less than 80 wt. %, less than 75 wt. %, or less than 70 wt. % acetonitrile. In terms of lower limits, the feedstock stream may comprise greater than 65 wt. % acetonitrile, e.g., greater than 70 wt. %, greater than 75 wt. %, greater than 80 wt. %, or greater than 85 wt. % acetonitrile.

In some embodiments, the acetonitrile bottoms product (213) comprises up to 1 wt. % ethanol. In a further embodiment, the acetonitrile bottoms product (213) comprises up to 20 ppm pyridine. In a further embodiment, the acetonitrile bottoms product (213) comprises up to 50 ppm toluene.

The lower boiling azeotropic fraction (214) that is withdrawn from the upper portion, e.g., from the top of distillation column (212) is considered as waste and is discarded.

Thereby, it is ensured that a purified acetonitrile stream (213) is obtained and that the acetonitrile stream reaching the distillation column in the third distillation zone has a reduced water and impurity content and is as rich as possible in acetonitrile.

### Second and Third Aspects of the Present Disclosure

### Third Distillation Zone

The third distillation zone can be termed a de-watering zone and comprises the same steps, e.g., steps F1 and F2 for both the second and third aspects of the present disclosure unless specified otherwise. The acetonitrile streams (110, 216) from the previous two zones are combined in the feed to this zone. Along with acetonitrile and water, the feed (110, 216) contains residual quantities of other organic impurities in particular toluene, acrylonitrile and ethanol emanating from distillation zones 1 and 2. In distillation zone 3, the separation of water from acetonitrile can be achieved using a so-called pressure swing distillation system of two distillation columns (117, 120, 216, 219) operating at two different pressures (for example at 0.2 and 5 bar). This system takes advantage of the difference in azeotropic composition between the two pressures to produce a pure water waste and pure acetonitrile product stream from this zone. The impurities mentioned above form light boiling azeotropes with water and acetonitrile and an enriched stream of these impurities can be removed from the process at a separate location to the aqueous and acetonitrile product streams from the process. For the case that the water content in the product stream is not low enough an additional stage of separation can be added that could remove the residual water as an azeotrope or it could be removed by adsorption on to a stationary phase. The resulting acetonitrile has an assay purity, e.g., by gas chromatography of at least 99.90% and a water content below 30 ppm.

The reduction in water content of the acetonitrile stream fed to the third distillation zone, is achieved by the use of a pressure-swing distillation wherein a first distillation is performed at below atmospheric pressure, e.g., 0.20 bar. The percentage of water in the acetonitrile/water azeotrope is decreased at low pressure and increased at high pressure. It is preferable to perform them in separate columns. Hence, it is preferable to perform the lower pressure distillation in a first distillation column (117, 216) and then the higher pressure distillation in a second distillation column (120, 219). The second distillation is performed at a higher pressure than the first distillation, e.g., above atmospheric pressure, e.g., 5 bar.

Hence, in an embodiment, the process according to the first aspect of the present disclosure further comprises the steps of:
F1) introducing the acetonitrile enriched stream (110) into a fourth distillation column (117) and, performing a distillation at below atmospheric pressure to remove water therefrom, the acetonitrile being drawn as a vapour from said fourth distillation column and condensed to produce a fourth distillate (118), and water being produced as the fourth distillation column bottoms (119);
F2) introducing the fourth distillate (118) into a fifth distillation column (120) and, performing a second distillation at above atmospheric pressure, e.g., 5 bar, to produce lower boiling fraction (122) being drawn via the upper portion of the fifth distillation column (120) and acetonitrile being produced as the fifth distillation column bottoms (121).

The acetonitrile drawn from distillation column in step F2 (121) is more than 99.90% pure when measured by GC based on the total content of the acetonitrile stream (121).

Preferably, the acetonitrile drawn from distillation column in step F2 (121) is more than 99.90% pure and comprises less than 30 ppm water when measured by GC.

In an embodiment, the process according to the second aspect of the present disclosure further comprises the steps of:
F1) introducing the acetonitrile bottoms product (213) from the distillation of step D into a fourth distillation column (216) and, performing a distillation at below atmospheric pressure (e.g., 0.2 bar) to remove water therefrom, the acetonitrile being drawn as a vapour from said fourth distillation column (216) and condensed to produce a fourth distillate (217), and water being produced as the fourth distillation column bottoms (218);
F2) introducing the fourth distillate (217) into a fifth distillation column (219) and, performing a second distillation at above atmospheric pressure, e.g., 5 bar, to produce lower boiling fraction (221) being drawn via the upper portion of the fifth distillation column (219) and acetonitrile being produced as the fifth distillation column bottoms (220).

The acetonitrile drawn from said column in step F2 (220) is more than 99.90% pure when measured by GC based on the total content of the acetonitrile stream (220). Preferably, the acetonitrile drawn from distillation column in step F2 (220) is more than 99.90% pure and comprises less than 30 ppm water when measured by GC.

In an embodiment the acetonitrile bottoms product (213) of step D is first fed into a further mixing zone (215), whereby it is mixed with a recycled acetonitrile stream (221) (step E), wherein the recycled acetonitrile stream (221) is a by-product of a further processing step, e.g., a pressure swing distillation step. In an embodiment, the recycled acetonitrile stream (221) is a by-product of step F2, e.g., it is the lower boiling fraction (221) from distillation step F2.

In an embodiment, the distillation of step F1 for processes according to the second and third aspects of the present disclosure operates in a range of 50 to 980 mbar, preferably between 50 to 200 mbar, e.g., 200 mbar. In terms of lower limits, the distillation of step F1 may operate at a pressure greater than 50 mbar, e.g., greater than 100 mbar, greater than 150 mbar, or greater than 175 mbar, or greater than 200 mbar. In terms of upper limits, the distillation of step F1 may operate a pressure less than atmospheric pressure, e.g., less than 980 mbar, less than 900 mbar, or less than 800 mbar, less than 700 mbar less than 600 mbar less than 500 mbar, less than 400 mbar, less than 300 bar, or less than 200 mbar.

Generally, distillation column (117, 216) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (117, 216) reflux ratio of step F1 is 0.05 to 1, e.g., 0.1.

The lighter boiling fraction (118, 217) in step F1 comprising acetonitrile as well as an azeotropic acetonitrile composition plus excess water or approaching the azeotropic composition, and impurities such as ethanol is withdrawn from the upper portion, e.g., from the top of the column (117, 216) as a condensate. The water withdrawn as a bottoms product (119, 218) is considered as waste and is discarded.

In some embodiments, the light boiling acetonitrile fraction (118, 217) drawn via the upper portion, e.g., from the top of the distillation column (117, 216) in step F1 comprises acetonitrile in an amount ranging from 75 wt. % to 92 wt. % acetonitrile, based on the total weight of the acetonitrile fraction. In terms of upper limits, the light boiling acetonitrile fraction (118, 217) may comprise less than 92 wt. % acetonitrile, e.g., less than 90 wt. %, less than 85 wt. %, less than 80 wt. % acetonitrile. In terms of lower limits, the light boiling acetonitrile fraction (118, 217) may comprise greater than 75 wt. % acetonitrile, e.g., greater than 80 wt. %, greater than 85 wt. %, or greater than 90 wt. % acetonitrile.

In some embodiments, the light boiling acetonitrile fraction (118, 217) of step F1 comprises water in an amount ranging from 8 wt. % to 28 wt. % water, based on the total weight of the acetonitrile fraction. In terms of upper limits, the light boiling acetonitrile fraction (118, 217) may comprise less than 28 wt. % water, e.g., less than 25 wt. %, less than 23 wt. %, less than 20 wt. %, less than 15 wt. %, or less than 10 wt. % water. In terms of lower limits, the light boiling acetonitrile fraction (118, 217) may comprise greater than 8 wt. % water, e.g., greater than 10 wt. %, greater than 15 wt. %, greater than 20 wt. %, or greater than 25 wt. % water.

In some embodiments, the light boiling acetonitrile fraction (118, 217) comprises ethanol in an amount ranging from 0.2 wt. % to 3 wt. % ethanol, based on the total weight of the acetonitrile fraction. In terms of upper limits, the light boiling acetonitrile fraction (118, 217) may comprise less than 3 wt. % ethanol, e.g., less than 2 wt. %, less than 1.5 wt. %, less than 1 wt. %, less than 0.8 wt. %, less than 0.5 wt. %, or less than 0.3 wt. % ethanol. In terms of lower limits, the light boiling acetonitrile fraction (118, 217) may comprise greater than 0.2 wt. % ethanol, e.g., greater than 0.5 wt. %, greater than 1 wt. %, or greater than 2 wt. % ethanol.

Thereby it is ensured that the stream (118, 217) reaching the higher pressure distillation column (120, 219) has a reduced water content and is as rich as possible in acetonitrile. During the distillation of step F2, performed at a higher pressure than the distillation of step F1, the water content of the azeotrope increases and reaches approximately 23% by weight at 5 bar. Hence, the remaining water can be distilled away in the form of an acetonitrile/water azeotrope (122, 221). This azeotrope (122) is preferably recycled to the acetonitrile feedstock in the first distillation zone of the process according to the first aspect, e.g., for the distillation of step D1, or more preferably via the watering zone (109), e.g., in step C1.

According to the second aspect of the disclosure, the azeotrope (221) is preferably recycled to the acetonitrile feedstock in the second distillation zone of the process, e.g., for the distillation of step D, or more preferably via the watering zone (209) whereby the light boiling streams (203, 207) from the aqueous and organic distillation steps A and B are combined, e.g., in step C.

Thus, in an embodiment, any impurities present in the acetonitrile stream (118, 217), which form light boiling azeotropes with water and acetonitrile are withdrawn from the upper portion, e.g., from the top of distillation column (120, 219) may be recycled back to step D1, e.g., via step C1, or step D, e.g., via step C along with any remaining acetonitrile and water and may be fed back into the distillation processes of the present disclosure.

In a further embodiment of the first aspect, said impurities (122) from the pressure-swing distillation steps, e.g., F1 and F2, are recycled back to step D1 via the watering zone (109) of step C1 where the feed is enriched with water to produce lower boiling azeotropes relative to acetonitrile, and introduced into step D1.

In a separate embodiment of the first aspect, said impurities (122, 221) from the pressure-swing distillation steps, e.g., F1 and F2, may be purged and sent to waste.

In some embodiments, the distillation of step F2 for processes according to the second and third aspects of the present disclosure operates at above atmospheric pressure or in a range of 3-8 bar, preferably at 5 bar. In terms of lower limits, the distillation of step F2 may operate at a pressure greater than 3 bar, e.g., greater than 4, greater than 4.5, or greater than 5 bar. In terms of upper limits, the distillation of step F2 may operate a pressure less than 8 bar, e.g., less than 7 bar, less than 6 bar, or less than 5.5 bar.

Generally, distillation column (120, 219) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In an embodiment of the present disclosure the distillation column (120, 219) reflux ratio of step F2 is 0.05 to 1, e.g., 0.25.

The acetonitrile drawn as a bottoms product (121, 220) from the higher pressure distillation column of step F2 is more than 99.90% pure (area % measurement by GC analysis) based on the total content of the acetonitrile stream (121, 220). Preferably, the acetonitrile bottoms product (121, 220) also has a water content below 30 ppm.

In some embodiments, the acetonitrile bottoms product (121, 220) comprises acetonitrile in an amount ranging from 99.90% to 99.99% (area %) acetonitrile, when measured by gas chromatography analysis. In terms of upper limits, the acetonitrile bottoms product (121, 220) may comprise less than 99.99% acetonitrile, e.g., less than 99.98%, less than 99.95%, less than 99.92% acetonitrile. In terms of lower limits, the acetonitrile bottoms product (121, 220) may comprise greater than 99.90% acetonitrile, e.g., greater than 99.92%, greater than 99.95%, or greater than 99.98% acetonitrile. Preferably, the acetonitrile bottoms product (121, 220) also has a water content below 30 ppm.

In some embodiments the acetonitrile bottoms product (121, 220) comprises water in an amount ranging from 10 to 30 ppm water, based on the total weight of the acetonitrile fraction.

In terms of upper limits, the acetonitrile bottoms product (121, 220) may comprise less than 30 ppm water, e.g., less than 28 ppm, less than 25 ppm, less than 23 ppm, less than 20 ppm less than 18 ppm, less than 15 ppm, or less than 12 ppm water. In terms of lower limits, the acetonitrile bottoms product (121, 220) may comprise greater than 10 ppm water, e.g., greater than 12 ppm, greater than 15 ppm, greater than 20 ppm water.

In some embodiments, the acetonitrile bottoms product (121, 220) may comprise relatively small amounts of organic impurities. In some embodiments, the acetonitrile bottoms product (121, 220) may comprise residual ethanol, e.g., as an impurity, in an amount ranging from 1 to 10 ppm ethanol, based on the total weight of the acetonitrile fraction. In terms of upper limits, the feedstock stream may comprise less than 10 ppm ethanol, e.g., less than 9 ppm, less than 8 ppm, less than 7 ppm, less than 6 ppm, less than 5 ppm, less than 4 ppm, less than 3 ppm, or less than 2 ppm ethanol. In terms of lower limits, the feedstock stream may comprise greater than 1 ppm ethanol, e.g., greater than 3 ppm, greater than 5 ppm, or greater than 8 ppm ethanol.

In an embodiment, the acetonitrile bottoms product (121, 220) comprises pyridine in an amount ranging from 1 to 5 ppm pyridine, based on the total weight of the acetonitrile fraction. In terms of upper limits, the acetonitrile bottoms product may comprise less than 5 ppm pyridine, e.g., less than 4 ppm, less than 3 ppm, or less than 2 ppm pyridine. In terms of lower limits, the feedstock stream may comprise greater than 1 ppm pyridine, e.g., greater than 2 ppm, greater than 3 ppm, or greater than 4 ppm pyridine.

In some embodiments, the acetonitrile bottoms product (121, 220) may comprise toluene as an impurity. In an embodiment, the acetonitrile bottoms product comprises toluene in an amount ranging from 1 to 10 ppm toluene, based on the total weight of the acetonitrile fraction toluene. In terms of upper limits, the acetonitrile bottoms product may comprise less than 10 ppm toluene, e.g., less than 8 ppm, less than 7 ppm, less than 6 ppm, less than 5 ppm, less than 4 ppm, less than 3 ppm, less than 2 ppm toluene. In terms of lower limits, the acetonitrile bottoms product may comprise greater than 1 ppm toluene, e.g., greater than 2 ppm, greater than 3 ppm, greater than 4 ppm, greater than 5 ppm, greater than 6 ppm, greater than 7 ppm, greater than 8 ppm, greater than 9 ppm toluene.

In order to be reused in oligonucleotide manufacture an acetonitrile purity of at least 99.90% must be achieved. Furthermore, a water content less than 30 ppm is desirable. Otherwise, the impurity level is too high and is not suitable for reuse in industrial or manufacturing oligonucleotide processes.

Therefore, in the case that the water content in the product stream from the pressure-swing distillation steps is not low enough an additional stage of separation can be added that could remove the residual water as an azeotrope or it could be removed by adsorption on to a stationary phase, e.g., molecular sieves.

Therefore, in a further embodiment of the disclosure, the processes according to the second or third aspects of the present disclosure additionally comprises the step of feeding the acetonitrile bottoms product (121, 220) of step F2 to a water adsorption zone (124, 225) and contacting said acetonitrile bottoms product (121, 220) with a water reducing adsorbent to produce a highly purified acetonitrile that exits the adsorption zone. The water reducing adsorbent may be molecular sieves. Further details and embodiments for the adsorbent(s) are described at [0033] to [0038] of WO02015126713A1. In a preferred embodiment, the molecular sieves have a pore size of 3 Angstroms. Since water has a size less than this and acetonitrile has a size greater than this, a pore size of 3 Angstroms is very efficient for separation. The acetonitrile leaving the adsorption zone has purity of at least 99.90%, e.g., when measured by gas chromatography, and has a water content of below 30 ppm.

### Fourth Aspect of the Present Disclosure

In a fourth aspect, there is provided a process for recovering and/or purifying acetonitrile from waste acetonitrile (301, 302) generated during oligonucleotide synthesis, the process comprising the steps:
A′) introducing an organic waste feedstock (301) comprising acetonitrile, a first set of organic impurities having a lower boiling temperature than acetonitrile and a second set of organic impurities having a boiling temperature greater than acetonitrile, and an aqueous waste feedstock (302) comprising acetonitrile a first set of aqueous impurities having a lower boiling temperature than acetonitrile and a second set of aqueous impurities having a boiling temperature greater than acetonitrile, into a first mixing zone (303), and combining therein, to form an acetonitrile waste feedstock comprising said impurities; or
introducing an organic waste feedstock (301) comprising acetonitrile, a first set of organic impurities having a lower boiling temperature than acetonitrile and a second set of organic impurities having a boiling temperature greater than acetonitrile, into a first mixing zone (303), and enriching with water, to produce a water enriched acetonitrile stream comprising said impurities;
B′) introducing the acetonitrile waste feedstock or water enriched acetonitrile stream into a first distillation column (304) and separating the acetonitrile and the first set of impurities having a lower boiling temperature than acetonitrile from the second set of impurities having a boiling temperature greater than acetonitrile, the acetonitrile and first set of impurities being drawn as a vapour from said first distillation column and condensed to produce a first distillate (306), the second set of impurities being produced as the first distillation column bottoms (305);
C′) optionally introducing the first distillate (306) comprising acetonitrile and low boiling impurities, into a watering zone (307) and enriching with water to produce a water enriched acetonitrile stream (307a); and
D′) introducing the water enriched acetonitrile stream (306, 307a) into a second distillation column (308) and separating the acetonitrile from the low boiling impurities, the acetonitrile being produced as the second distillation column bottoms (309);
such that recovered and/or purified acetonitrile is obtained.

The second distillation column bottoms (309) produced as a result of the second distillation step D′ comprises an acetonitrile/water azeotrope.

In order to reduce the water content of the acetonitrile product to less than 30 ppm, a further processing step may be employed. In an embodiment of the fourth aspect of the present disclosure, the process further comprises the step:
E′) introducing the second distillation column bottoms product (309) of step D′ into a third distillation column (311) and separating the acetonitrile from the acetonitrile/water azeotrope, the acetonitrile being produced as the third distillation column bottoms (312).

The introduction of the organic and aqueous waste feedstocks of the alternative embodiment of step A′ may be executed independently, e.g., one after the other, in any order or ratio ranging from 0-100%, or concurrently. The steps may be started concurrently as the waste streams will be produced at the same rate from the main synthesis/purification sections of the process, although this is not essential.

The process of the fourth aspect of the disclosure incorporates a pressure-swing distillation technique across all three columns to recover acetonitrile with a water content of less than 30 ppm. The system comprises three distillation columns (304, 308, 311) which operate at low-pressure, e.g., 20 to 500 mbar, e.g., 200 mbar, mid-pressure, e.g., 200 mbar to 2 bar, e.g., 960 mbar, and high-pressure, e.g., 2 bar to 5 bar, e.g., 5 bar.

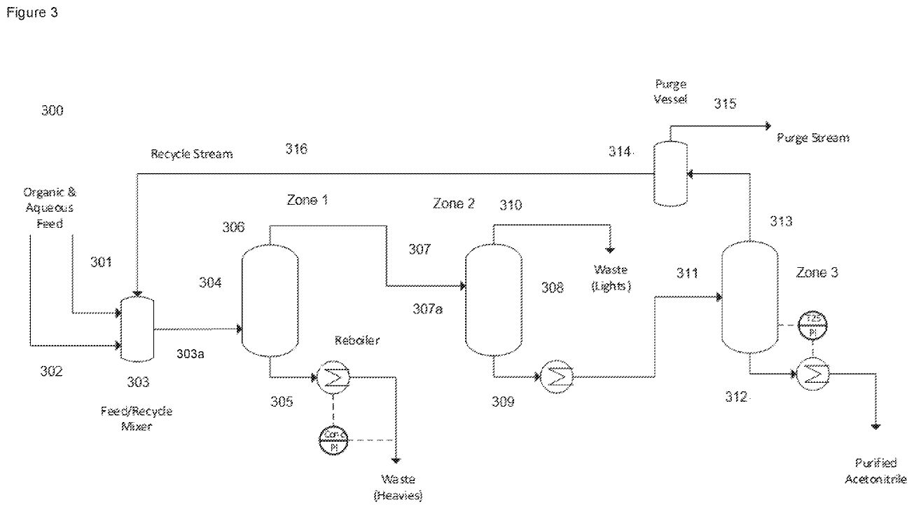

The distillation step B′ takes place in the so-called first distillation zone (FIG. 3, zone 1). In one embodiment, the acetonitrile waste comprising aqueous and organic impurities (303) entering this zone is separated into a stream (306) enriched in acetonitrile and comprising water and ethanol as well as lighter boiling organic impurities. The acetonitrile-water azeotrope is distilled overhead and excess water along with higher boiling components which do not form binary or ternary azeotropes are removed from the base of the column (305). The heavy components include acetic acid, acetic anhydride, N-dimethylformamide, 1-methylimidazole, dichloroacetic acid, 2,6-lutidine, 5-ethylthio-1H-tetrazole and pyridine. Two components which have individual higher boiling points compared to acetonitrile but are not separated in this step are toluene and diisopropylamine, as they both form low-boiling binary and ternary azeotropes with acetonitrile and water. This zone is operated to maximize the yield of acetonitrile in the form of an azeotrope while removing as much excess water as possible from the process.

In step B′, the acetonitrile fraction is drawn from the upper portion, e.g., from the top of the distillation column (304). The acetonitrile fraction leaves as a vapour and is passed through a condenser to produce a condensate (306). A portion of the condensate is then returned to the column as a reflux stream and the remainder is removed as a product. The acetonitrile fraction (306) comprises acetonitrile and light boiling impurities, such as an acetonitrile/water azeotrope as well as binary and ternary azeotropes comprising toluene and diisopropylamine. The acetonitrile fraction may additionally comprise ethanol. Heavy impurities, e.g., having a boiling temperature greater than acetonitrile, are drawn as a distillation column bottoms (305). The heavy boiling impurities (305) comprise water, non-volatile salts and organic residue. The bottoms product (305) is considered as waste and is discarded.

For all columns utilized in the processes of the present disclosure, a heating source may be provided at the base of the column to evaporate a fraction of acetonitrile and light boilers still present at the base of the column, and to provide a vapour flow for heat exchange further up the column. In an embodiment, the heating source is a reboiler.

The process step C′ takes place in the so-called second distillation zone (FIG. 3, zone 2).

Step C′ comprises introducing the distillate (306) from step B′ into a watering zone (307) and enriching said distillate (307) with water to produce a water enriched acetonitrile stream. In order to separate azeotrope formers with acetonitrile, such as toluene or diisopropylamine, an entrainer (e.g., water) can be added to the feed that will produce lower boiling azeotropes with these compounds relative to acetonitrile making them easier to separate from acetonitrile. To inventors' surprise, water, unexpectedly, reduces the loss of acetonitrile from the process as the azeotropes are largely impurity and water rich with lower acetonitrile content. The amount of water added relative to acetonitrile may be in excess of 0.24 w/w, but can be anywhere between 0.05 and 0.40 w/w or greater based on the amount of acetonitrile and impurities present in the stream. Since the light boiling acetonitrile fraction (306) from step B′ is the result of mixing the aqueous and organic waste streams in step A′ the amount of water required for this step is lower compared to step C1 of the process of the first aspect. The amount of water added is relative to the amount of acetonitrile and impurities which ensures that column operation is stable.

The lower boiling azeotropes of the water enriched acetonitrile stream of step C′ comprise at least one of acetonitrile, water, ethyl acetate, toluene, ethanol, diethylamine, diisopropylamine, and acrylonitrile, or mixtures thereof. For example, the lower boiling azeotropes of step C′ comprise at least one of ethyl acetate/water, ethyl acetate/acetonitrile, ethyl acetate/water/acetonitrile, acetonitrile/water, acrylonitrile/water, acetonitrile/toluene, acetonitrile/diethylamine, acetonitrile/diisopropylamine, toluene/water, ethanol/water, toluene/water/acrylonitrile, toluene/water/acetonitrile, diisopropylamine/water/acetonitrile, diethylamine/toluene/water, or mixtures thereof.

Step C′ is optional since, depending on the amount of water present in the first mixing zone (303) from the aqueous waste stream (302), the water present in the mixing zone (303) may be sufficient to act as an entrainer, thus avoiding the need for the additional step C′ whereby the acetonitrile distillate (306) is enriched with water (307) to produce a water enriched acetonitrile stream. Thus, in an alternative embodiment, the lower boiling azeotropes present in step C′ will be present in the acetonitrile stream (306) from step B′, in which case, mixing zone (303) functions as a watering zone.

The amount of water present in mixing zone (303) by combining the aqueous and organic waste streams (301, 302) can be readily determined by one of skill in the art. For example, the amount of water present can be determined by computer simulation based on the design specifications. The simulation can be conducted by well-known commercial simulation package, Aspen Plus (Aspen Technology, Inc., Massachusetts, USA), to simulate the feed composition to the column including the water content. In an embodiment, step A′ further comprises the step of determining the amount of water present in the acetonitrile waste feedstock, e.g., by employing a process simulation software package, such as Aspen software, to determine if additional water is required to be added in watering zone (307).

In an alternative embodiment, the organic waste feedstock (301) comprising acetonitrile, and said organic impurities, is fed into watering zone (303), and directly enriched with water, to produce a water enriched acetonitrile stream comprising said impurities without the need for the additional aqueous waste stream feed.

The acetonitrile stream (306) is then directly fed into the second distillation column (308) and process steps D′ and E′ are carried out.

The water enriched acetonitrile feed is distilled and separated into an acetonitrile bottoms product (309) and a top fraction (310) comprising lower boiling azeotropes, the acetonitrile bottoms product (309) having a greater acetonitrile concentration than said water enriched acetonitrile stream from step C′. The lower boiling azeotropes (310) are drawn from the upper portion, e.g., from the top of the distillation column (308). Such lower boiling azeotropic fraction may be directed to waste. Thereby it is ensured that the resultant purified acetonitrile product or the acetonitrile stream reaching the column, in which the distillation of step E′ is performed, has a reduced water content and is as rich as possible in acetonitrile.

The distillation step E′ comprises introducing the acetonitrile bottoms product from step D′ into a third distillation column (311), distilling said acetonitrile bottoms product (309) to remove any remaining water or organic impurities, such as ethanol, therefrom to produce a top fraction (313) comprising lower boiling azeotropes relative to acetonitrile, and an acetonitrile bottoms product (312). Hence, the remaining water can be distilled away in the form of an acetonitrile/water azeotrope. Such lower boiling azeotropic fraction (313) may be recycled back into step A′ (316) and fed into the first mixing zone (303). In an embodiment, at least a portion of said lower boiling azeotropic fraction (313) is first purged (314), i.e. removed from the system, before being recycled to step A′ (316). In another embodiment, the lower boiling azeotropic fraction (313) is purged (314) and sent to waste (315).

The acetonitrile (312) produced in step E′ can be produced in a purity of at least 99.90% with less than 30 ppm water. Yields in the order of 85% were calculated.

Advantageously, the above process steps A′ to D′ provide an improved acetonitrile recovery and/or purification process. Such process can successfully remove high boiling hydrophobic organic and inorganic aqueous impurities from acetonitrile waste streams, e.g., those produced during oligonucleotide synthesis. The acetonitrile produced according to steps A′ to D′ is 99.90% (area %) pure according to gas chromatography analysis. Such recovered acetonitrile can be conveniently reused directly in oligonucleotide manufacture.

The amount of acetonitrile that can be regenerated in the process employing steps A′ to E′ represents between 70 and 90% of the total acetonitrile consumed in the oligonucleotide synthesis process.

In the case that the water content in the acetonitrile product stream (312) from step E′ is not low enough, an additional stage of separation can be added that could remove the residual water as an azeotrope or it could be removed by adsorption on to a stationary phase, e.g., molecular sieves. The details of this additional drying step are the same as those described herein for the first, second and third aspects of the present disclosure.

The processes of the present disclosure are also used to purify an acetonitrile waste stream, e.g., from oligonucleotide synthesis to generate purified acetonitrile suitable for reuse in various industrial or manufacturing processes, in particular in oligonucleotide manufacture. In some embodiments, the purified acetonitrile obtained according to the processes disclosed herein is suitable for reuse as a between-step wash solution in an oligonucleotide synthesis conducted with between-step acetonitrile washes. In some embodiments, the purified acetonitrile is suitable as a reaction solvent during oligonucleotide synthesis steps. In an embodiment, the purified acetonitrile is least 99.90% pure, e.g., when measured by gas chromatography. In a preferred embodiment, the purified acetonitrile is least 99.90% pure and contains less than 30 ppm water, e.g., when measured by gas chromatography.

In an embodiment, the waste acetonitrile according to any one of the first, second, third and fourth aspects of the disclosure is of industrial scale. In an embodiment, the amount of waste acetonitrile is at least 10 kg, preferably at least 100 kg, at least 1000 kg or at least 10,000 kg. In a further embodiment, the acetonitrile is recovered at the gram or kilogram scale, or greater.

In a further embodiment, the process according to any one of the first, second, third and fourth aspects of the disclosure is carried out in batch. In another embodiment, the process according to any one of the first, second, third and fourth aspects of the disclosure is carried out on a continuous basis.

In a further aspect, provided herein is a process for synthesizing an oligonucleotide, the process comprising recovering and/or purifying acetonitrile by:
A*) introducing an organic waste feedstock (105, 205) comprising acetonitrile, a first set of organic impurities having a lower boiling temperature than acetonitrile and a second set of organic impurities having a boiling temperature greater than acetonitrile, into a distillation column (106, 206) and separating the acetonitrile and first set of organic impurities from the second set of organic impurities, the acetonitrile and first set of organic impurities being drawn as a vapour from said distillation column and condensed to produce a distillate (107, 207), the second set of organic impurities being produced as the second distillation column bottoms (108, 208);
B*) introducing the distillate (107, 207) into a watering zone (109, 209) to produce a water enriched acetonitrile stream (111, 210, 210a);
C*) introducing the water enriched acetonitrile stream (111, 210, 210a) into a second distillation column (112, 212) and separating the first set of organic impurities from the acetonitrile, the acetonitrile being produced as the second distillation column bottoms (113, 213);
such that recovered and/or purified acetonitrile is obtained;
D*) optionally further processing the second distillation column bottoms (113, 213) to reduce the water content of the acetonitrile to less than 30 parts per million; and
E*) using at least a portion of the recovered and/or purified acetonitrile in a process for synthesizing an oligonudeotide and/or washing an oligonucleotide or support-bound oligonucleotide with a portion of the recovered and/or purified acetonitrile.

The recovered acetonitrile may be re-used in oligonucleotide manufacture immediately or stored until ready for use.

In a further aspect, provided herein is a process for synthesizing an oligonucleotide, the process comprising recovering and/or purifying acetonitrile by:
A1) introducing an aqueous waste feedstock (101) comprising acetonitrile, a first set of aqueous impurities having a lower boiling temperature than acetonitrile and a second set of aqueous impurities having a boiling temperature greater than acetonitrle, into a first distillation column (102) and separating the acetonitrle and first set of impurities from the second set of impurities, the acetonitrile and first set of impurities being drawn as a vapour from said first distillation column and condensed to produce a first distillate (103), the second set of impurities being produced as the first distillation column bottoms (104);
B1) introducing an organic waste feedstock (105) comprising acetonitrile, a first set of organic impurities having a lower boiling temperature than acetonitrile and a second set of organic impurities having a boiling temperature greater than acetonitrile, into a second distillation column (106) and separating the acetonitrile and first set of organic impurities from the second set of organic impurities, the acetonitrile and first set of organic impurities being drawn as a vapour from said second distillation column and condensed to produce a second distillate (107), the second set of organic impurities being produced as the second distillation column bottoms (108);
C1) introducing the second distillate (107) into a watering zone (109) and enriching with water to produce a water enriched acetonitrile stream (111);
D1) introducing the water enriched acetonitrile stream (111) into a third distillation column (112) and separating the first set of organic impurities from the acetonitrile, the acetonitrile being produced as the third distillation column bottoms (113); and
E1) feeding the first distillate (103) of step A1 and the third distillation column bottoms (113) of step D1 to a mixing zone (116) and combining therein, to produce an acetonitrile enriched stream (110);
such that recovered and/or purified acetonitrile is obtained;
F1a) optionally further processing the acetonitrile enriched stream (110) to reduce the water content of the acetonitrile to less than 30 parts per million; and
G1) using at least a portion of the recovered and/or purified acetonitrile in a process for synthesizing an oligonucleotide and/or washing an oligonucleotide or support-bound oligonucleotide with a portion of the recovered and/or purified acetonitrile.

The recovered acetonitrile may be re-used in oligonucleotide manufacture immediately or stored until ready for use.

In a further aspect, there is provided a process for synthesizing an oligonucleotide, the process comprising recovering and/or purifying acetonitrile by:
A) introducing an aqueous waste feedstock (201) comprising acetonitrile, a first set of aqueous impurities having a lower boiling temperature than acetonitrile and a second set of aqueous impurities having a boiling temperature greater than acetonitrile, into a first distillation column (202) and separating the acetonitrile and first set of impurities from the second set of impurities, the acetonitrile and first set of impurities being drawn as a vapour from said first distillation column and condensed to produce a first distillate (203), the second set of impurities being produced as the first distillation column bottoms (204);
B) introducing an organic waste feedstock (205) comprising acetonitrile, a first set of organic impurities having a lower boiling temperature than acetonitrile and a second set of organic impurities having a boiling temperature greater than acetonitrile, into a second distillation column (206) and separating the acetonitrile and first set of organic impurities from the second set of organic impurities, the acetonitrile and first set of organic impurities being drawn as a vapour from said second distillation column and condensed to produce a second distillate (207), the second set of organic impurities being produced as the second distillation column bottoms (208);
C) feeding the first distillate of step A (203) and the second distillate of step B (207) to a watering zone (209) and
C1) combining therein to produce a water enriched acetonitrile stream (210a), or
C2) combining therein and enriching with water to produce a water enriched acetonitrile stream (210); and
D) introducing the water enriched acetonitrile stream (210, 201a) into a third distillation column (212) and separating the first sets of impurities from the acetonitrile, the acetonitrile being produced as the third distillation column bottoms (213);
such that recovered and/or purified acetonitrile is obtained;
E) optionally further processing the third distillation column bottoms (213) to reduce the water content of the acetonitrile to less than 30 parts per million; and
F) using at least a portion of the recovered and/or purified acetonitrile in a process for synthesizing an oligonucleotide and/or washing an oligonucleotide or support-bound oligonucleotide.

The recovered and/or purified acetonitrile may be re-used in oligonucleotide manufacture immediately or stored until ready for use.

The further processing of the acetonitrile enriched stream (110) or the third distillation column bottoms (213) to reduce the water content of the acetonitrile to less than 30 parts per million may comprise the step of introducing the acetonitrile enriched stream (110) or the third distillation column bottoms (213) into a fourth distillation column (117, 216), wherein the water content of the acetonitrile enriched stream is reduced by use of a pressure-swing distillation to recover acetonitrile having a water content of less than 30 parts per million, as described above.

In an embodiment, the pressure-swing distillation comprises the steps:
F1) introducing the acetonitrile enriched stream (110) or the third distillation column bottoms (213) into a fourth distillation column (117, 216) and, performing a distillation at below atmospheric pressure to remove water therefrom, the acetonitrile being drawn as a vapour from said fourth distillation column and condensed to produce a fourth distillate (118, 217), and water being produced as the fourth distillation column bottoms (119, 218);
F2) introducing the fourth distillate (118, 217) into a fifth distillation column (120, 219) and, performing a second distillation at above atmospheric pressure, e.g., 5 bar, to produce lower boiling fraction (122, 221) being drawn via the upper portion of the fifth distillation column (120, 219) and acetonitrile being produced as the fifth distillation column bottoms (121, 220), as described above.

In yet a further aspect, there is provided a process for synthesizing an oligonucleotide, the process comprising recovering and/or purifying acetonitrile by:
A′) introducing an organic waste feedstock (301) comprising acetonitrile, a first set of organic impurities having a lower boiling temperature than acetonitrile and a second set of organic impurities having a boiling temperature greater than acetonitrile, and an aqueous waste feedstock (302) comprising acetonitrile a first set of aqueous impurities having a lower boiling temperature than acetonitrile and a second set of aqueous impurities having a boiling temperature greater than acetonitrile, into a first mixing zone (303), and combining therein, to form an acetonitrile waste feedstock comprising said impurities; or
introducing an organic waste feedstock (301) comprising acetonitrile, a first set of organic impurities having a lower boiling temperature than acetonitrile and a second set of organic impurities having a boiling temperature greater than acetonitrile, into a first mixing zone (303), and enriching with water, to produce a water enriched acetonitrile stream comprising said impurities;
B′) introducing the acetonitrile waste feedstock or water enriched acetonitrile stream into a first distillation column (304) and separating the acetonitrile and the first set of impurities having a lower boiling temperature than acetonitrile from the second set of impurities having a boiling temperature greater than acetonitrile, the acetonitrile and first set of impurities being drawn as a vapour from said first distillation column and condensed to produce a first distillate (306), the second set of impurities being produced as the first distillation column bottoms (305);
C′) optionally introducing the first distillate (306) comprising acetonitrile and low boiling impurities, into a watering zone (307) and enriching with water to produce a water enriched acetonitrile stream (307a);
D′) introducing the water enriched acetonitrile stream (306, 307a) into a second distillation column (308) and separating the acetonitrile from the low boiling impurities, the acetonitrile being produced as the second distillation column bottoms (309);
such that recovered and/or purified acetonitrile is obtained;
E) optionally introducing the second distillation column bottoms product (309) of step D′ into a third distillation column (311) and separating the acetonitrile from the acetonitrile/water azeotrope, the acetonitrile being produced as the third distillation column bottoms (312);
and
F) using at least a portion of the recovered and/or purified acetonitrile in a process for synthesizing an oligonudeotide and/or washing an oligonucleotide or support-bound oligonucleotide.

Advantageously, by recovering and/or purifying acetonitrile from waste acetonitrile generated during oligonucleotide synthesis processes by employing the process according to the first, second, third or fourth aspect of the present disclosure, including their embodiments disclosed herein, an oligonucleotide product, e.g., a therapeutic oligonucleotide, can be successfully synthesized without adversely affecting the quality, yield and/or purity of said product.

All the aforementioned embodiments in relation to the processes for recovering and/or purifying acetonitrile from waste acetonitrile according to the first, second, third or fourth aspects of the present disclosure are equally applicable to the processes of the disclosure for synthesizing an oligonucleotide, and may be combined.

Furthermore, all the aforementioned embodiments and embodiments hereinafter relating to the processes for recovering and/or purifying acetonitrile from waste acetonitrile of the present disclosure are equally applicable to:
acetonitrile of the present disclosure;
uses of the present disclosure; and
systems of the present disclosure.

As used herein, references to the first, second and third distillation zones of the present disclosure are for descriptive purposes only and are not intended to imply any technical limitation on the operation or position of the distillation zones within the processes or systems disclosed herein.

### Acetonitrile of the Present Disclosure

In a further aspect, provided herein is acetonitrile (or an acetonitrile composition) for use in oligonucleotide manufacture having a purity of at least 99.90%, e.g., when measured by gas chromatography, wherein the acetonitrile has been recovered from oligonucleotide synthesis waste, e.g., according to any one of the processes disclosed herein. Preferably, the acetonitrile has a purity of at least 99.90% and less than 30 parts per million water, e.g., when measured by gas chromatography. Ideally, oligonucleotide, e.g., DNA or RNA, synthesis grade acetonitrile should have a purity of at least 95% (area %), e.g., at least 99.90% (area %) acetonitrile by GC, and contain approximately 50 ppm or less of water. Oligonucleotide synthesis grade acetonitrile is typically used as a washing agent, reaction solvent, and a diluent in the oligonucleotide synthesis processes. Oligonucleotide synthesis grade acetonitrile is also used in the manufacture of therapeutic oligonucleotides. The acetonitrile of the present disclosure is preferably that obtained from the processes of the present disclosure, e.g., according to any one of the first, second, third and fourth aspects and embodiments therein. The acetonitrile of the present disclosure, e.g., obtained from the processes of the present disclosure, e.g., according to any one of the first, second, third and fourth aspects and embodiments therein, has a purity of at least 99.90%, e.g., when measured by gas chromatography. Preferably, the acetonitrile has a purity of at least 99.90% and less than 30 ppm water, e.g., when measured by gas chromatography, thus making it suitable for use/re-use in oligonucleotide manufacture.

The recovered acetonitrile may contain trace amounts of organic impurities such as pyridine, ethanol, toluene, acrylonitrile or mixtures thereof. In an embodiment, the acetonitrile comprises 1 to 10 ppm ethanol, e.g., when measured by gas chromatography. In an embodiment, the acetonitrile comprises 1 to 5 ppm pyridine, e.g., when measured by gas chromatography. In an embodiment, the acetonitrile comprises 1 to 10 ppm toluene, e.g., when measured by gas chromatography.

### Uses of the Present Disclosure

In a further aspect, there is provided the use of acetonitrile for oligonucleotide synthesis, wherein the acetonitrile has a purity of at least 99.90%, e.g., when measured by gas chromatography, and wherein the acetonitrile has been recovered from oligonucleotide synthesis waste. Preferably, the acetonitrile has a purity of at least 99.90% and less than 30 ppm water, e.g., measured by gas chromatography. Since oligonucleotide, e.g., DNA or RNA synthesis grade acetonitrile must have a purity of at least 95% (area %) when measured by GC, e.g., at least 99.90%, and contain approximately 50 ppm or less of water, such recovered acetonitrile according to the processes disclosed herein can advantageously be used or reused in oligonucleotide manufacture and subsequently recovered. The recovered acetonitrile of the present disclosure is preferably that obtained from the processes of the present disclosure, e.g., according to any one of the first, second, third and fourth aspects and embodiments therein. The acetonitrile of the present disclosure, e.g., obtained from the processes of the present disclosure, e.g., according to any one of the first, second, third and fourth aspects and embodiments therein, has a purity of at least 99.90% (area %) when measured by GC. Preferably, the acetonitrile obtained by the processes of the present disclosure has a purity of at least 99.90% (area %) and less than 30 ppm water, e.g., when measured by gas chromatography, thus making it suitable for use/re-use in oligonucleotide manufacture.

The recovered acetonitrile used in the present disclosure may contain trace amounts of organic impurities such as pyridine, ethanol, toluene, acrylonitrile or mixtures thereof. In an embodiment, the acetonitrile comprises 1 to 10 ppm ethanol, e.g., when measured by gas chromatography. In an embodiment, the acetonitrile comprises 1 to 5 ppm pyridine, e.g., when measured by gas chromatography. In an embodiment, the acetonitrile comprises 1 to 10 ppm toluene, e.g., when measured by gas chromatography. The recovered acetonitrile may comprise 1 to 10 ppm ethanol, 1 to 5 ppm pyridine and 1 to 10 ppm toluene, e.g., when measured by gas chromatography.

The disclosure thus provides a process of using the recovered acetonitrile produced according to the processes disclosed herein, in the manufacture of an oligonucleotide. In an embodiment, the oligonucleotide to be manufactured using the recovered and/or purified acetonitrile is RNA. In an embodiment, the oligonucleotide is DNA. In an embodiment, the oligonucleotide comprises both RNA and DNA.

In a further embodiment of the disclosure, the oligonucleotide to be manufactured using the recovered and/or purified acetonitrile is a modified oligonucleotide. In an embodiment, the modification is at the 2′ position of the sugar moiety and is selected from the group consisting of 2′-F, 2′-OMe, 2′-MOE, and 2′-amino, or wherein the oligonucleotide comprises a PMO, a LNA, a PNA, a BNA, or a SPIEGELMER. In an embodiment, the modification is in the nucleobase and is selected from the group consisting of a 5-methyl pyrimidine, a 7-deazaguanosine and an abasic nucleotide. In an embodiment, the modification is in the backbone and is selected from the group consisting of phosphorothioate, phosphoramidate and phosphorodiamidate.

In an embodiment, the oligonucleotide to be manufactured using the recovered and/or purified acetonitrile is an antisense oligonucleotide. In an embodiment, the oligonucleotide is an siRNA. In an embodiment, the oligonucleotide is an aptamer. In an embodiment, the oligonucleotide is an miRNA. In an embodiment, the oligonucleotide is a gapmer.

In an embodiment, the oligonucleotide to be manufactured using the recovered and/or purified acetonitrile is 10 to 200 nucleotides long. In a further embodiment of the disclosure the oligonucleotide is 15 to 30 nucleotides long. In an embodiment of the disclosure the product is 21, 22, 23, 24, 25, 26, 27, 28, 29 or 30 nucleotides long. In an embodiment of the disclosure the oligonucleotide is 20 nucleotides long, a “20-mer”. In an embodiment of the disclosure the oligonucleotide is 21 nucleotides long, a “21-mer”. In an embodiment of the disclosure the oligonucleotide is 22 nucleotides long, a “22-mer”. In an embodiment of the disclosure the oligonucleotide is 23 nucleotides long, a “23-mer”. In an embodiment of the disclosure the oligonucleotide is 24 nucleotides long, a “24-mer”. In an embodiment of the disclosure the oligonucleotide is 25 nucleotides long, a “25-mer”. In an embodiment of the disclosure the oligonucleotide is 26 nucleotides long, a “26-mer”. In an embodiment of the disclosure the oligonucleotide is 27 nucleotides long, a “27-mer”. In an embodiment of the disclosure the oligonucleotide is 28 nucleotides long, a “28-mer”. In an embodiment of the disclosure the oligonucleotide is 29 nucleotides long, a “29-mer”. In an embodiment of the disclosure the oligonucleotide is 30 nucleotides long, a “30-mer”.

In an embodiment of the disclosure, the oligonucleotide to be manufactured using the recovered and/or purified acetonitrile is a therapeutic oligonucleotide. In an embodiment of the disclosure, the oligonucleotide is a single stranded therapeutic oligonudeotide. In an embodiment of the disclosure, the oligonucleotide is a double stranded therapeutic oligonucleotide.

In an embodiment, the oligonucleotide to be manufactured using the recovered and/or purified acetonitrile is inclisiran. In another embodiment, the oligonucleotide to be manufactured using the recovered and/or purified acetonitrile is pelacarsen.

The oligonucleotide of the oligonucleotide synthesis waste from which the acetonitrile is recovered and/or purified according to the processes disclosed herein, can be the same oligonucleotide or different from the oligonucleotide to be manufactured according to the disclosure. Thus, the oligonucleotide of the oligonucleotide synthesis waste may be any oligonucleotide described above.

As mentioned herein, the acetonitrile recovered according to the processes disclosed herein is in a quantity particularly suitable for large scale and industrial manufacture of oligonucleotides, in particular the oligonucleotides disclosed herein. In the context of the present disclosure, large scale manufacture of oligonucleotides means manufacture at a scale greater than or equal to 1 litre. Alternatively or in addition, in the context of the present disclosure, large scale manufacture of oligonucleotides means manufacture at gram scale of product, in particular the production of greater than or equal to 10 grams of product. In an embodiment of the disclosure, the amount of oligonucleotide product produced is at gram scale. In an embodiment of the disclosure, the amount of product produced is greater than or equal to: 1, 2, 3, 4, 5, 6, 7, 8, 9, 10, 20, 30, 40, 50, 60, 70, 80, 90 or 100 grams. In an embodiment of the disclosure, the amount of oligonucleotide product produced is greater than or equal to: 150, 200, 250, 300, 350, 400, 450, 500, 550, 600, 650, 700, 750, 800, 850, 900, 950 grams. In an embodiment of the disclosure, the amount of oligonucleotide product produced is 500 grams or greater. In an embodiment of the disclosure, the oligonucleotide product produced using the recovered acetonitrile is at kilogram scale. In an embodiment of the disclosure, the amount of oligonucleotide product produced using the recovered acetonitrile is 1 kg or more. In an embodiment of the disclosure, the amount of oligonucleotide product produced using the recovered acetonitrile is greater than or equal to: 1, 2, 3, 4, 5, 6, 7, 8, 9, 10 kg.

In a particular embodiment, the recovered acetonitrile is used in industrial scale manufacture of an oligonucleotide.

### Systems of the Present Disclosure

In a further aspect, there is provided a system for purifying and/or recovering acetonitrile from waste acetonitrile (105, 205, 301) generated during an oligonucleotide manufacturing process, the system comprising:
a first distillation column (106, 206, 304) configured to receive an acetonitrile organic waste stream (105, 205, 301) and produce a first distillate (107, 207, 306) comprising acetonitrile and a first set of organic impurities, the first distillation column (106, 206, 304) having a condenser connected to the upper portion of the first distillation column; and
a second distillation column (112, 212, 308) configured to receive the first distillate (107, 207, 306) and separate the first set of organic impurities from the acetonitrile, and produce purified acetonitrile as the second distillation column bottoms; wherein
the system further comprises a watering zone (109, 209, 303, 307) located such that
the first distillate (107, 207, 306) is first enriched with water before reaching the second distillation column (112, 212, 308), such that purified and/or recovered acetonitrile can be obtained; or
the organic waste acetonitrile (301) is enriched with water before reaching the first distillation column (304), such that purified and/or recovered acetonitrile can be obtained.

By incorporating a watering zone into systems and processes of the present disclosure, separation of azeotrope formers with acetonitrile, such as toluene, acrylonitrile, ethanol, ethyl acetate, and low boiling amines, which are difficult to successfully separate from acetonitrile in order to produce an acceptable purity profile, can be achieved. Thus, by utilizing water as an entrainer, successful partitioning and grouping of organic waste from acetonitrile process streams can be achieved. The first set of organic impurities comprise azeotrope formers, as described herein, and impurities having a lower boiling temperature than acetonitrile.

The watering zone (109, 209, 303, 307) is configured to receive water by direct addition of water or from another source, such as an acetonitrile process stream comprising water.

The light boiling acetonitrile stream (107, 207, 306) may be fed to watering zone (109, 209, 307) whereby the acetonitrile stream comprising light boiling impurities (107, 207, 306) is enriched with water to produce a water enriched acetonitrile stream (111, 210, 210a, 307a). The water can be directly added to the first distillate (107, 207, 306) or may be added to the first distillate (107, 207, 306) as a component of a further acetonitrile process stream, e.g., present in an acetonitrile distillate from an aqueous purification step. Alternatively, the water can be added as described above to an incoming acetonitrile organic waste process stream (301), e.g., present in an acetonitrile aqueous waste stream (302) before entering the first distillation column (304).

Thus, the addition of water via an acetonitrile process stream avoids the need for the additional enrichment with water.

The water enriched acetonitrile stream (111, 201, 210a, 307a) is then fed to a second distillation column (112, 212, 308). The acetonitrile bottoms product (113, 213, 309) produced from the second distillation column may then be collected or processed further. For example, the acetonitrile bottoms product produced from distillation column (112) can be mixed with the light boiling acetonitrile stream (103) from an aqueous purification step or may be subjected to further purification steps, if necessary, to reduce the water content to below 30 ppm, as described herein.

In a further aspect, there is provided a system (100) for purifying and/or recovering acetonitrile from waste acetonitrile (101, 105) generated during an oligonucleotide manufacturing process according to the first aspect of the present disclosure, the system comprising:
a first distillation column (102) configured to receive an acetonitrile aqueous waste stream (101) and produce a first distillate (103) comprising acetonitrile and a first set of impurities, the first distillation column (102) having a condenser connected to the upper portion of the first distillation column (102), e.g., connected to an overhead stream.
a second distillation column (106) configured to receive an acetonitrile organic waste stream (105) and produce a second distillate (107) comprising acetonitrile and a first set of impurities, the second distillation column (106) having a condenser connected to the upper portion of the second distillation column (106), e.g., connected to an overhead stream; and
a third distillation column (112) configured to receive the second distillate (107) and separate the first set of organic impurities from the acetonitrile, and produce purified acetonitrile as the third distillation column bottoms; wherein
the system further comprises a watering zone (109) located such that the second distillate (107) is first enriched with water before reaching the third distillation column (112), such that purified and/or recovered acetonitrile can be obtained; and a mixing zone (116) configured to mix the first distillate (103) from the first distillation column (102) and the third distillation column bottoms (113) from the third distillation column (112), e.g., before entering a fourth distillation column (117).

The watering zone (109) may be located at any point such that the second distillate (107) is first enriched with water before reaching the third distillation column (112). For example, the watering zone (109) is located between the second and third distillation columns (106, 112).

In an embodiment, there is provided a system (100) as represented by FIG. 1.

FIG. 1 illustrates an acetonitrile purification/recycling system in one aspect of the present disclosure. In accordance with the present disclosure, an acetonitrile organic waste stream is directed into the lower portion of a first distillation column. By way of example, feedstock is low grade acetonitrile from oligonucleotide synthesis waste. The low grade acetonitrile may be provided from other sources such as, but not limited to, HPLC waste and pharmaceutical drug manufacturing waste. As previously discussed, oligonucleotide synthesis waste typically contains many constituents which can vary from one process to another. It is appreciated that the acetonitrile feedstock may contain more or fewer constituents.

The first distillation zone can be termed the organic purification zone and comprises at least two distillation columns (106, 112). The organic waste stream fed to this zone contains a combination of acetonitrile, 2,6-lutidine, acetic anhydride, dichloroacetic acid, pyridine, toluene, 1-methyl imidazole, 5-ethylthio-1H-tetrazole and lesser amounts of diethylamine, diisopropylamine, acetic acid, 5-ethylthio-1H-tetrazole, N,N-dimethylformamide, organic iodine and sulphur compounds, and residual oligonucleotide building blocks and trace amounts of water. The heavy organics produced as a bottoms product in the first distillation column are considered waste. The concentration of compounds in solution can be present at concentrations ranging from fractions of up to 30 wt %. Many of these compounds are difficult to separate from acetonitrile as they either have very similar volatilities as acetonitrile within certain concentration ranges, or they form azeotropic mixtures with acetonitrile. A number of purification stages in this first distillation zone can be employed to separate acetonitrile from the other organic impurities present. The first stage can be used to first separate a heavy boiling fraction of compounds from acetonitrile, light boilers and azeotrope formers. This stage can be designed to take advantage of even the smallest difference in relative volatility of the compounds to be separated to increase the purity of the acetonitrile stream.

Generally, distillation columns (106), (112) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

Column (106, 112) is used as a distillation column to affect separation as illustrated and hereinafter described with reference to FIG. 1. Based on the known binary azeotropic and normal pure component boiling temperatures, essentially all of the acetonitrile, acetonitrile/water azeotrope, toluene, toluene/water azeotrope, toluene/acetonitrile azeotrope, are rectified to produce column outlet vapour. Upon exiting column (106), the vapour is condensed within condenser to produce distillate (107).

The light boiling fraction (107) from this stage containing the majority of the acetonitrile can then be fractionated in a further stage, e.g., in a second distillation column (112), to produce a substantially pure acetonitrile product stream (113) and a more volatile light boiler waste stream (114). Generally, distillation column (112) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required. In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In order to separate azeotrope formers with acetonitrile, such as toluene, acrylonitrile or ethanol, an entrainer such as water can be added to the feed (107) that will produce lower boiling azeotropes relative to acetonitrile with these compounds making them easier to separate from acetonitrile. Thus, light boiling acetonitrile stream (107) is then fed to watering zone (109) followed by feeding the water enriched acetonitrile stream (111) to a further distillation column (112). The acetonitrile bottoms product (113) produced from distillation column (112) may then be fed into the third distillation zone. The acetonitrile bottoms product produced from distillation column (112) is mixed with the light boiling stream (103) from the second distillation zone.

The second distillation zone can be termed an inorganic aqueous purification zone. The acetonitrile waste entering this zone is separated into a light boiling stream (103) enriched in acetonitrile and containing some water and ethanol. The heavy boiling portion (102) contains non-volatile salts and organic residue from the main process purification as an aqueous solution. This zone is operated to maximize the yield of acetonitrile in the form of an azeotrope while removing as much excess water as possible from the process.

Generally, the column (102) will contain a structured packing or physical trays or plates, the goal of which is to provide sufficient surface are to promote intimate contact between the liquid and vapour phases moving in counter-current directions inside the column. The height of packing in the column along with the hydraulic characteristics of the gas and liquid will determine the number of separation stages contained inside the column. The number of stages required for a particular separation is determined by the complexity of the separation required.

In general, it can be said that the closer are the boiling points between the compound of interest and the other accompanying compounds, the more stages are required to effect the desired separation. The column can be operated to improve the purity of the desired compound and increase the separation of impure compounds by changing the operating pressure, the feed location and the reflux ratio.

In a further aspect, there is provided a system (200) for purifying and/or recovering a acetonitrile from waste acetonitrile (201, 205) generated during an oligonucleotide manufacturing process according to the second aspect of the present disclosure, the system comprising:
a first distillation column (202) configured to receive an acetonitrile aqueous waste stream (201) and produce a first distillate (203) comprising acetonitrile and a first set of impurities, the first distillation column having a condenser connected to the upper portion of the first distillation column (202), e.g., connected to an overhead stream;
a second distillation column (206) configured to receive an acetonitrile organic waste stream (205) and produce a second distillate (207) comprising acetonitrile and a first set of impurities, the second distillation column (206) having a condenser connected to the upper portion of the second distillation column (206), e.g., connected to an overhead stream; and
a third distillation column (212) configured to receive the first and second distillates (203, 207) and separate the first sets of impurities from the first and second distillates, and produce purified acetonitrile as the third distillation column bottoms (213); wherein
the system further comprises a watering zone (209) located such that the first and second distillates (203, 207) are first mixed and enriched with water before reaching the third distillation column (212), such that purified and/or recovered acetonitrile can be obtained.

The watering zone (209) may be located at any point such that the first and second distillates (203, 207) are first mixed and enriched with water before reaching the third distillation column (212). For example, the watering zone (209) is located between the first and third (202, 212) or the second and third distillation columns (206, 212).

In an embodiment, there is provided a system (200) as represented by FIG. 2.

FIG. 2 illustrates an acetonitrile purification/recycling system in one aspect of the present disclosure. In accordance with the present disclosure, two waste streams (201, 205), e.g., aqueous and organic waste streams, which emanate from the purification area of the oligonucleotide process and the oligonucleotide synthesis section enter into the first distillation zone. The first distillation zone can be termed the dual aqueous and organic purification zone and comprises at least two distillation columns (202, 206). The process steps A and B occur in the so-called first distillation zone (FIG. 2), in which the organic and inorganic aqueous purification steps take place. The goal is that as many heavy boilers as possible are separated from acetonitrile and any other light boilers in the feed streams. The bottoms product (204, 208) from each section is sent to waste. The details of the light boiling fractions (207, 203) are the same as those for light boiling fractions (107, 103) as disclosed above.

The details of the distillation columns (202, 206) are the same as those described above for columns (102, 106).

Upon exiting column (202, 206), the vapour is condensed within condenser to produce distillate (203, 207).

The lighter boiling fractions (203, 207) are combined in a watering zone (209) to produce an acetonitrile feed (210) which is enriched in water and introduced into the light boiler column (212) in the second distillation zone (FIG. 2). The light boiler column (212) produces an acetonitrile bottoms product (213).

The systems (100, 200) may further comprise a de-watering zone which comprises a third distillation zone.

In an embodiment of system (100), the acetonitrile streams from the previous two zones (103, 113) are combined in the feed to this zone, e.g., at mixing zone (116). Along with acetonitrile and water, the feed contains residual quantities of other organics in particular toluene, acrylonitrile and ethanol emanating from distillation zones 1 and 2.

In distillation zone 3 of system (100, 200), the separation of water from acetonitrile can be achieved using a so-called pressure swing distillation system of two distillation columns operating at two different pressures (for example 0.2 and 5 bar). This system takes advantage of the difference in azeotropic composition between the two pressures to produce a pure water waste and pure acetonitrile product stream from this zone. The impurities mentioned above form light boiling azeotropes with water and acetonitrile and an enriched stream of these impurities can be removed from the process at a separate location to the aqueous and acetonitrile product streams from the process. The mixed acetonitrile feed (116, 215) from distillation zones 1 and 2 enters the first distillation column (117, 216) of the third distillation zone. The acetonitrile stream entering this column is separated into a light boiling stream (118, 217) enriched in acetonitrile and containing acetonitrile/water azeotrope. The distillation is performed below atmospheric pressure, e.g., at 0.20 bar. The water is drawn as a bottoms product (119, 218) and is sent to aqueous waste. The light boiling acetonitrile stream (118, 217) is fed to a second distillation column (120, 219) whereby a second distillation is performed at above atmospheric pressure, e.g., 5 bar, to produce lower boiling impurities (122, 221) being drawn via the top of the second distillation column (120, 219) and highly pure acetonitrile (121, 220) being drawn from the distillation column bottoms. Lower boiling impurities (122, 221) may comprise at least one of ethanol, water, ethanol/water azeotrope and acetonitrile/water azeotrope, or mixtures thereof.

The resulting acetonitrile (121, 220) has an assay purity of at least 99.90% when measured by GC based on the total content of the acetonitrile (121, 220). For the case that the water content in the product stream is not low enough an additional stage of separation can be added that could remove the residual water as an azeotrope or it could be removed in a drying zone (124, 225), e.g., by adsorption on to a stationary phase (124, 225). The resulting acetonitrile has an assay purity of at least 99.90% and a water content below 30 ppm. The details of the distillation columns in the third distillation zone as the same as those used in the first and second zones.

In a further aspect, there is provided a system (300) for purifying and/or recovering acetonitrile from waste acetonitrile (301, 302) generated during an oligonucleotide manufacturing process according to the third aspect of the present disclosure, the system comprising:
a first distillation column (304) configured to receive a mixed acetonitrile aqueous and organic waste stream and produce a first acetonitrile distillate (306) comprising acetonitrile and a first set of impurities, the first distillation column (304) having a condenser connected to the upper portion of the first distillation column (304), e.g., connected to an overhead stream;
a second distillation column (308) configured to receive the first acetonitrile distillate (306) and produce a second acetonitrile distillate (309) comprising an acetonitrile/water azeotrope, the second distillation column (308) having a condenser connected to the upper portion of the second distillation column (308), e.g., connected to an overhead stream; and
a third distillation column (311) configured to receive the second acetonitrile distillate (309) and separate acetonitrile from the acetonitrile/water azeotrope, and produce acetonitrile as the third distillation column bottoms (312); wherein
the system further comprises a watering zone (303, 307) located such that the first acetonitrile distillate (306) is first enriched with water (307) before reaching the second distillation column (308), and/or
the organic waste acetonitrile (301) is first enriched with water by mixing an acetonitrile aqueous and organic waste stream (303) before reaching the first distillation column (304), such that purified and/or recovered acetonitrile can be obtained.

The watering zone (307) may be located at any point such that the first acetonitrile distillate (306) is first enriched with water before reaching the second distillation column (308). For example, the watering zone (307) is located between the first and second distillation columns (306, 308). Alternatively, enough water can be present to act as an entrainer from the mixing of acetonitrile aqueous and organic waste streams before entering the first distillation column (304). Thus, in this embodiment, the mixing zone (303) may also act as a watering zone.

In an embodiment, there is provided a system (300) as represented by FIG. 3.

FIG. 3 illustrates an acetonitrile purification/recycling system in one aspect of the present disclosure. In accordance with the present disclosure, two waste streams (301, 302), e.g., aqueous and organic waste streams, which emanate from the purification area of the oligonucleotide process and the oligonucleotide synthesis section enter into the mixing zone (303) and are combined therein to produce an acetonitrile waste feedstock comprising aqueous and organic impurities having lower and higher boiling points relative to acetonitrile. The details of the aqueous and organic acetonitrile waste streams are equivalent to those described for the systems representing the processes according to the first, second, third and fourth aspects of the disclosure.

The combined acetonitrile waste feedstock is then introduced into a first distillation zone which comprises a first distillation column (304). The process step B′ occurs in the so-called first distillation zone (FIG. 3), in which the initial purification step takes place. The goal is that as many heavy boilers as possible are separated from acetonitrile and any other light boilers in the feed streams. The bottoms product (305) is sent to waste. The details of the light boiling fraction (306) are described under the third aspect of the disclosure for the process section for step B′.

Upon exiting column (304), the vapour is condensed within a condenser to produce distillate (306).

The lighter boiling acetonitrile fractions (306) are sent to a watering zone (307) to produce an acetonitrile feed which is enriched in water and introduced into a second distillation column (308) in the second distillation zone (FIG. 3). The second distillation column (212) produces an acetonitrile bottoms product (309) comprising an acetonitrile/water azeotrope.

The acetonitrile bottoms product (309) of the second distillation zone is then introduced into a third distillation column (311) in the third distillation zone (FIG. 3). The third distillation column separates the acetonitrile form the acetonitrile/water azeotrope and any remaining lower boiling impurities (313) to produce the purified acetonitrile as the third distillation column bottoms product (312). Lower boiling impurities (313) may comprise at least one of ethanol, water, ethanol/water azeotrope and acetonitrile/water azeotrope, or mixtures thereof.

In distillations of system (300), the separation of impurities from acetonitrile can be achieved using a so-called pressure swing distillation system of three distillation columns operating at three different pressures (e.g., example low, mid and high, e.g., 200 mbar, 960 mbar and 5 bar).

The resultant acetonitrile (312) has an assay purity of at least 99.90% when measured by GC based on the total content of the acetonitrile. For the case that the water content in the product stream is not low enough an additional stage of separation can be added that could remove the residual water as an azeotrope or it could be removed in a drying zone (124, 225), e.g., by adsorption on to a stationary phase (124, 225). The resulting acetonitrile has an assay purity of at least 99.90% and a water content below 30 ppm.

The details of the distillation columns (304, 308, 311) of the first, second and third distillation zones of the system (300) are equivalent to those disclosed for the systems (100, 200), i.e., (102, 106, 112, 202, 206, 212).

The condensers of the distillation columns present in any one of the systems disclosed herein is typically connected to the upper portion of said distillation columns as is standard in the art. In an embodiment, one or more or all condensers of distillations columns (102, 106, 112, 117, 120, 202, 206, 212, 216, 219, 304, 308, 311) are connected via an overhead stream.

### Enumerated Embodiments

Embodiment 1. A process for recovering and/or purifying acetonitrile from waste acetonitrile (105, 205, 301) generated during oligonucleotide synthesis, the process comprising the steps:
A*) introducing an organic waste feedstock (105, 205, 301) comprising acetonitrile, a first set of organic impurities having a lower boiling temperature than acetonitrile and a second set of organic impurities having a boiling temperature greater than acetonitrile, into a distillation column (106, 206, 304) and separating the acetonitrile and first set of organic impurities from the second set of organic impurities, the acetonitrile and first set of organic impurities being drawn as a vapour from said distillation column and condensed to produce a distillate (107, 207, 306), the second set of organic impurities being produced as the second distillation column bottoms (108, 208, 305);
B*) introducing the distillate (107, 207, 306) into a watering zone (109, 209, 307) to produce a water enriched acetonitrile stream (111, 210, 210a, 307a);
C*) introducing the water enriched acetonitrile stream (111, 210, 210a, 307a) into a second distillation column (112, 212, 308) and separating the first set of organic impurities from the acetonitrile, the acetonitrile being produced as the second distillation column bottoms (113, 213, 309);
such that recovered and/or purified acetonitrile is obtained.

Embodiment 2. The process according to Embodiment 1, wherein water is added to the distillate (107, 207, 306) of step B* to produce a water enriched acetonitrile stream (111, 210, 307a).

Embodiment 3. The process according to Embodiment 1, wherein the distillate (207) of step B′ is combined with an acetonitrile stream comprising water to produce a water enriched acetonitrile stream (210a).

Embodiment 4. The process according to Embodiment 3, wherein the acetonitrile stream comprising water is obtained from a distillation of an aqueous waste feedstock generated during oligonucleotide synthesis.

Embodiment 5. The process according to Embodiment 4, wherein the distillation comprises the step of:
introducing an aqueous waste feedstock (201) comprising acetonitrile, a first set of aqueous impurities having a lower boiling temperature than acetonitrile and a second set of aqueous impurities having a boiling temperature greater than acetonitrile, into a distillation column (202) and separating the acetonitrile and first set of impurities from the second set of impurities, the acetonitrile and first set of impurities being drawn as a vapour from said distillation column and condensed to produce a distillate (203), the second set of impurities being produced as the distillation column bottoms (204).

Embodiment 6. The process according to Embodiment 1, wherein the distillate (207) of step B′ is combined with an acetonitrile stream comprising water according to any one of Embodiments 3 to 5, and wherein water is further added in step B′ to produce a water enriched acetonitrile stream (210).

Embodiment 7. The process according to any one of the preceding Embodiments, wherein the distillation column of step A′ operates in the range of 50 to 980 mbar, e.g., 100 to 300 mbar.

Embodiment 8. The process according to any one of the preceding Embodiments, wherein the distillation column of step C* operates in the range of 100 to 980 mbar, e.g., 900 to 980 mbar.

Embodiment 9. The process according to any one of the preceding Embodiments, herein step C* further comprises mixing distillate (107, 207) with a recycled acetonitrile stream (122, 221) comprising an acetonitrile/water azeotrope and ethanol, wherein the recycled acetonitrile stream (122, 221) is a by-product of a further processing step, and wherein the recycled acetonitrile stream (122, 221) comprises acetonitrile, water and ethanol.

Embodiment 10. The process according to any one of the preceding Embodiments, wherein the distillation column bottoms (113, 213) is further processed to reduce the water content of the acetonitrile to less than 30 parts per million.

Embodiment 11. A process for recovering and/or purifying acetonitrile from waste acetonitrile (101, 105), e.g., generated during oligonucleotide synthesis, e.g., solid phase oligonucleotide synthesis, the process comprising the steps:
A1) introducing an aqueous waste feedstock (101) comprising acetonitrile, a first set of aqueous impurities having a lower boiling temperature than acetonitrile and a second set of aqueous impurities having a boiling temperature greater than acetonitrile, into a first distillation column (102) and separating the acetonitrile and first set of impurities from the second set of impurities, the acetonitrile and first set of impurities being drawn as a vapour from said first distillation column and condensed to produce a first distillate (103), the second set of impurities being produced as the first distillation column bottoms (104);
B1) introducing an organic waste feedstock (105) comprising acetonitrile, a first set of organic impurities having a lower boiling temperature than acetonitrile and a second set of organic impurities having a boiling temperature greater than acetonitrile, into a second distillation column (106) and separating the acetonitrile and first set of organic impurities from the second set of organic impurities, the acetonitrile and first set of organic impurities being drawn as a vapour from said second distillation column and condensed to produce a second distillate (107), the second set of organic impurities being produced as the second distillation column bottoms (108);
C1) introducing the second distillate (107) into a watering zone (109) and enriching with water to produce a water enriched acetonitrile stream (111);
D1) introducing the water enriched acetonitrile stream (111) into a third distillation column (112) and separating the first set of organic impurities from the acetonitrile, the acetonitrile being produced as the third distillation column bottoms (113); and
E1) feeding the first distillate (103) of step A1 and the third distillation column bottoms (113) of step D1 to a mixing zone (116) and combining therein, to produce an acetonitrile enriched stream (110);
such that recovered and/or purified acetonitrile is obtained.

Embodiment 12. The process according to Embodiment 11, wherein steps A1 and B1 are performed in parallel.

Embodiment 13. The process according to any one of Embodiments 11 and 12, wherein steps A1 and B1 are performed sequentially.

Embodiment 14. The process according to any one of Embodiments 11 to 13, wherein the first set of aqueous impurities of step A1 comprise at least one of ethanol and water, or mixtures thereof.

Embodiment 15. The process according to any one of Embodiments 11 to 14, wherein the distillation column of step B1 operates in the range of 50 to 980 mbar, e.g., 100 to 300 mbar.

Embodiment 16. The process according to any one of Embodiments 11 to 15, wherein the distillation column of step A1 operates in the range of 200 to 980 mbar, e.g., 400 to 500 mbar.

Embodiment 17. The process according to any one of Embodiments 11 to 16, wherein the distillation column of step D1 operates in the range of 100 to 980 mbar, e.g., 900 to 980 mbar.

Embodiment 18. The process according to any one of Embodiments 11 to 17, wherein step C1 further comprises mixing distillate (107) with a recycled acetonitrile stream (221) comprising an acetonitrile/water azeotrope and ethanol, wherein the recycled acetonitrile stream (221) is a by-product of a further processing step.

Embodiment 19. The process according to any one of Embodiments 11 to 18, wherein the reflux ratio in step B1 is between 2 to 8, e.g., 5.

Embodiment 20. The process according to any one of Embodiments 11 to 19, wherein the third distillation column bottoms (113) of step D1 comprises acetonitrile and an acetonitrile/water azeotrope.

Embodiment 21. The process according to any one of Embodiments 11 to 20, wherein the reflux ratio in step D1 is between 5 to 40, e.g., 30.

Embodiment 22. The process according to any one of Embodiments 11 to 21, wherein the first distillate (103) of step A1 comprises an acetonitrile/water azeotrope.

Embodiment 23. The process according to any one of Embodiments 11 to 22, wherein the reflux ratio in step A1 is between 0.25 to 2, e.g., 1.

Embodiment 24. The process according to any one of Embodiments 11 to 23, wherein the first distillate (103) of step A1 comprises 60 to 80 wt. % acetonitrile.

Embodiment 25. The process according to any one of Embodiments 11 to 24, wherein the first distillate (103) of step A1 20 to 40 wt. % water.

Embodiment 26. The process according to any one of Embodiments 11 to 25, wherein the water enriched acetonitrile stream (111) of step C1 comprises 5 to 40 wt. % acetonitrile.

Embodiment 27. The process according to any one of Embodiments 11 to 26, wherein the amount of water added in step C1 is in a ratio of 0.05 to 0.40 (w/w) or greater based on the total amount of acetonitrile and impurities present in the stream.

Embodiment 28. The process according to any one of Embodiments 11 to 27, wherein the third distillation column bottoms (113) of step D1 comprises 60 to 90 wt. % acetonitrile.

Embodiment 29. The process according to any one of Embodiments 11 to 28, wherein the third distillation column bottoms (113) of step D1 additionally comprises ethanol as an impurity.

Embodiment 30. The process according to Embodiment 29, wherein the concentration of ethanol as an impurity is 1 to 10 parts per million.

Embodiment 31. The process according to any one of Embodiments 11 to 30, wherein the acetonitrile enriched stream (110) of step E1 is further processed to reduce the water content of the acetonitrile to less than 30 parts per million.

Embodiment 32. A process for recovering and/or purifying acetonitrile from waste acetonitrile (201, 205), e.g., generated during oligonucleotide synthesis, e.g., solid phase oligonucleotide synthesis, the process comprising the steps:
A) introducing an aqueous waste feedstock (201) comprising acetonitrile, a first set of aqueous impurities having a lower boiling temperature than acetonitrile and a second set of aqueous impurities having a boiling temperature greater than acetonitrile, into a first distillation column (202) and separating the acetonitrile and first set of aqueous impurities from the second set of aqueous impurities, the acetonitrile and first set of aqueous impurities being drawn as a vapour from said first distillation column and condensed to produce a first distillate (203), the second set of aqueous impurities being produced as the first distillation column bottoms (204);
B) introducing an organic waste feedstock (205) comprising acetonitrile, a first set of organic impurities having a lower boiling temperature than acetonitrile and a second set of organic impurities having a boiling temperature greater than acetonitrile, into a second distillation column (206) and separating the acetonitrile and first set of organic impurities from the second set of organic impurities, the acetonitrile and first set of organic impurities being drawn as a vapour from said second distillation column and condensed to produce a second distillate (207), the second set of organic impurities being produced as the second distillation column bottoms (208);
C) feeding the first distillate of step A (203) and the second distillate of step B (207) to a watering zone (209) and
C1) combining therein to produce a water enriched acetonitrile stream (210a), or
C2) combining therein and enriching with water to produce a water enriched acetonitrile stream (210); and
D) introducing the water enriched acetonitrile stream (210, 201a) into a third distillation column (212) and separating the first set of organic and aqueous impurities from the acetonitrile, the acetonitrile being produced as the third distillation column bottoms (213);
such that recovered and/or purified acetonitrile is obtained.

Embodiment 33. The process according to Embodiment 32, wherein steps A and B are performed in parallel.

Embodiment 34. The process according to Embodiment 32, wherein steps A and B are performed sequentially.

Embodiment 35. The process according to any one of Embodiments 32 to 34, wherein the distillation column of step A operates in the range of 200 to 980 mbar, e.g., 400 to 500 mbar.

Embodiment 36. The process according to any one of Embodiments 32 to 35, wherein the distillation column of step B operates in the range of 50 to 980 mbar, e.g., 100 to 300 mbar.

Embodiment 37. The process according to any one of Embodiments 32 to 36, wherein the distillation column of step D operates in the range of 100 to 980 mbar, e.g., 900 to 980 mbar.

Embodiment 38. The process according to any one of Embodiments 32 to 37, wherein step C further comprises mixing first and second distillates (203, 207) with a recycled acetonitrile stream (221) comprising an acetonitrile/water azeotrope and ethanol, and wherein the recycled acetonitrile stream (221) is a by-product of a further processing step.

Embodiment 39. The process according to any one of Embodiments 32 to 38, wherein the first set of aqueous impurities of step A comprise at least one of ethanol and water, or mixtures thereof.

Embodiment 40. The process according to any one of Embodiments 32 to 39, wherein the first distillate (203) of step A comprises an acetonitrile/water azeotrope.

Embodiment 41. The process according to any one of Embodiments 32 to 40, wherein the reflux ratio in Step A is between 0.25 to 2, e.g., 1.

Embodiment 42. The process according to any one of Embodiments 32 to 41, wherein the reflux ratio in Step B is between 2 to 8, e.g., 5.

Embodiment 43. The process according to any one of Embodiments 32 to 42, wherein the third distillation column bottoms (213) of step D comprises acetonitrile and an acetonitrile/water azeotrope.

Embodiment 44. The process according to any one of Embodiments 32 to 43, wherein the reflux ratio in Step D is between 5 to 40, e.g., 30.

Embodiment 45. The process according to any one of Embodiments 32 to 44, wherein the first distillate (203) of step A comprises 60 to 80 wt. % acetonitrile.

Embodiment 46. The process according to any one of Embodiments 32 to 45, wherein the first distillate (203) of step A comprises 20 to 40 wt. % water.

Embodiment 47. The process according to any one of Embodiments 32 to 46, wherein the amount of water added in step C is in a ratio of 0.05 to 0.40 (w/w) or greater based on the total amount of acetonitrile and impurities present in the stream.

Embodiment 48. The process according to any one of Embodiments 32 to 47, wherein the water enriched acetonitrile stream (210, 210a) of step C comprises 60 to 90 wt. % acetonitrile.

Embodiment 49. The process according to any one of Embodiments 32 to 48, wherein the third distillation column bottoms (213) of step D comprises 60 to 90 wt. % acetonitrile.

Embodiment 50. The process according to any one of Embodiments 32 to 49, wherein the third distillation column bottoms (213) of step D additionally comprises ethanol as an impurity.

Embodiment 51. The process according to Embodiment 50, wherein the concentration of ethanol as an impurity is 1 to 10 parts per million.

Embodiment 52. The process according to any one of the preceding Embodiments, wherein the acetonitrile enriched stream (110) or the distillation column bottoms (113, 213) of is further processed to reduce the water content of the acetonitrile to less than 30 parts per million.

Embodiment 53. The process according to Embodiment 52, when dependent on any one of Embodiments 11 to 52, further comprising the step of introducing the acetonitrile enriched stream (110) or the distillation column bottoms (113, 213) into a fourth distillation column (117, 216), wherein the water content of the acetonitrile enriched stream is reduced by use of a pressure-swing distillation to recover acetonitrile having a water content of less than 30 parts per million.

Embodiment 54. The process according to Embodiment 53, wherein the pressure-swing distillation comprises the steps:
F1) introducing the acetonitrile enriched stream (110) or the distillation column bottoms (113, 213) into a distillation column (117, 216) and, performing a distillation at below atmospheric pressure to remove water therefrom, the acetonitrile being drawn as a vapour from said distillation column and condensed to produce a distillate (118, 217), and water being produced as the distillation column bottoms (119, 218);
F2) introducing the distillate (118, 217) into a distillation column (120, 219) and, performing a second distillation at above atmospheric pressure, e.g., 5 bar, to produce lower boiling fraction (122, 221) being drawn via the upper portion of the distillation column (120, 219) and acetonitrile being produced as the distillation column bottoms (121, 220).

Embodiment 55. The process according to Embodiment 54, wherein the reflux ratio in Step F1 is between 0.05 to 1, e.g., 0.1.

Embodiment 56. The process according to any one of Embodiments 54 and 55, wherein the reflux ratio in Step F2 is between 0.05 to 1, e.g., 0.25.

Embodiment 57. The process according to any one of Embodiments 54 to 56, wherein the distillate (118, 217) comprises acetonitrile and an acetonitrile/water azeotrope.

Embodiment 58. The process according to any one of Embodiments 54 to 57, wherein the lower boiling fraction of step F2 comprises at least one of ethanol, water, ethanol/water azeotrope and acetonitrile/water azeotrope, or mixtures thereof.

Embodiment 59. The process according to any one of Embodiments 54 to 58, wherein the acetonitrile produced as the distillation column bottoms (121, 220) of step F2 is passed over a water reducing adsorbent (124, 225).

Embodiment 60. The process according to Embodiment 59, wherein the water reducing adsorbent (124, 225) is molecular sieves.

Embodiment 61. The process according to any one of the preceding Embodiments, wherein the recovered acetonitrile has a purity of at least 99.90% when measured by gas chromatography and optionally, a water content of less than 50 parts per million, e.g., less than 30 parts per million.

Embodiment 62. The process according to any one of Embodiments 52 to 61, further comprising recycling at least a portion of said lower boiling fraction (122, 221) from the further processing of acetonitrile enriched stream (110) or the distillation column bottoms (113, 213).

Embodiment 63. The process according to any one of Embodiments 52 to 62, further comprising recycling at least a portion of said lower boiling fraction (122, 221) from the distillation column (120, 219) of step F2.

Embodiment 64. The process according to Embodiment 63, wherein said lower boiling fraction (122, 221) is fed back into step F1.

Embodiment 65. The process according to any one of Embodiments 63 and 64, wherein said lower boiling fraction (122, 221) is first purged (123, 222) before being recycled.

Embodiment 66. The process according to any one of Embodiments 63 to 65, wherein said lower boiling fraction (122) is recycled to step D1 via said watering zone in step C1.

Embodiment 67. The process according to Embodiment 66, wherein at least a portion of said lower boiling fraction (122, 221) is fed back into the distillation column (117, 216) of step F1.

Embodiment 68. The process according to any one of Embodiments 54 to 67, wherein said lower boiling fraction (122, 221) is fed back into the distillation column (117, 216) of step F1 via the mixing zone (215) of step E or mixing zone (116) of step E1.

Embodiment 69. The process according to Embodiment 68, wherein at least a portion of said lower boiling fraction (122, 221) is recycled back to the distillation of step D1 or step D with a greater proportion being sent back to mixing zone (116, 215).

Embodiment 70. The process according to Embodiment 69, wherein at least a portion of said lower boiling fraction (221) is recycled back to the watering zone (209) of step C.

Embodiment 71. The process according to any one of Embodiments 54 to 61, wherein the lower boiling fraction (122, 221) of step F2 is directed to waste.

Embodiment 72. The process according to Embodiment 71, wherein the lower boiling fraction (122, 221) is first purged (123, 222) before being directed to waste.

Embodiment 73. The process according to any one of Embodiments 54 to 72, wherein the distillation of step F1 is performed in the range of 50 to 980 mbar, e.g., 50 to 200 mbar, e.g., 200 mbar.

Embodiment 74. The process according to any one of Embodiments 54 to 73, wherein the distillation of step F2 is performed in the range of 3 to 8 bar, e.g., 5 bar.

Embodiment 75. A process for recovering and/or purifying acetonitrile from waste acetonitrile (301, 302) generated during oligonucleotide synthesis, the process comprising the steps:
A′) introducing an organic waste feedstock (301) comprising acetonitrile, a first set of organic impurities having a lower boiling temperature than acetonitrile and a second set of organic impurities having a boiling temperature greater than acetonitrile, and an aqueous waste feedstock (302) comprising acetonitrile a first set of aqueous impurities having a lower boiling temperature than acetonitrile and a second set of aqueous impurities having a boiling temperature greater than acetonitrile, into a first mixing zone (303), and combining therein, to form an acetonitrile waste feedstock comprising said impurities; or
introducing an organic waste feedstock (301) comprising acetonitrile, a first set of organic impurities having a lower boiling temperature than acetonitrile and a second set of organic impurities having a boiling temperature greater than acetonitrile, into a first mixing zone (303), and enriching with water, to produce a water enriched acetonitrile stream comprising said impurities;
B′) introducing the acetonitrile waste feedstock or water enriched acetonitrile stream into a first distillation column (304) and separating the acetonitrile and the first set of impurities having a lower boiling temperature than acetonitrile from the second set of impurities having a boiling temperature greater than acetonitrile, the acetonitrile and first set of impurities being drawn as a vapour from said first distillation column and condensed to produce a first distillate (306), the second set of impurities being produced as the first distillation column bottoms (305);
C′) optionally introducing the first distillate (306) comprising acetonitrile and low boiling impurities, into a watering zone (307) and enriching with water to produce a water enriched acetonitrile stream (307a); and
D′) introducing the water enriched acetonitrile stream (306, 307a) into a second distillation column (308) and separating the acetonitrile from the low boiling impurities, the acetonitrile being produced as the second distillation column bottoms (309);
   such that recovered and/or purified acetonitrile is obtained.

Embodiment 76. The process according to Embodiment 75, wherein the second distillation column bottoms comprises an acetonitrile/water azeotrope.

Embodiment 77. The process according to Embodiment 76, further comprising the step of
E′) introducing the second distillation column bottoms product (309) of step D′ into a third distillation column (311) and separating the acetonitrile from the acetonitrile/water azeotrope, the acetonitrile being produced as the third distillation column bottoms (312).

Embodiment 78. The process according to any one of Embodiments 75 to 77, wherein step A′ further comprises mixing the acetonitrile waste feedstock with a recycled acetonitrile stream (316) comprising an acetonitrile/water azeotrope and ethanol, wherein the recycled acetonitrile stream (316) is a by-product of a further processing step.

Embodiment 79. The process according to any one of Embodiments 75 to 78, wherein the second distillation column bottoms (309) or the third distillation column bottoms (312) is further processed to reduce the water content of the acetonitrile to less than 30 parts per million.

Embodiment 80. The process according to any one of Embodiments 75 to 79, wherein the distillation of step B′ is performed at a lower pressure than the distillation of step D′, and optionally the distillation of step D′ is performed at a lower pressure than step E′.

Embodiment 81. The process according to any one of Embodiments 75 to 80, wherein the distillation column of step B′ operates in the range of 50 to 980 mbar, e.g., 100 to 300 mbar.

Embodiment 82. The process according to any one of Embodiments 75 to 81, wherein the distillation column of step D′ operates in the range of 50 to 980 mbar, e.g., 100 to 300 mbar.

Embodiment 83. The process according to any one of Embodiments 77 to 82, wherein the distillation column of step E′ operates in the range of 50 to 980 mbar, e.g., 100 to 300 mbar.

Embodiment 84. The process according to any one of Embodiments 77 to 83, wherein the recovered acetonitrile (312) is passed over a water reducing adsorbent.

Embodiment 85. The process according to Embodiment 84, wherein the water reducing adsorbent is molecular sieves.

Embodiment 86. The process according to any one of Embodiments 75 to 85, wherein the first distillate (306) of step B′ comprises an acetonitrile/water azeotrope.

Embodiment 87. The process according to any one of Embodiments 75 to 86, wherein the reflux ratio in step B′ is between 0.25 to 8, e.g., 0.25 to 2 or 2 to 8 and/or the reflux ratio of step D′ is between 5 to 40, e.g., 30.

Embodiment 88. The process according to any one of Embodiments 75 to 87, wherein the second distillation column bottoms (309) of step D′ comprises acetonitrile and an acetonitrile/water azeotrope.

Embodiment 89. The process according to any one of Embodiments 75 to 88, wherein the amount of water added in step C′ is in a ratio of 0.05 to 0.40 (w/w) or greater based on the total amount of acetonitrile and impurities present in the stream.

Embodiment 90. The process according to any one of Embodiments 11 to 89, wherein the acetonitrile and first set of aqueous impurities are drawn from the upper portion, e.g., from the top of the distillation column (102, 202, 304) in step A1, step A or step B′.

Embodiment 91. The process according to any one of the preceding Embodiments, wherein the first set of organic impurities of step A*, step B1, step B or step B′ comprises azeotrope formers, which azeotrope formers comprise at least one of toluene, ethyl acetate, ethanol, diethylamine, diisopropylamine and acrylonitrile, or mixtures thereof.

Embodiment 92. The process according to any one of the preceding Embodiments, wherein the first set of organic impurities of step A*, step B1, step B or step B′ comprises at least one of diethylamine, diisopropylamine, ethyl acetate and acrylonitrile, or mixtures thereof.

Embodiment 93. The process according to any one of the preceding Embodiments, wherein the second set of organic impurities of step A*, step B1, step B or step B′ comprises at least one of toluene, acetic acid, acetic anhydride, dichloroacetic acid and N-containing compound, or mixtures thereof.

Embodiment 94. The process according to Embodiment 93, wherein the N-containing compound is selected from imidazole, 1-methylimidazole, 2,6-lutidine, dimethylformamide, pyridine, acrylonitrile, diisopropylamine, diethylamine, phenyl acetyl(disulfide), and 5-(ethylthio)-1H-tetrazole.

Embodiment 95. The process according to any one of the preceding Embodiments, wherein the water enriched acetonitrile stream (111, 210, 210a, 303a, 307a) comprises acetonitrile and lower boiling azeotropes relative to acetonitrile.

Embodiment 96. The process according to Embodiment 95, wherein the lower boiling azeotropes comprise at least one of acetonitrile, water, toluene, ethanol, diethylamine, diisopropylamine, and acrylonitrile, or mixtures thereof.

Embodiment 97. The process according to any one of Embodiments 95 and 96, wherein the lower boiling azeotropes comprise at least one of acetonitrile/water, acetonitrile/toluene, acetonitrile/diethylamine, acetonitrile/diisopropylamine, toluene/water, ethanol/water, toluene/water/acrylonitrile, toluene/water/acetonitrle, and diisopropylamine/water/acetonitrile, or mixtures thereof.

Embodiment 98. The process according to any one of Embodiments 77 to 97, further comprising recycling at least a portion of said lower boiling fraction (313) from the third distillation column (311).

Embodiment 99. The process according to Embodiment 98, wherein the at least a portion of said lower boiling fraction (313) is fed back into the mixing zone (303).

Embodiment 100. The process according to Embodiment 99, wherein at least a portion of said lower boiling fraction (313) is first purged (314) before being fed back into the mixing zone (303).

Embodiment 101. The process according to any one of Embodiments 77 to 97, wherein the lower boiling fraction (313) is purged (314) and sent to waste (315).

Embodiment 102. The process according to any one of the preceding Embodiments, wherein the recovered acetonitrile has a purity of at least 99.90% (area %) when measured by gas chromatography.

Embodiment 103. The process according to any one of the preceding Embodiments, wherein the waste acetonitrile is of industrial scale.

Embodiment 104. The process according to any one of the preceding Embodiments, wherein the waste acetonitrile is at least 10 kg, preferably at least 100 kg, at least 1000 kg or at least 10,000 kg.

Embodiment 105. The process according to any one of the preceding Embodiments, wherein the acetonitrile is recovered at the gram or kilogram scale, or greater.

Embodiment 106. The process according to any one of the preceding Embodiments, wherein the process is carried out in batch.

Embodiment 107. The process according to any one of Embodiments 1 to 105, wherein the process is carried out on a continuous basis.

Embodiment 108. The process according to any one of the preceding Embodiments, wherein the oligonucleotide synthesis is solid phase oligonucleotide synthesis.

Embodiment 109. The process according to any one of the preceding Embodiments, wherein the oligonucleotide is RNA.

Embodiment 110. The process according to any one of Embodiments 1 to 108, wherein the oligonucleotide is DNA.

Embodiment 111. The process according to any one of Embodiments 1 to 108, wherein the oligonucleotide comprises both RNA and DNA.

Embodiment 112. The process according to any one of the preceding Embodiments, wherein the oligonucleotide is a modified oligonucleotide.

Embodiment 113. The process according to Embodiment 112, wherein the modification is at the 2′ position of the sugar moiety and is selected from the group consisting of 2′-F, 2′-OMe, 2′-MOE, and 2′-amino, or wherein the oligonucleotide comprises a PMO, a LNA, a PNA, a BNA, or a SPIEGELMER.

Embodiment 114. The process according to any one of Embodiments 112 and 113, wherein the modification is in the nucleobase and is selected from the group consisting of a 5-methyl pyrimidine, a 7-deazaguanosine and an abasic nucleotide.

Embodiment 115. The process according to any one of Embodiments 112 to 114, wherein the modification is in the backbone and is selected from the group consisting of phosphorothioate, phosphoramidate and phosphorodiamidate.

Embodiment 116. The process according to any one of Embodiments 112 to 115, wherein the oligonucleotide is selected from an antisense oligonucleotide, an aptamer, siRNA, miRNA, and a gapmer.

Embodiment 117. The process according to any one of Embodiments 112 to 116, wherein the oligonucleotide is an antisense oligonucleotide.

Embodiment 118. The process according to any one of Embodiments 112 to 116, wherein the oligonucleotide is an siRNA.

Embodiment 119. The process according to any one of the preceding Embodiments, wherein the oligonucleotide is 10 to 200 nucleotides long.

Embodiment 120. The process according to any one of the preceding Embodiments, wherein the oligonucleotide is 18, 19, 20, 21, 22, 23, 24, 25, 26, 27, 28, 29 or 30 nucleotides long.

Embodiment 121. The process according to any one of the preceding Embodiments, wherein the oligonudeotide is an 18-mer, 19-mer, 20-mer, 21-mer, 22-mer, 23-mer, 24-mer, 25-mer, 26-mer, 27-mer, 28-mer, 29-mer or a 30-mer.

Embodiment 122. The process according to any one of the preceding Embodiments, wherein the oligonucleotide is a therapeutic oligonucleotide.

Embodiment 123. The process according to any one of the preceding Embodiments, wherein the oligonucleotide is a single stranded therapeutic oligonucleotide.

Embodiment 124. The process according to any one of the preceding Embodiments, wherein the oligonucleotide is a double stranded therapeutic oligonucleotide.

Embodiment 125. The process according to any one of Embodiments 1 to 109, 112 to 117, 119 to 122 and 124, wherein the oligonucleotide is inclisiran.

Embodiment 126. The process according to any one of Embodiments 1 to 109, 112 to 116 and 118 to 123, wherein the oligonucleotide is pelacarsen.

Embodiment 127. Acetonitrile (or an acetonitrile composition) for use in oligonucleotide manufacture having a purity of at least 99.90%, when measured by gas chromatography, wherein the acetonitrile has been recovered from oligonucleotide synthesis waste.

Embodiment 128. Acetonitrile according to Embodiment 127, wherein the acetonitrile has less than 30 parts per million water.

Embodiment 129. Acetonitrile according to any one of Embodiments 127 and 128, wherein the acetonitrile additionally comprises 1 to 5 parts per million pyridine, e.g., when measured by gas chromatography.

Embodiment 130. Acetonitrile according to any one of Embodiments 127 to 129, wherein the acetonitrile additionally comprises 1 to 10 parts per million ethanol, e.g., when measured by gas chromatography.

Embodiment 131. Acetonitrile according to any one of Embodiments 127 to 130, wherein the acetonitrile additionally comprises 1 to 10 parts per million toluene, e.g., when measured by gas chromatography.

Embodiment 132. Acetonitrile obtained by the process according to any one of Embodiments 1 to 126.

Embodiment 133. Acetonitrile according to Embodiment 132, wherein the acetonitrile has a purity of at least 99.90%.

Embodiment 134. Acetonitrile according to any one of Embodiments 132 and 133, wherein the acetonitrile has a purity of at least 99.90% and less than 30 parts per million water, when measured by gas chromatography.

Embodiment 135. Acetonitrile according to any one of Embodiments 132 to 134, wherein the acetonitrile additionally comprises 1 to 5 parts per million pyridine, e.g., when measured by gas chromatography.

Embodiment 136. Acetonitrile according to any one of Embodiments 132 to 135, wherein the acetonitrile additionally comprises 1 to 10 parts per million toluene, e.g., when measured by gas chromatography.

Embodiment 137. Acetonitrile according to any one of Embodiments 132 to 136, wherein the acetonitrile additionally comprises 1 to 10 parts per million ethanol, e.g., when measured by gas chromatography.

Embodiment 138. A process for synthesizing an oligonucleotide, the process comprising recovering and/or purifying acetonitrile from waste acetonitrile according to any one of Embodiments 1 to 126, and

using at least a portion of the recovered and/or purified acetonitrile in a process for synthesizing an oligonucleotide and/or washing an oligonucleotide or support-bound oligonucleotide.

Embodiment 139. The process according to Embodiment 138, wherein the process comprises solid phase oligonucleotide synthesis.

Embodiment 140. The process according to any one of claims 138 and 139, wherein the oligonucleotide to be synthesized is defined according to any one of Embodiments 109 to 126.

Embodiment 141. A system for purifying and/or recovering acetonitrile from waste acetonitrile (105, 205, 301) generated during an oligonucleotide manufacturing process, the system comprising:
a first distillation column (106, 206, 304) configured to receive an acetonitrile organic waste stream (105, 205, 301) and produce a first distillate (107, 207, 306) comprising acetonitrile and a first set of impurities, the first distillation column (106, 206, 304) having a condenser connected to the upper portion of the first distillation column; and
a second distillation column (112, 212, 308) configured to receive the first distillate (107, 207, 306) and separate the first set of organic impurities from the acetonitrile, and produce purified acetonitrile as the second distillation column bottoms; wherein
the system further comprises a watering zone (109, 209, 303, 307) located such that
the first distillate (107, 207, 306) is first enriched with water before reaching the second distillation column (112, 212, 308), such that purified and/or recovered acetonitrile can be obtained; or
the waste acetonitrile (301) is enriched with water before reaching the first distillation column (304), such that purified and/or recovered acetonitrile can be obtained.

Embodiment 142. A system (100) for purifying and/or recovering acetonitrile from waste acetonitrile (101, 105) generated during an oligonucleotide manufacturing process, e.g., solid phase oligonucleotide manufacturing process, the system comprising:
a first distillation column (102) configured to receive an acetonitrile aqueous waste stream (101) and produce a first distillate (103) comprising acetonitrile and a first set of impurities, the first distillation column (102) having a condenser connected to the upper portion of the first distillation column (102), e.g., connected to an overhead stream;
a second distillation column (106) configured to receive an acetonitrile organic waste stream (105) and produce a second distillate (107) comprising acetonitrile and a first set of impurities, the second distillation column (106) having a condenser connected to the upper portion of the second distillation column (106), e.g., connected to an overhead stream; and
a third distillation column (112) configured to receive the second distillate (107) and separate the first set of organic impurities from the acetonitrile, and produce purified acetonitrile as the third distillation column bottoms; wherein
the system further comprises a watering zone (109) located such that the second distillate (107) is first enriched with water before reaching the third distillation column (112), such that purified and/or recovered acetonitrile can be obtained; and a mixing zone (116) configured to mix the first distillate (103) from the first distillation column (102) and the third distillation column bottoms (113) from the third distillation column (112).

Embodiment 143. A system (200) for purifying and/or recovering a acetonitrile from waste acetonitrile (201, 205) generated during an oligonucleotide manufacturing process, e.g., solid phase oligonucleotide manufacturing process, the system comprising:
a first distillation column (202) configured to receive an acetonitrile aqueous waste stream (201) and produce a first distillate (203) comprising acetonitrile and a first set of impurities, the first distillation column having a condenser connected to the upper portion of the first distillation column (202), e.g., connected to an overhead stream;
a second distillation column (206) configured to receive an acetonitrile organic waste stream (205) and produce a second distillate (207) comprising acetonitrile and a first set of impurities, the second distillation column (206) having a condenser connected to the upper portion of the second distillation column (206), e.g., connected to an overhead stream; and
a third distillation column (212) configured to receive the first and second distillates (203, 207) and separate the first sets of impurities from the first and second distillates, and produce purified acetonitrile as the third distillation column bottoms (213); wherein
the system further comprises a watering zone (209) located such that the first and second distillates (203, 207) are first mixed or are first mixed and enriched with water before reaching the third distillation column (212), such that purified and/or recovered acetonitrile can be obtained.

Embodiment 144. A system (300) for purifying and/or recovering acetonitrile from waste acetonitrile (301, 302) generated during an oligonucleotide manufacturing process, the system comprising:
a first distillation column (304) configured to receive a mixed acetonitrile aqueous and organic waste stream and produce a first acetonitrile distillate (306) comprising acetonitrile and a first set of impurities, the first distillation column (304) having a condenser connected to the upper portion of the first distillation column (304), e.g., connected to an overhead stream;
a second distillation column (308) configured to receive the first acetonitile distillate (306) and produce a second acetonitrile distillate (309) comprising an acetonitrile/water azeotrope, the second distillation column (308) having a condenser connected to the upper portion of the second distillation column (308), e.g., connected to an overhead stream; and
a third distillation column (311) configured to receive the second acetonitrile distillate (309) and separate acetonitrile from the acetonitrile/water azeotrope, and produce acetonitrile as the third distillation column bottoms (312); wherein
the system further comprises a watering zone (303, 307) located such that the first acetonitrile distillate (306) is enriched with water (307) before reaching the second distillation column (308), and/or
the organic waste acetonitrile (301) is enriched with water by mixing an acetonitrile aqueous and organic waste stream (303) before reaching the first distillation column (304), such that purified and/or recovered acetonitrile can be obtained.

Embodiment 145. The system (100) according to Embodiment 142, as shown in FIG. 1.

Embodiment 146. The system (200) according to Embodiment 143, as shown in FIG. 2.

Embodiment 147. The system (300) according to Embodiment 144, as shown in FIG. 3.

Embodiment 148. The system (100, 200) according to any one of Embodiments 142 and 143, further comprising at least a fourth distillation column (117, 216) configured to receive the third distillation column bottoms (113, 213) of the third distillation column (112, 212) and produce a fourth distillate (118, 217) comprising acetonitrile.

Embodiment 149. The system (100, 200) according to Embodiment 148, further comprising fifth distillation column (120, 219) configured to receive the fourth distillate (118, 217) from the fourth distillation column (117, 216), and produce a fifth distillation column bottoms (121, 220) product comprising highly pure acetonitrile.

Embodiment 150. The system (100, 200, 300) according to any one of Embodiments 141 to 149, further comprising a drying zone (124, 225), wherein the drying zone is configured to reduce the water content in the acetonitrile product to less than 30 parts per million.

Embodiment 151. The system (100, 200, 300) according to any one of Embodiments 141 to 150, wherein the purified and/or recovered acetonitrile has a purity of at least 99.90% when measured by gas chromatography.

Embodiment 152. Use of acetonitrile for oligonucleotide synthesis, wherein the acetonitrile has a purity of at least 99.90% when measured by gas chromatography, and wherein the acetonitrile has been recovered from oligonucleotide synthesis waste, optionally wherein the oligonucleotide synthesis waste is solid phase oligonucleotide synthesis waste.

Embodiment 153. The use according to Embodiment 152, wherein the acetonitrile has less than 30 parts per million water.

Embodiment 154. The use according to any one of Embodiments 152 and 153, wherein the acetonitrile is defined according to any one of Embodiments 127 to 137.

Embodiment 155. The use according to any one of Embodiments 152 to 154, wherein the acetonitrile is obtained according to the process according to any one of Embodiments 1 to 126.

Embodiment 156. The use according to any one of Embodiments 152 to 155, wherein the acetonitrile is used as a washing agent, and/or reaction solvent, and/or a diluent in the oligonucleotide synthesis.

Embodiment 157. The use according to any one of Embodiments 152 to 156, wherein the oligonucleotide synthesis is solid phase oligonucleotide synthesis.

Embodiment 158. The use according to any one of Embodiments 152 to 157, wherein the oligonucleotide is RNA.

Embodiment 159. The use according to any one of Embodiments 152 to 157, wherein the oligonucleotide is DNA.

Embodiment 160. The use according to any one of Embodiments 152 to 157, wherein the oligonucleotide comprises both RNA and DNA.

Embodiment 161. The use according to any one of Embodiments 152 to 160, wherein the oligonucleotide is a modified oligonucleotide.

Embodiment 162. The use according to Embodiment 161, wherein the modification is at the 2′ position of the sugar moiety and is selected from the group consisting of 2′-F, 2′-OMe, 2′-MOE, and 2′-amino, or wherein the oligonucleotide comprises a PMO, a LNA, a PNA, a BNA, or a SPIEGELMER.

Embodiment 163. The use according to any one of Embodiments 161 and 162, wherein the modification is in the nucleobase and is selected from the group consisting of a 5-methyl pyrimidine, a 7-deazaguanosine and an abasic nucleotide.

Embodiment 164. The use according to any one of Embodiments 161 to 163, wherein the modification is in the backbone and is selected from the group consisting of phosphorothioate, phosphoramidate and phosphorodiamidate.

Embodiment 165. The use according to any one of Embodiments 161 to 164, wherein the oligonucleotide is selected from an antisense oligonucleotide, an aptamer, siRNA, miRNA, and a gapmer.

Embodiment 166. The use according to any one of Embodiments 161 to 165, wherein the oligonucleotide is an antisense oligonucleotide.

Embodiment 167. The use according to any one of Embodiments 161 to 165, wherein the oligonucleotide is an siRNA.

Embodiment 168. The use according to any one of Embodiments 158 to 167, wherein the oligonucleotide is 10 to 200 nucleotides long.

Embodiment 169. The use according to any one of Embodiments 158 to 168, wherein the oligonucleotide is 18, 19, 20, 21, 22, 23, 24, 25, 26, 27, 28, 29 or 30 nucleotides long.

Embodiment 170. The use according to any one of Embodiments 158 to 169, wherein the oligonucleotide is an 18-mer, 19-mer, 20-mer, 21-mer, 22-mer, 23-mer, 24-mer, 25-mer, 26-mer, 27-mer, 28-mer, 29-mer or a 30-mer.

Embodiment 171. The use according to any one of Embodiments 158 to 170, wherein the oligonucleotide is a therapeutic oligonucleotide.

Embodiment 172. The use according to any one of Embodiments 158 to 171, wherein the oligonucleotide is a single stranded therapeutic oligonucleotide.

Embodiment 173. The use according to any one of Embodiments 158 to 171, wherein the oligonucleotide is a double stranded therapeutic oligonucleotide.

Embodiment 174. The use according to any one of Embodiments 152 to 158, 161 to 166, 168 to 171 and 173, wherein the oligonucleotide is inclisiran.

Embodiment 175. The use according to any one of Embodiments 152 to 158, 161 to 165 and 167 to 172, wherein the oligonucleotide is pelacarsen.

Embodiment 176. The use according to any one of Embodiments 152 to 175, wherein the oligonucleotide of the oligonucleotide synthesis waste is defined according to any one of Embodiments 109 to 126.

Embodiment 177. The use according to any one of Embodiments 152 to 176, wherein the oligonucleotide is produced at gram or kilogram scale, or greater.

### EXAMPLES

The following examples are provided for illustrative purposes.

### Example 1

A number of waste products are produced from the oligonucleotide synthesis process. The waste products are collected into three streams. One of these streams, labelled organic waste contains the majority of the acetonitrile suitable for regeneration. The majority of this stream comprises the numerous acetonitrile washes applied after each reaction step in the process to form an oligonucleotide. The remaining two streams are less suitable for acetonitrile regeneration. One of these streams is labelled chlorinated waste, and contains primarily dichloroacetic acid and toluene, while the other stream is labelled halogenated waste and contains primarily pyridine, iodine and water. Both of these streams contain very little acetonitrile and are less suitable for regenerating acetonitrile than the organic waste stream. Due to some back mixing during the reaction, the segregation of these streams is imperfect and it is not unusual to find for example some pyridine, toluene or water in the organic waste stream.

A number of batch experiments were conducted combining varying amounts of each of the above waste streams to test the principle of regeneration of acetonitrile employing batch distillation.

The batch distillation unit used for the experiments is comprised of a 1 L jacketed glass reactor equipped with an agitator, a head plate containing a number of access points for filling the reactor with waste liquid and fixing measuring probes to the reactor such as temperature probes and pH meters. A 500 mm tall packed-column with an internal diameter of 30 mm is fixed to the head plate. The packing in the column is a structured packing type manufactured by Sulzer Chemtec (DX packing). The packing yields between 16 and 22 theoretical stages depending on the operating conditions employed during these experiments. At the top of the packed column a condenser is located supplied by a 5° C. glycol-water mixture to condense vapours emanating from the top of the packed section. A reflux controller is located at the base of the condenser that controls the fraction of condensate recycled to the packing and the fraction that is removed as product to a distillate collection vessel. A vacuum pump is connected to the unit to control the operating pressure on the condenser zone.

The operating procedure for the batch experiments comprises the following steps.
1. Intertisation of the distillation unit with nitrogen.
2. Turning coolant flow on to the condenser at a temperature between 0° C. and 5° C.
3. Setting the reflux controller to total reflux.
4. Charging of the distillation unit with the solution to be distilled through an access point in the headplate.
5. Switching on the agitator and initial heating of the jacket to 40° C.
6. Turning on the vacuum pump and drawing down the pressure in the column to the desired setpoint.
7. Increasing the jacket temperature until the solution is seen to boil.
8. Waiting until all parts of the column contain distillate and the temperatures in the column have stabilized at constant values.
9. Changing the reflux ratio to the desired value and removing distillate from the system.
10. Increasing the jacket temperature to reach the desired distillate take-off rate.
11. Collecting distillate in fractions.

During a batch distillations, a number of distillate fractions are removed during each experiment and stored in bottles for analysis. The contents of the reactor are allowed to concentrate and only at the end is a sample of the final bottoms residue taken for analysis. The distillation is continued until 80 to 85% of the original charge to the reactor is distilled into fractions.

Table 1.1 shows the fractions of each waste type combined to create the batch charge for each experiment. For example, in Run 1.1, all waste streams are combined to create the feed, while in Run 1.3, all of the organic waste stream and only 20% of the available chlorinated stream are combined to form the batch feed.

Each distillation experiment in table 1.1 is conducted according to the procedure outlined above. Table 1.2 gives some details of the operating conditions of each experiment. Apart from Run 1.1 which was conducted at 400 mbar, all other experiments are conducted at a pressure of 200 mbar. The reflux ratio is at a value of 3 from Run 1.1 through to Run 1.6 and then reduced to measure the effect of lower reflux ratios on the purity of the distilled product. In table 1.2, is also given the number of theoretical separation stages yielded by the hydraulic conditions and the characteristics of the column packing during each experiment.

Table 1.1 shows the composition of each batch charge to the distillation unit as measured by gas chromatography and given in area percent (area %). The concentration of toluene and pyridine are in proportion to the amount of the chlorinated and halogenated waste added to the feed respectively. In Run 1.2, where none of the chlorinated and halogenated waste is added to the feed, there is still residual contamination of both toluene and pyridine in the feed.

In table 1.2, is shown the water content in the feed to each experiment, as well as the water content of the initial and final distillate fractions and the water content of the residue remaining in the batch reaction at the end of the experiment. The experiments which included portions of the halogenated waste stream show higher levels of water in the feed stream and in the distillate and final bottoms product as expected based on the presence of water in the halogenated waste.

Also shown in table 1.2, are the pH values of the same streams as above for the water content. Table 1.2 also shows the experimental time, the calculated number of stages and the operating pressure.

TABLE 1.1
Feed structure and composition of the solution charged to each batch distillation

| | | | | Feed Composition [Area Averaged] | | | | | | |
|---|---|---|---|---|---|---|---|---|---|---|
| | Fractions in Feed | | | | | | | 1- | Acetic | |
| | Organic | Chlorination | Halogenation | Acetonitrile | Toluene | Lutidine | Pyridine | Methylimidazole | anhydride | Other |
| Run | [fr wste] | [fr wste] | [fr wste] | [Area %] | [Area %] | [Area %] | [Area %] | [Area %] | [Area %] | [Area %] |
| 1.1 | 100% | 100% | 100% | 55.21 | 33.24 | 5.28 | 5.12 | — | 0.02 | 1.13 |
| 1.2 | 100% | — | — | 85.81 | 0.02 | 12.93 | 0.03 | — | 0.11 | 1.11 |
| 1.3 | 100% | 20% | — | 75.78 | 12.43 | 10.96 | 0.02 | — | 0.09 | 0.72 |
| 1.4 | 100% | — | 20% | 84.30 | 0.02 | 12.37 | 2.15 | — | 0.10 | 1.06 |
| 1.5 | 100% | — | 100% | 79.62 | 0.03 | 10.46 | 8.85 | — | 0.10 | 0.94 |
| 1.6 | 100% | 100% | | 55.10 | 37.69 | 5.66 | 0.01 | — | 0.02 | 1.52 |
| 1.7 | 100% | 20% | 20% | 72.45 | 14.82 | 9.69 | 2.29 | 0.25 | — | 0.50 |
| 1.8 | 100% | 20% | 20% | 70.69 | 14.53 | 9.42 | 2.18 | 0.27 | — | 2.91 |
| 1.9 | 100% | 10% | 100% | 73.51 | 6.88 | 8.76 | 10.06 | 0.38 | — | 0.41 |

TABLE 1.2
Water content and pH of feed, bottoms and distillate streams, and operating conditions for each experiment

| | Water Composition | | | | | | | | Operational | | | |
|---|---|---|---|---|---|---|---|---|---|---|---|---|
| | | | Initial | Final | pH of Streams | | | | | Theoretical | | |
| | | | distillate | distillate | | | Initial | Final | | Stages of | | RR |
| | Feed | Bottoms | fraction | fraction | | | distillate | distillate | Time | separation | Pressure | [sec > |
| Run | [ppm] | [ppm] | [ppm] | [ppm] | Feed | Bottoms | fraction | fraction | [hrs] | [theor] | [mbar] | sec] |
| 1.1 | 4700 | 320 | — | — | — | 6.04 | 7.25 | 6.8 | 11.8 | 21.6 | 400 | 3 |
| 1.2 | 330 | 110 | 2709 | 729 | 7.2 | 7.3 | 6.6 | 6.8 | 5.2 | 19.9 | 200 | 3 |
| 1.3 | 239 | 215 | 3235 | 1193 | 6.8 | 6.3 | 6.8 | 6.2 | 5.0 | 19.8 | 200 | 3 |
| 1.4 | 1907 | 90.1 | 8643 | 1243 | 7.2 | 7.2 | 8.4 | 8.2 | 5.5 | 20.0 | 200 | 3 |
| 1.5 | 6845 | 305 | 33762 | 1042 | 7.2 | 7.2 | 6.9 | 7 | 5 | 19.9 | 200 | 3 |
| 1.6 | 288.6 | 326.7 | 903.1 | 7680 | 5.7 | 5.8 | 7 | 8 | 4.8 | 20.26 | 200 | 3 |
| 1.7 | 1622 | 124.1 | 6442 | | 6.7 | 6.7 | 7.3 | 8.7 | 1.6 | 16.139 | 200 | 0.5 |
| 1.8 | 1813 | 582 | 4198 | 673 | 6.7 | 7 | 7.6 | 6.4 | 4.8 | 19.867 | 200 | 0.33 |
| 1.9 | 7300 | 2700 | 48900 | 3200 | — | — | — | — | 4.3 | 19.614 | 200 | 3 |

TABLE 1.3
Averaged distillate composition, and yield and
overall mass balance for each experiment

| | Distillate Composition [Area Averaged] | | | | | Yield/MB | |
|---|---|---|---|---|---|---|---|
| | Acetonitrile | Toluene | Lutidine | Pyridine | Other | MB | Yield |
| Run | [Area %] | [Area %] | [Area %] | [Area %] | [Area %] | [wt %] | [Area %] |
| 1.1 | 67.16 | 32.71 | — | <10 ppm | 0.13 | 90.0% | 97.1% |
| 1.2 | 99.81 | 0.18 | — | — | 0.01 | 93.4% | 94.8% |
| 1.3 | 84.95 | 15.05 | — | — | 0.00 | 92.8% | 96.5% |
| 1.4 | 99.97 | 0.03 | — | <10 ppm | 0.00 | 90.1% | 94.5% |
| 1.5 | 99.96 | 0.02 | — | <50 ppm | 0.02 | 94.1% | 97.7% |
| 1.6 | 69.37 | 30.63 | — | | 0.00 | 91.4% | 96.9% |
| 1.7 | 81.98 | 17.72 | 0.00 | 0.19 | 0.11 | 91.9% | 95.9% |
| 1.8 | 79.32 | 17.30 | 0.18 | 0.01 | 3.19 | 96.1% | 92.8% |
| 1.9 | 91.26 | 8.71 | — | — | 0.02 | 94.6% | 96.1% |

In table 1.3, the average composition of the distillates across all fractions from each experiment is given. The mass balance from each step as well as the overall yield of acetonitrile from each experiment is calculated.

The results above demonstrate the following aspects of batch distillation of wastes emanating from the synthesis of oligonucleotides.
1. In the absence of chlorinated waste stream in the starting solution an acetonitrile purity in excess of 99% is achieved. This is due to the presence of a binary toluene/acetonitrile azeotrope resulting in the proportion of toluene present in the distillate being proportional to the amount present in the feed.
2. At a reflux ratio of 3, even when all of the halogenated waste stream is included in the feed, only trace quantities of pyridine are measured in the distillate and a purity in excess of 99% can be achieved in the distillate. If the reflux ratio is reduced to below unity, the concentration of pyridine in the distillate increases.
3. As expected, water is at its highest concentration in feed, distillate and bottoms when the halogenated waste is included in the feed mixture during the batch charge. The water present in the feed will concentrate in the distillate due to the presence of a number of low boiling binary azeotropes between acetonitrile and water and toluene and water.

The above examples demonstrate the importance of segregating waste streams to present an initial feed material with a high concentration of acetonitrile to a process where acetonitrile is regenerated.

The above example also demonstrates that when the feed is contaminated with toluene and water, supplementary separation steps are necessary to lower the concentration of both to be able to meet a purity specification in excess of 99.9% acetonitrile and a water specification below 30 parts per million.

In addition, the above examples demonstrate the following.
1. The utility of using a packed column to improve separation of compounds that are difficult to separate due to low relative volatility between these compounds and acetonitrile.
2. The utility of reflux ratio as an operating parameter to improve the purity of distilled acetonitrile.

Having identified the need to employ further processing stages to separate acetonitrile from azeotrope forming impurities and to de-water the final product, the following examples employ continuous distillation as opposed to batch distillation where specific separation strategies can be used and run in steady-state to maximize yield and purity of the regenerated product.

### Example 2

Table 2.1 below shows the results of a number of continuous experiments using different combinations of waste streams resulting from the oligonucleotide production process. The same equipment as described in Example 1 was used for these experiments, with the exception that the batch reactor was replaced with a jacketed wiped-film evaporator. The wiped-film evaporator is a vertical device with an aspect ratio of approximately eight-to-one and an internal diameter of 50 mm. A central rotating shaft has plastic wipers attached to it that functions to distribute the incoming fluid as a thin layer on the internal surface of the device. The central shaft has a variable speed motor but is typically operated between 120 and 160 revolutions per minute. The fraction of the feed evaporated in the wiped-film evaporator is a function of the temperature of the fluid circulating through the jacket, the rotational speed of the shaft, the feed rate and the operating pressure in the column. The residence time in the wiped-film evaporator is in the order of several seconds, and the hold-up is in the order of several grams.

The vapour generated in the evaporator flows upwards and into the packed column situated on top of the evaporator. A condenser at the top of the packed column is equipped with a condenser and a reflux splitter. A fraction of the condensate is returned to the top of the packing and the remainder is returned as product to an external collection vessel.

The feed compositions for each experiment are shown in Table 2.1. In experiments where the chlorinated waste is added to the organic feed, the concentration of toluene is elevated in the total feed compared to the experiments where it is not added. In experiments where the halogenated waste is added to the organic feed, the concentration of pyridine is elevated. In feeds where both chlorinated and halogenated wastes are added to the organic feed, both toluene and pyridine have a higher overall concentration in the feed. Only 10% of the chlorinated and halogenated waste streams were added to the organic waste stream, as the previous experiments had demonstrated the issues associated with toluene and pyridine in the feed stream. For this reason, only 10% was added to measure the effect of contamination.

The operating conditions for the experiments are shown in Table 2.2. All experiments were conducted at a constant pressure of 200 mbar. Experiments 2.1 to 2.4 are conducted at a reflux ratio of 3, while experiments 2.5 and 2.6 are run at a reflux ratio of 4. Runs 2.5 and 2.6 are repeats of experiments 2.3 and 2.4 to demonstrate the benefit of increasing the reflux ratio in order to reduce the levels of pyridine contamination in the distillate.

TABLE 2.1
Fraction of waste streams used in the feed to each experiment and component concentration

| | | | | Feed Composition | | | | | | | | |
|---|---|---|---|---|---|---|---|---|---|---|---|---|
| | | | | | | | | | | | 2,6- | |
| | Fractions in Feed | | | MeCN | | | | Ac₂O | Pyridine | Toluene | Lutidine | Other |
| | Organic | Chlorinated | Halogenated | [Area | ACN | EtOAc | DIA | [Area | [Area | [Area | [Area | [Area |
| Run | [fr wste] | [fr wste] | [fr wste] | %] | [ppmA] | [ppmA] | [ppmA] | %] | %] | %] | %] | %] |
| 2.1 | 100% | | | 87.12 | | — | 302 | 0.09 | 0.03 | 0.02 | 11.52 | 1.19 |
| 2.2 | 100% | 10% | | 81.03 | 30 | 45 | — | 0.12 | 0.03 | 7.85 | 10.18 | 0.79 |
| 2.3 | 100% | | 10% | 86.56 | 49 | — | — | 0.1 | 1.76 | 0.1 | 10.84 | 0.63 |
| 2.4 | 100% | 10% | 10% | 80.07 | 25 | — | — | 0.12 | 1.25 | 7.22 | 10.58 | 0.75 |
| 2.5 | 100% | | 10% | 85.75 | 11 | — | — | 0.13 | 1.27 | 0.02 | 11.81 | 1.02 |
| 2.6 | 100% | 10% | | 79.74 | 17 | — | — | 0.13 | 1.23 | 6.87 | 10.91 | 1.13 |

MeCN = Acetonitrile;
EtOAc = Ethyl acetate;
DIA = Diisopropylamine;
ACN = acrylonitrile;
Ac₂O = acetic anhydride

TABLE 2.2
Operating conditions, fractionation and
yield of acetonitrile in the distillate

| Operating Conditions | | | | | | | | |
|---|---|---|---|---|---|---|---|---|
| | | | Feed | Fractionation & Staging | | | | |
| | Druck | | [gr/ | Dist | Sumpf | Dist | | |
| Run | [mbar] | RR | hr] | [gr] | [gr] | [Frac] | Stages | Yield |
| 2.1 | 200 | 3 | 200 | 83 | 17 | 79.50% | 12.7 | 92.20% |
| 2.2 | 200 | 3 | 213 | 139.1 | 49.1 | 64.70% | 12.4 | 88.70% |
| 2.3 | 200 | 3 | 140 | 102.9 | 37 | 64.00% | 14.4 | 88.90% |
| 2.4 | 200 | 3 | 202 | 131.9 | 36.6 | 72.30% | 12.7 | 91.40% |
| 2.5 | 200 | 4 | 107 | 137.6 | 28.1 | 79.60% | 15.6 | 95.10% |
| 2.6 | 200 | 4 | 103 | 130.1 | 33.5 | 74.30% | 13.4 | 92.30% |

In experiments where the chlorinated waste is included in the feed, the concentration of toluene in the distillate is largely unchanged even when the reflux ratio is increased from 3 to 4, while the concentration of pyridine is reduced when the reflux ratio is increased. The concentration of 2,6-Lutidine is also reduced when the reflux ratio is increased. The concentration of low boilers such as diisopropylamine and acrylonitrile partition between distillate and bottoms. The overall low concentration of both makes an exact quantification of partitioning difficult, but even at a reflux ratio of 4, both are still present in the distillate indicating the need for further stages of purification to arrive at a acetonitrile purity in excess of 99.90 area %.

TABLE 2.3
Composition of distillate from each experiment
Distillate Composition

| | | | Ethyl | | Acetic | | | 2,6- | |
|---|---|---|---|---|---|---|---|---|---|
| | Acetonitrile | Acrylonitrile | acetate | Diisopropylamine | anhydride | Pyridine | Toluene | Lutidine | Other |
| Run | [Area %] | [ppmA] | [ppmA] | [ppmA] | [Area %] | [ppmA] | [Area %] | [Area %] | [Area %] |
| 2.1 | 99.91 | — | — | 419 | — | — | 0.02 | — | 0.02 |
| 2.2 | 89.91 | 6 | 26 | 23 | — | — | 10.09 | — | — |
| 2.3 | 99.80 | 44 | 98 | 112 | — | 415 | 0.13 | — | — |
| 2.4 | 90.49 | 6 | 25 | 21 | — | 110 | 9.49 | — | — |
| 2.5 | 99.94 | 9 | 24 | 261 | — | 4 | 0.03 | — | — |
| 2.6 | 91.08 | 6 | 24 | 22 | — | — | 8.92 | — | — |

TABLE 2.4
Composition of bottoms from each experiment
Bottoms Composition

| | | | Ethyl | | Acetic | | | 2,6- | |
|---|---|---|---|---|---|---|---|---|---|
| | Acetonitrile | Acrylonitrile | acetate | Diisopropylamine | anhydride | Pyridine | Toluene | Lutidine | Other |
| Run | [Area %] | [ppmA] | [ppmA] | [ppmA] | [Area %] | [Area %] | [Area %] | [Area %] | [Area %] |
| 2.1 | 41.36 | 132 | — | 419 | 0.73 | 0.21 | 0.01 | 52.68 | 4.96 |
| 2.2 | 39.33 | 102 | 68 | — | 0.85 | 0.21 | 1.71 | 54.29 | — |
| 2.3 | 34.80 | 174 | — | 837 | 0.48 | 9.03 | 0.03 | 52.94 | — |
| 2.4 | 30.81 | 109 | — | — | 0.77 | 7.05 | 2.13 | 55.76 | — |
| 2.5 | 25.01 | 151 | — | 244 | 0.85 | 6.96 | 0.01 | 61.88 | — |
| 2.6 | 29.64 | 116 | — | — | 0.81 | 6.98 | 1.73 | 57.17 | — |

Similar to the batch experiments, the distillate produced from continuous distillation has a proportional concentration of toluene as measured in the feed. Even in experiments where the chlorinated waste is not combined into the feed, toluene is still present in the distillate at low levels also proportional to the feed. The distillate is also contaminated with low level of light boilers such as diisopropylamine and acrylonitrile. Two of the experiments exhibit a purity in excess of 99.90 area % on an water-free basis only, with yields in excess of 90 wt %, however the levels of impurities are not acceptable, and there is also a quantity of water in the distillate far in excess of 30 ppm by weight.

Similar conclusions as drawn for the batch distillations in Example can be drawn for this example. In addition, the benefit of using continuous distillation to maintain a product stream with a fixed purity can be appreciated. The inherent advantages of continuous processing such as low capital investment, less down time and higher yield can be appreciated.

### Example 3

In this example, a feed is processed through all stages of the organic side of the process as depicted in claim 1, and subsequently dewatered to produce a product suitable for recycling to the oligonucleotide synthesis reaction. The organic waste streams from a reaction sequence are processed through four processing steps to purify acetonitrile. A description of the steps is given in table 1.

TABLE 3.1
Composition of bottoms from

| Step | Description |
|---|---|
| Distillation 3.1 | The initial feed is distilled to separate heavy boilers such as 2,6-Lutidine, |
| (Heavy | DMF, acetic acid, pyridine, dichloroacetic acid from light boilers such as |
| distillation) | acetonitrile, toluene, diisopropylamine, acrylonitrile in high yield |
| Distillation 3.2 | The distillate from the previous step is processed to separate light boilers or |
| (Light | compounds forming low boiling azeotropic mixtures with water and/or |
| distillation) | acetonitrile from acetonitrile/water. For this purpose, water is added to the |
| | feed as an entrainer to form the azeotropes and ensure the removal of all of |
| | the toluene and diisopropylamine and the majority of the acrylonitrile. The |
| | amount of water is limited to twenty times the concentration of toluene in the |
| | feed to yield low water content in the acetonitrile product. |
| Distillation 3.3 | The bottoms product from the previous step is processed to remove as much |
| (Azeotropic | water as possible in the form of an acetonitrile/water azeotrope. |
| distillation) | |
| Drying 3.4 | The bottoms product from the previous step is pumped through a column |
| | filled with a molecular sieve to remove residual water from acetonitrile to |
| | below 30 ppm. |

The equipment used for the regeneration is the same as described in examples 1 and 2 above.

The general operating conditions and parameters of operation for each stage is listed in Table 3.2 (Distillation 3.1 to 3.3) and Table 2 (Drying step 3.4).

TABLE 3.2
Operating conditions and parameters for Distillations 3.1 to 3.3

| Distillation column | | | | |
|---|---|---|---|---|
| parameters | Units | Heavy | Light | Azeotropic |
| Jacket temperature WFE | [° C.] | 78-80 | 96.5-98 | 96.5-98 |
| Condenser temperature | [° C.] | 4 | 4 | 4 |
| Condenser pressure | [mbar] | 200 | 980 | 980 |
| Wiped film cage speed | [rpm] | 150 | 165 | 165 |
| Feed stage | | 13 | 2 | 2 |
| Product stage | | 1 | 14 | 14 |
| Feed temperature | [° C.] | 25 | 80 | 25 |
| Distillate to product | [secs] | 5 | 5 | 5 |
| Distillate to reflux | [secs] | 25 | 60 | 60 |

TABLE 3.3
Operating conditions and parameters for step 3.4

| Drying column parameters | Units | Drying |
|---|---|---|
| Reactor length | [cm] | 8 |
| Reactor internal diameter | [mm] | 35 |
| Mass molecular sieve | [gr] | 50 |
| Reactor voidage | [m3/m3] | 0.38 |
| Feed rate | [mL/min] | 15.6 |

The feed is available in two portions, and the composition of each portion is shown in table 3.4.

TABLE 3.4
Feed composition in area % or ppm (area)

| | | | | | Acetic | | | | 2,6- | |
|---|---|---|---|---|---|---|---|---|---|---|
| | MeCN | ACN | DMF | DIA | acid | DCA | Pyridine | Toluene | Lutidine | Water |
| | [Area %] | [ppmA] | [ppmA] | [ppmA] | [Area %] | [ppmA] | [A %] | [ppmA] | [wt %] | [wt %] |
| Feed 1 | 90.17 | 28 | 16 | | 0.3072 | | 4.41 | 1187 | 4.30 | <1 |
| Feed 2 | 89.83 | 392 | 12 | | 0.1291 | | 4.46 | 1248 | 3.89 | <1 |

MeCN = Acetonitrile;
DMF = Dimethylformamide;
DIA = Diisopropylamine;
ACN = acrylonitrile;
DCA = Dichloro acetic acid

Table 3.5 shows the composition of distillate and bottoms fractions from distillation 3.1. Feed 3 corresponds to Dist 3.1 to 3.3 and Btms 3.1, and Feed 2 corresponds to Dist 3.4 to 3.6 and Btms 3.2. The reflux ratio of the step is 5 and the number of theoretical separation stages is approximately 14. Under these conditions, the purity of acetonitrile increases from approximately 90.0 Area % (Feed) to 99.80 Area % (Distillate). Major impurities in the distillate are acrylonitrile, toluene, diisopropylamine as well as small quantities of pyridine. Practically all of the heavy boilers (acetic acid, dichloroacetic acid, pyridine, 2,6-Lutidine) are segregated to the bottom product. 22% of the feed is separated to the bottoms and 78% to the distillate. The overall mass balance for this step is 99.2 wt % and the yield of acetonitrile was 85.6 wt % (also summarized in Table 3.6).

TABLE 3.5
Distillate and bottoms composition from step 1

| | Dist | Dist | Dist | Dist | Dist | Dist | Btms | Btms | |
|---|---|---|---|---|---|---|---|---|---|
| | 3.1 | 3.2 | 3.3 | 3.4 | 3.5 | 3.6 | 3.1 | 3.2 | Units |
| Acetonitrile | 99.83 | 99.83 | 99.78 | 99.78 | 99.77 | 99.78 | 61.5 | 54.84 | [wt %] |
| Acrylonitrile | 20 | 28 | 439 | 457 | 482 | 453 | 91 | 128 | [ppm] |
| Dimethylformamide | | | | | | | | | [ppm] |
| Diisopropylamine | 0 | 0 | 35 | 37 | 37 | 37 | | | [ppm] |
| Acetic acid | | | | | | | 0.9925 | 0.6785 | [wt %] |
| Dichloro acetic acid | | | | | | | 7 | 0 | [ppm] |
| Pyridine | 18 | 15 | 26 | 44 | 25 | 51 | 17.82 w | 20.91 w | [ppm] |
| Toluene | 1591 | 1593 | 1637 | 1589 | 1687 | 1575 | 291 | 325 | [ppm] |
| 2,6-Lutidine | | | | | | | 16.88 | 18.13 | [wt %] |
| Water | 2.04% | 2.04% | 2.07% | 2.07% | 2.24% | 2.12% | | | [wt %] |

TABLE 3.6
Summary of fractions to distillate and bottoms,
mass balance and yield of each step

| Phase | Heavy | Light | Azeotropic | Drying |
|---|---|---|---|---|
| Distillate fraction | 78% | 15.82% | 11.49% | — |
| Bottoms fraction | 22% | 81.09% | 86.85% | — |
| Mass balance | 99.2% | 96.9% | 98.3% | 96.1% |
| Yield | 85.6% | 83.8% | 89.0% | 96.1% |

The water content in the distillate stream from Dist 3.1 was adjusted by adding water to increase the concentration to approximately 2 wt %. The water was added to act as entrainer to remove toluene in the next step.

The distillate from step 3.1 is processed according to the operating conditions and parameters listed in Table 3.2. Four bottoms fractions and a single distillate fraction are collected from this step and the compositions are shown in table 3.7.

Approximately 98% of the toluene, 50% of the acrylonitrile and 100% of the diisopropylamine are separated to the distillate along with some acetonitrile and the vast majority of the water. All of the pyridine in the feed is found in the bottoms product from the column. Overall, 15.82% of the feed was separated to the distillate and 81.09% to the bottoms. The overall mass balance for this step was 96.9 wt % and the yield of acetonitrile was 83.8 wt % (also summarized in Table 3.7).

TABLE 3.7
Distillate and bottoms composition from step 2

| | Btms 1 | Btms 2 | Btms 3 | Btms 4 | Dist 1 | |
|---|---|---|---|---|---|---|
| Acetonitrile | 99.92 | 99.93 | 99.94 | 99.92 | 94.83 | [Area %] |
| Acrylonitrile | 40 | 317 | 293 | 112 | 833 | [ppm] |
| Diisopropylamine | | | | | 103 | |
| Pyridine | 23 | 48 | 42 | 33 | | [ppm] |
| Toluene | 240 | 307 | 241 | 696 | 0.974 w % | [ppm] |
| Water | 1368.6 | 1702.25 | 1058.5 | 11031 | 11.53 w % | [ppm] |

The distillate from Distillation 3.2 is processed according to the operating conditions and parameters listed in Table 3.2. Two bottoms fractions and two distillate fraction are collected from this step and the compositions are shown in table 3.8.

The water content in the product from this step is reduced from an average of 3786 ppmW to 378 ppmW in the bottoms product. Along with the water, toluene and acrylonitrile are further reduced while traces of diisopropylamine were also separated. All of the pyridine in the feed is found in the bottoms product from the column. Overall, 11.49% of the feed is separated to the distillate and 86.85% to the bottoms. The overall mass balance for this step is 98.3 wt % and the yield of acetonitrle was 89 wt % (also summarized in Table 3.6).

TABLE 3.8
Distillate and bottoms composition from step 3

| | Btms 1 | Btms 2 | Dist 1 | Dist 2 | |
|---|---|---|---|---|---|
| Acetonitrile | 99.96 | 99.94 | 99.79 | 99.83 | [Area %] |
| Acrylonitrile | 79 | 304 | 212 | 585 | [ppmA] |
| Pyridine | 59 | 15 | | | [ppmA] |
| Diisopropylamine | | | 33 | | [ppmA] |
| Toluene | 204 | 260 | 1803 | 1066 | [ppmA] |
| Water | 576 | 199 | 8782 | 13130 | [ppmW] |

The bottom product from distillation 3 is pumped through a molecular sieve column according to the operating conditions and parameters in Table 3.3. The product is collected in four fractions, the compositions of which are shown in table 3.9.

TABLE 3.9
Composition and purity of product from drying column

| Fraction | Product 1 | Product 2 | Product 3 | Product 4 | |
|---|---|---|---|---|---|
| Acetonitrile | 99.97 | 99.96 | 99.96 | 99.97 | [Area %] |
| Acrylonitrile | 93 | 226 | 224 | 125 | [ppmA] |
| Pyridine | 38 | 46 | 38 | 20 | [ppmA] |
| Toluene | 193 | 149 | 147 | 181 | [ppmA] |
| Water | 29.2 | 24.8 | 26.5 | 26.4 | [ppmW]* |

*measured by Karl-Fisher water analysis

This example shows that it is possible to consistently regenerate acetonitrile from an initial purity of 90 area % to a purity in excess of 99.90 area % and also to reduce the water content to below 30 ppmW.

The quantity of water added to the feed to distillation 2 is well below the optimal amount to remove low boiling azeotropic mixtures from the feed. In general, more water in the feed will increase the removal efficiency of toluene, acrylonitrile and diisopropylamine. A quantity of water in excess of the concentration that yields a water concentration from the bottom of the column in excess of the azeotropic composition at the column operating conditions is preferred. However, a water concentration below this is also acceptable but less robust.

It will be understood that the limited number of stages available in a laboratory column are not sufficient to completely remove the residual impurities shown in Table 3.9. More stages of separation combined with more water will allow the concentrations of light boiling azeotrope former shown in table 3.9 to be reduced well below the values shown in table 3.9.

### Example 4

In this example, acetonitrile is processed through all stages of the organic side of the process, as well as the aqueous side of the process, the products of which are combined and subsequently dewatered to produce a product suitable for recycling to the oligonucleotide synthesis reaction.

The aqueous stream is processed through two stages of distillation (distillation 4.1) and then combined with the product from distillation 4.2 as described in table 4.1. From here the product is processed through two stages of dewatering (distillation 4.3 and 4.4) to arrive at an product that is in specification.

TABLE 4.1
Overview of processing steps

| Step | Description |
|---|---|
| Distillation 4.1 | The aqueous waste stream containing 8.34 wt % acetonitrile and 0.51 wt % |
| (Aqueous | ethanol is distilled to separate acetonitrile from water. This step is described |
| Heavy | in table 4.2. |
| distillation) | |
| Distillation 4.2 | The organic feed is distilled to separate heavy boilers such as 2,6-Lutidine, |
| (Organic | DMF, acetic acid, pyridine, dichloroacetic acid from light boilers such as |
| Heavy | acetonitrile, toluene, diisopropylamine, acrylonitrile in high yield. Results are |
| distillation) | shown in tables 4.3. |
| Distillation 4.3 | The distillate from the organic distillation heavy is processed to separate |
| (Organic light | light boilers or compounds forming low boiling azeotropic mixtures with |
| distillation) | water and/or acetonitrile from acetonitrile. For this purpose, water is added |
| | to the feed as an entrainer to form azeotropes and ensure the removal of all |
| | of the toluene and diisopropylamine and the majority of the acrylonitrile. The |
| | amount of water is limited to twenty times the concentration of toluene in the |
| | feed to yield low water content in the acetonitrile product. |
| Distillation 4.4 | The product from the aqueous distillation heavy and the organic light |
| (Combined | distillation are combined and distilled to reduce the concentration of water |
| azeotropic | in the product by evaporating a water rich fraction from the feed. These |
| distillation) | results are shown in table L and f. |
| Distillation 4.5 | The bottoms product from distillation 4.4 is distilled in its entirety in this step. |
| | As the actual process comprises a distillation step from which the distillate |
| | is the product, this distillation is done to simulate that design. |
| Drying 4.6 | The bottoms product from the combined azeotropic distillation is treated with |
| | molecular sieves to reduce the water content to a value close to 30 ppm. |

TABLE 4.1
Operating conditions, overall stage mass balance and yield per stage

| | Aqueous | Organic | Organic | Combined | Combined | |
|---|---|---|---|---|---|---|
| | Heavy | Heavy | Light | Azeotropic | Distillation | Units |
| Operating Pressure | 450 | 200 | 980 | 980 | 250 | [mbar] |
| Reflux Ratio | 1 | 7 | 12 | 0.33 | 0 | [sec/sec] |
| Total Feed | 3038 | 3079 | 2181 | 2198 | 1220 | [gr] |
| Acetonitrile in Feed | 334 | 2707 | 2174 | 2212 | 1208 | [gr] |
| Distillate | 303 | 2373 | 177 | 746 | 1200 | [gr] |
| Acetonitrile in Distillate | 288 | 2365 | 57 | 673 | 1186 | [gr] |
| Bottoms | 2704 | 559 | 1979 | 1320 | 20 | [gr] |
| Acetonitrile in Bottoms | 2 | 265 | 1978 | 1306 | 20 | [gr] |
| Overall mass balance | 98.98% | 95.23% | 98.89% | 93.99% | 100.0% | [wt %] |
| Acetonitrile yield | 91.08% | 87.40% | 90.99% | 62.49% | 98.23% | [wt %] |

The aqueous acetonitrile containing waste from the process containing 8.34 wt % acetonitrile is processed through the same distillation column as described in example 1, to concentrate the acetonitrile in the feed to a concentration of 91.43 wt % in the distillate, along with 1.95 wt % of ethanol. The product is retained for further processing through azeotropic distillation along with the product from organic light distillation step. The compositions are of feed, distillate and bottoms are shown in table 4.2.

TABLE 4.2
Aqueous distillation stream composition

| | Acetonitrile | Ethanol | Water |
|---|---|---|---|
| | [wt %] | [wt %] | [wt %] |
| Feed | 8.34 | 0.51 | 91.15 |
| Distillate | 91.43 | 1.95 | 6.62 |
| Bottoms | 0.07 | 0.21 | 99.72 |

The organic waste stream is distilled in the continuous distillation column similar to the method described in example 2. The original feed containing 87.19 area % is concentrated to 99.70 area % in the distillate stream. The major impurity in the distillate is toluene with minor quantities of pyridine, acrylonitrile and ethyl acetate. The majority of the heavy boilers are retained in the bottoms product. It is possible to reduce the pyridine concentration in the distillate by either reducing the distillate fraction, increasing the reflux ratio or increasing the number of separation stages in the columns. The compositions are of feed, distillate and bottoms are shown in table 4.3.

TABLE 4.3
Stream concentration to and from the heavy organic distillation step

| | | | | | | | | 2,6- | 1-Methyl |
|---|---|---|---|---|---|---|---|---|---|
| | Acetonitrile | Acrylonitrile | EtOAc | DIA | Pyridine | Toluene | DMF | Lutidine | Imidazole |
| | [wt %] | [ppmW] | [ppmW] | [ppmW] | [wt %] | [wt %] | [ppmW] | [wt %] | [wt %] |
| Feed | 87.19 | 32 | | | 6.46 | 0.23 | 20.82 | 4.40 | |
| Distillate | 99.70 | 38 | 18 | 13 | 80 ppm | 2420 ppm | | | |
| Bottoms | 47.41 | 298 | 22 | | 25.06 | 384.15 | 2076 | 21.95 | 2.95 |

EtOAc = Ethyl acetate;
DMF = Dimethylformamide;
DIA = Diisopropylamine

The concentration of water in the distillate from the heavy distillation stage is 0.28 weight %. In order to remove the toluene present in the distillate during the light distillation phase, water is added to the distillate to reach a ratio of 30 times the concentration of toluene present. This ratio ensures that most of the toluene is purged from the process in the form of a ternary azeotrope with water and acetonitrile. While this ratio is far below the expected ratio that will be used in the large scale process, the absence of a small scale pressure swing distillation plant meant that water in the process had to be minimized to facilitate dewatering using a single azeotropic distillation step coupled with a dewatering step using molecular sieves.

The light distillation step feed and product stream concentrations are summarized in table 4.4. The organic concentration values are stated on a water free basis in the table. All impurities in the feed stream with the exception of pyridine are enriched in the distillate phase and depleted in the bottoms product. All of the diisopropylamine is found in the distillate.

TABLE 4.4
Stream concentration to and from the light organic distillation step

| | Acetonitrile | Acrylonitrile | EtOAc | Diisopropylamine | Pyridine | Toluene | Water |
|---|---|---|---|---|---|---|---|
| | [wt %] | [ppmW] | [ppmW] | [ppmA] | [ppmA] | [ppmW] | [wt %] |
| Feed | 99.70 | 41 | 22 | 13 | 112 | 2730 | 6.07% |
| Distillate | 90.13 | 159 | 193 | 35 | 21 | 92754 | 41.15% |
| Bottoms | 99.95 | 20 | 2 | | 102 | 360 | 3.00% |

The bottom product from the light distillation step is combined with the distillate product from the aqueous heavy distillation step to form the feed to the azeotropic distillation step. The goal of this step is to reduce the water concentration from an initial value of 3.49 wt % to a value amenable to drying in the next step using molecular sieves. In the closed loop recycle process envisaged for large scale regeneration of acetonitrile, the product from this step will be the distillate and not the bottom product. The distillate will be processed in a second distillation stage to produce a pure acetonitrile bottom product and an azeotropic composition for recycle. Due to the unavailability of a pressure swing distillation on a small scale, the azeotropic distillation will instead produce the product as the bottoms product and the distillate which would normally be recycled, will instead leave the mass balance. In order to still achieve the number of purification steps, the bottoms product will be evaporated in batch distillation mode and without a packing column to effect fractionation of the distillate. This ensures that the product distillate has undergone the same number and type of purification stages as in the intended large scale process.

The results of the azeotropic distillation are shown in table 4.5. The water concentration between feed and bottom product has reduced from 3.49 wt % to 0.98 wt %. The concentration of ethanol has reduced from 3393 ppmA to 175 ppm A in the product. The concentration of all other components remains relatively constant. The reduction in ethanol concentration is an important feature of this step in the process as it is detrimental even at very low concentrations to the synthesis reaction. While the concentration in the product distillate reduces to only 175 ppmA, it will be understood that with the limited number of stages available for fractionation in the packed column (approximately 15 stages), further purification is not possible.

TABLE 4.5
Stream concentration to and from the combined azeotropic distillation

| | | | | | | Ethyl | | | Dimethyl |
|---|---|---|---|---|---|---|---|---|---|
| | | Water | Acetonitrile | Ethanol | Acrylonitrile | Acetate | Pyridine | Toluene | Formamide |
| | Quantity | [wt %] | [wt %] | [ppmA] | [ppmA] | [ppmA] | [ppmA] | [ppmA] | [ppmA] |
| Feed | 2197.9 | 3.49 | 99.58 | 3393 | 22 | 17 | 113 | 269 | 182 |
| Distillate | 745.9 | 8.56 | 98.7144995 | 11399 | 46 | 11 | 5 | 1369 | |
| Bottoms | 1319.9 | 0.98 | 99.96 | 175 | 13.3 | 19 | 120.3 | 23 | |

The bottoms from the previous stage is distilled in batch mode to effect the required number of purification stages as described above. The concentration in the feed and product streams are shown in table 4.6. The quality of the feed and distillate are comparable, containing 9800 ppmW of water and the purity of the product on an organic basis is 99.96 area %.

TABLE 4.6
Stream concentration to and from the single stage batch distillation

| | Acetonitrile | Water | Ethanol | Acrylonitrile | Ethylacetate | Pyridine | Toluene |
|---|---|---|---|---|---|---|---|
| | [wt %] | [ppmW]** | [ppmA*] | [ppmA] | [ppmA] | [ppmA] | [ppmA] |
| Feed | 99.96 | 9800 | 175 | 13.3 | 19 | 120.3 | 23 |
| Distillate | 99.83 | 9870 | 93 | 25 | 42 | 128 | 24 |
| Bottoms | 99.95 | 5600 | 83 | 12 | 42 | 206 | 15 |

*ppmA = parts per million on an area basis as measured by GC.
**ppmW = parts per million on a weight basis

As a final step, the product is dried by adding 300 grams of molecular sieve to 1200 grams of distillate product and allowed to settle for 16 hours after which the water concentration had reduced to 25 ppmW.

The above example demonstrates the feasibility of all steps in the process on both the aqueous and organic sides of the process to produce a product with a purity in excess of 99.90 area % and a water content below 30 ppmW.

### Example 5

To demonstrate that the process could be implemented on a larger scale, a piloting exercise was conducted. A distillation column with the properties listed in table 5.1 was used to demonstrate all the steps in the process.

TABLE 5.1
Properties of the distillation column

| Parameter | Value |
|---|---|
| Column internal diameter (mm) | 50 |
| Column height (m) | 7.5 |
| Column packed height (am) | 6.5 |
| Packing type | Sulzer |
| Theoretical stages | <35 |
| Nominal feed rate range (L/hour) | 3-10 |
| Temperature range (° C.) | 20-200 |
| Pressure range (mbar) | 50-980 |
| Kettle type | Reboiler |
| Heating medium | Silicon oil |
| Condenser type | Total |
| Condenser fluid | Cooled water |
| Feed position | Variable |
| Feed preheating medium | Hot water |

A single column was used to pilot all the distillation columns in the process. To change the number of stages in the column to correspond to the expected design configuration, the amount of packing in the column was physically changed by either removing or adding sections of packing. The feed position was changed depending on which column was being piloted, to a location at the base, the midpoint of the column or higher up on the column. The reflux ratio was controlled using a timed magnetic valve that alternatively returned condensate to the column or diverted it to a distillate receiver.

The column was balanced using weighting scales that were placed under each mass flow stream (feed, distillate, underflow) to and from the process. The time-based weight collected at each location gave the operator sufficient information to calculate the split of the feed between distillate and underflow, and an action could be taken to adjust this split such as increasing the reboiler heat input of changing the reflux ratio. For example, the heat input to the column reboiler could be manipulated at constant reflux ratio to achieve a greater or lesser split between distillate and underflow streams.

The start-up procedure was identical for each column. The feed mixture was charged to the column base at atmospheric conditions. The charged material was then heated and refluxed internally at the desired operating pressure, until the temperature profile in the column converged to steady-state values. Once this was achieved, the distillate withdrawal system was activated to remove top product continuously, while the underflow pump was also started to remove bottom product continuously. The feed pump was also started to introduce fresh feed to the column. The underflow pump was set to achieve the desired split between distillate and underflow, while the heat input to the column base was adjusted to keep the level in the column base constant.

Each step in the process was piloted sequentially. The organic feed was first fractionated to separate light billing acetonitrile from an array of heavy boilers, followed by the aqueous acetonitrile stream to purge large quantities of water from acetonitrile. The distillates from both steps were combined for the next stage distillation. The under flows from each distillation were removed to waste. The water content of the combined distillates was adjusted to a specific value by adding fresh water. The water acts as an entrainer in the next distillation step. The mixture was then distilled during the next phase to remove light boiling impurities overhead in the form of light boiling aqueous azeotropes, leaving a water/acetonitrile rich mixture to leave the column in the underflow.

The underflow is subsequently passed to the double-column dewatering system to produce an acetonitrile product with very low water content. Due to the upper limit of 980 mbars on the column, the design target of 2 to 3 bars for the second stage column could not be piloted. Instead, this column was operated at 980 mbars and the split between distillate and underflow adjusted to produce a reduced amount of dry acetonitrile. The first stage column was operated at 300 mbars.

Table 5.2 below shows the concentrations of the principal components in the organic and aqueous organic wastes in the same table. Only the main components that present a separation issue are shown. Many other components are present at small concentrations but are not an issue for separation.

TABLE 5.2
Feed compositions for organic and aqueous organic distillations

| | Organic | | Aqueous | |
|---|---|---|---|---|
| | Feed | Units | Feed | Units |
| Ethanol | 0 | [ppmW] | 3399.4 | [ppmW] |
| Propionitrile | 61.5 | [ppmW] | 9.2 | [ppmW] |
| Acrylonitrile | 28.3 | [ppmW] | 0 | [ppmW] |
| Ethylacetate | 0 | [ppmW] | | [ppmW] |
| Pyridine | 9271.5 | [ppmW] | 0 | [ppmW] |
| Toluene | 931.6 | [ppmW] | 0 | [ppmW] |
| 2,6 Lutidine | 26384.9 | [ppmW] | 0 | [ppmW] |
| Acetonitrile | 86.28 | [wt %] | 11.24 | [wt %] |
| Water (KF) | 510 | [ppmW] | 88.42 | [wt %] |
| Colour | brown | | Colourless | |

TABLE 5.3
Column configuration

| Parameter | Experiment | Units |
|---|---|---|
| Number of Stages | 30 | [—] |
| Reflux Ratio | 1.5 | [—] |
| Stages Rectification | 20 | [—] |
| Stages Stripping | 5 | [—] |
| Feed Temperature | 30 | [° C.] |
| Feed location | 6 to 8 | [Stage] |
| Distillate fraction | 90-91 | [wt %] |
| Underflow fraction | 10-9 | [wt %] |
| System Pressure | 200 | [mbars] |
| Head temperature | 36-38 | [° C.] |
| Underflow temperature | 52-53 | [° C.] |
| Feed rate | 2.5 | [kg/hr] |

TABLE 5.4
Stream compositions from organic waste distillation column

| | Distillate | | Underflow | |
|---|---|---|---|---|
| Ethanol | 0 | [ppmW] | | |
| Acrylonitrile | 2.3 | [ppmW] | | |
| Ethylacetate | 11.5 | [ppmW] | | |
| Pyridine | 0 | [ppmW] | 12.6 | [Area %] |
| Toluene | 871.5 | [ppmW] | | [Area %] |
| 2,6 Lutidine | 0.3 | [ppmW] | 45.4 | [Area %] |
| Acetonitrile | 99.91 | [wt % | 39 | [Area %] |
| Water | 500 | [ppmW] | 263 | [ppmW] |

Table 5.3 shows the configuration of the column to conduct the first stage organic waste stream distillation. Table 5.4 shows the composition of both the distillate and underflow streams from the distillation. The yield of acetonitrile in the distillate stream was approximately 95.5% and acetonitrile purity was 99.91% with the major impurity being toluene.

The column was subsequently set-up to distill acetonitrile from the aqueous organic stream. The feed stream is as given in table 5.2 and the distillate and underflow compositions are given in table 5.5. The column was operated as listed in table 5.6 at 450 mbars and at this pressure, the azeotropic composition of water and acetonitrile was distilled overhead. The yield of acetonitrile in the distillate stream is 99.99% which ethanol at a concentration of 2.69% is the dominant impurity.

TABLE 5.5
Distillate/underflow compositions
from the aqueous organic column

| | Distillate | | Underflow | |
|---|---|---|---|---|
| Ethanol | 2.69 | [wt %] | | |
| Acrylonitrile | 1.7 | [ppmW] | | |
| Propionitrile | 88 | [ppmW] | | |
| Pyridine | 1.1 | [ppmW] | | |
| Toluene | 0.2 | [ppmW] | | |
| 2,6 Lutidine | 1.8 | [ppmW] | | |
| Acetonitrile | 73.3 | [wt %] | 2400 | [ppmW] |
| Water | 24.04 | [wt %] | 99.76 | [wt %] |

TABLE 5.6
Column configuration

| | Experiment | Units |
|---|---|---|
| Number of Stages | 10 | [—] |
| Reflux Ratio | 0.2 | [—] |
| Stages Rectification | 5 | [—] |
| Stages Stripping | 5 | [—] |
| Feed Temperature | 35 | [° C.] |
| Feed location | 6 to 8 | [Stage] |
| Distillate fraction | 16 | [wt %] |
| Underflow fraction | 94 | [wt %] |
| System Pressure | 450 | [mbars] |
| Head temperature | 58-60 | [° C.] |
| Underflow temperature | 82-84 | [° C.] |
| Feed rate | 14 | [kg/hr] |

The distillate from the previous two distillations were combined to create the feed to the following distillation and is shown in table 5.7. Sufficient water which acts as an entrainer was added to reach a concentration of 16.17%. The column was operated at atmospheric pressure and according to the configuration shown in table 5.8. The composition of the distillate and underflow streams are shown in table 5.9. The distillate contains practically all the impurities in the feed stream. The only impurity in the underflow is ethanol present at 2.5 ppm and propionitrile at 20.7 ppm. The remaining is a mixture of acetonitrile and water. The yield of acetonitrile in the underflow stream is 90%. The acetonitrile has been purified at this stage in the process. It only remains to separate the water away from the acetonitrile to render the purified and dried acetonitrile product. This is accomplished by a series of two columns operating in series as described previously.

TABLE 5.7
Feed composition

| | Organic | |
|---|---|---|
| | Feed | Units |
| Ethanol | 4651 | [ppmW] |
| Propionitrile | 15.3 | [ppmW] |
| Acrylonitrile | 0 | [ppmW] |
| Ethylacetate | 38 | [ppmW] |
| Pyridine | 0 | [ppmW] |
| Toluene | 1205.2 | [ppmW] |
| 2,6 Lutidine | 0 | [ppmW] |
| Acetonitrile | 83.24 | [wt %] |
| Water | 16.17 | [wt %] |
| Colour | Clear | |

TABLE 5.8
Column configuration

| | Experiment | Units |
|---|---|---|
| Number of Stages | 30 | [—] |
| Reflux Ratio | 27 | [—] |
| Stages Rectification | 10 | [—] |
| Stages Stripping | 20 | [—] |
| Feed Temperature | 25 | [° C.] |
| Feed location | 14 to 16 | [Stage] |
| Distillate fraction | 9.7 | [wt %] |
| Underflow fraction | 88-89 | [wt %] |
| System Pressure | 950 | [mbars] |
| Head temperature | 74-75 | [° C.] |
| Underflow temperature | 77-78 | [° C.] |
| Feed rate | 3 to 5 | [kg/hr] |

TABLE 5.9
Distillate/underflow compositions from the aq organic column

| | Distillate | | Underflow | |
|---|---|---|---|---|
| Ethanol | 3.44 | [wt %] | 2.5 | [ppmW] |
| Propionitrile | 0 | [ppmW] | 20.7 | |
| Acrylonitrile | 14.3 | [ppmW] | 0 | |
| Ethylacetate | 225.1 | [ppmW] | 0.1 | |
| Pyridine | 0.2 | [ppmW] | 0 | |
| Toluene | 3813.1 | [ppmW] | 0 | |
| 2,6 Lutidine | 0 | [ppmW] | 0 | [ppmW] |
| Acetonitrile | 81.76 | [wt %] | 81.9 | [wt %] |
| Water | 14.4 | [wt %] | 18.1 | [wt %] |

The first column in the two-column series is expected to operate at 200 mbars or lower in an optimized process, but due to utility constraints was operated at 300 mbars in this example. The feed composition corresponds to the underflow composition from the previous stage distillation also shown in table 5.10. The column configuration is as shown in table 5.11. The distillate and underflow stream compositions are shown in table 5.12. There are only minor concentrations of impurities in the distillate stream (toluene, acrylonitrile, ethyl acetate, propionitrile). The yield of acetonitrile in the distillate stream is 99.99%.

TABLE 5.10
Feed composition

| | Organic | |
|---|---|---|
| | Feed | Units |
| Ethanol | 2.5 | [wt %] |
| Propionitrile | 20.7 | [ppmW] |
| Acrylonitrile | 0 | [ppmW] |
| Ethylacetate | 0.1 | [ppmW] |
| Pyridine | 0 | [ppmW] |
| Toluene | 0 | [ppmW] |
| 2,6 Lutidine | 0 | [ppmW] |
| Acetonitrile | 81.9 | [wt %] |
| Water | 18.1 | [wt %] |

TABLE 5.11
Column configuration

| | Experiment | Units |
|---|---|---|
| Number of Stages | 15 | [—] |
| Reflux Ratio | 0.1 | [—] |
| Stages Rectification | 5 | [—] |
| Stages Stripping | 10 | [—] |
| Feed Temperature | 25 | [° C.] |
| Feed location | 6 to 8 | [Stage] |
| Distillate fraction | 88-89 | [wt %] |
| Underflow fraction | 11-12 | [wt %] |
| System Pressure | 300 | [mbars] |
| Head temperature | 44-45 | [° C.] |
| Underflow temperature | 72-74 | [° C.] |
| Feed rate | 5.0 | [kg/hr] |

TABLE 5.12
Distillate/underflow compositions
from the aqueous organic column

| | Distillate | | Underflow | |
|---|---|---|---|---|
| Propionitrile | 26.2 | [ppmW] | 0 | [ppmW] |
| Ethanol | 9 | [ppmW] | 0 | [ppmW] |
| Acrylonitrile | 9 | [ppmW] | 0 | [ppmW] |
| Ethylacetate | 9 | [ppmW] | 0 | [ppmW] |
| Pyridine | 0 | [ppmW] | 0 | [ppmW] |
| Toluene | 2 | [ppmW] | 0 | [ppmW] |
| 2,6 Lutidine | 0 | [ppmW] | 0 | [ppmW] |
| Acetonitrile | 87.04 | [wt %] | 4.8 | [ppmW] |
| Water (KF) | 12.96 | [wt %] | 99.99 | [wt %] |

The second column in the two series dewatering section is expected to operate at a pressure in the 2 to 3 bar range, but since the column used in the test is limited to 1 bar, this was used as the operating pressure. The objective was to demonstrate that the distillate from the previous stage could be dewatered to a very high degree. The column was configured as shown in table 5.14 and the feed stream composition, derived from the previous stage is also shown in table 5.13. The resulting product (underflow) and distillate streams from this column are shown in table 5.15. The pyridine (5 ppm) in the product was not in the feed and is assumed to be a contaminant residue from previous distillation stages. The high concentration of propionitrile (142 ppm) is since all the propionitrile in the feed stream (26.2 ppm) ends up in the underflow but only 15% of the acetonitrile ends up there. A water content of 110 ppm was achieved. A further processing step such as an adsorbent may be required to reach below the 30-ppm specification value. A theoretical yield value of 99.99% can be assumed for this stage.

TABLE 5.13
Feed composition

| | Organic | |
|---|---|---|
| | Feed | Units |
| Propionitrile | 26.2 | [ppmW] |
| Ethanol | 9 | [ppmW] |
| Acrylonitrile | 9 | [ppmW] |
| Ethylacetate | 9 | [ppmW] |
| Pyridine | 0 | [ppmW] |
| Toluene | 2 | [ppmW] |
| 2,6 Lutidine | 0 | [ppmW] |
| Acetonitrile | 87.04 | [wt %] |
| Water (KF) | 12.96 | [wt %] |

TABLE 5.14
Column configuration

| | Experiment | Units |
|---|---|---|
| Number of Stages | 30 | [—] |
| Reflux Ratio | 0.25 | [—] |
| Stages Rectification | 10 | [—] |
| Stages Stripping | 20 | [—] |
| Feed Temperature | 25 | [° C.] |
| Feed location | 14 to 16 | [Stage] |
| Distillate fraction | 85 | [wt %] |
| Underflow fraction | 15 | [wt %] |
| System Pressure | 980 | [mbars] |
| Head temperature | 74-75 | [° C.] |
| Underflow temperature | 83-84 | [° C.] |
| Feed rate | 5.0 | [kg/hr] |

TABLE 5.15
Distillate/underflow compositions
from the aqueous organic column

| | Distillate | | Underflow | |
|---|---|---|---|---|
| Propionitrile | 11.9 | [ppmW] | 142 | [ppmW] |
| Ethanol | 4.7 | [ppmW] | 0 | [ppmW] |
| Acrylnitrile | 0 | [ppmW] | 0 | [ppmW] |
| Ethylacetate | 0 | [ppmW] | 0 | [ppmW] |
| Pyridine | 0 | [ppmW] | 5 | [ppmW] |
| Toluene | 0 | [ppmW] | 0 | [ppmW] |
| 2,6 Lutidine | 0 | [ppmW] | 0 | [ppmW] |
| ACN | 83.39 | [wt %] | 99.98 | [ppmW] |
| Water (KF) | 16.52 | [wt %] | 110 | [wt %] |

An overall process yield across the five distillation stages is approximately 85%.

1. A process for recovering and/or purifying acetonitrile from waste acetonitrile (105, 205, 301) generated during oligonucleotide synthesis, the process comprising the steps:
A*) introducing an organic waste feedstock (105, 205) comprising acetonitrile, a first set of organic impurities having a lower boiling temperature than acetonitrile and a second set of organic impurities having a boiling temperature greater than acetonitrile, into a distillation column (106, 206) and separating the acetonitrile and first set of organic impurities from the second set of organic impurities, the acetonitrile and first set of organic impurities being drawn as a vapour from said distillation column and condensed to produce a distillate (107, 207), the second set of organic impurities being produced as the second distillation column bottoms (108, 208);
B*) introducing the distillate (107, 207) into a watering zone (109, 209) to produce a water enriched acetonitrile stream (111, 210, 210a); and
C*) introducing the water enriched acetonitrile stream (111, 210, 210a) into a second distillation column (112, 212) and separating the first set of organic impurities from the acetonitrile, the acetonitrile being produced as the second distillation column bottoms (113, 213);
such that recovered and/or purified acetonitrile is obtained.

2. A process for recovering and/or purifying acetonitrile from waste acetonitrile (301) generated during oligonucleotide synthesis, the process comprising the steps:
A′) introducing an organic waste feedstock (301) comprising acetonitrile, a first set of organic impurities having a lower boiling temperature than acetonitrile and a second set of organic impurities having a boiling temperature greater than acetonitrile, into a first mixing zone (303), and enriching with water, to produce a water enriched acetonitrile stream (303a) comprising said impurities;
B′) introducing the water enriched acetonitrile stream (303a) into a first distillation column (304) and separating the acetonitrile and the first set of organic impurities having a lower boiling temperature than acetonitrile from the second set of organic impurities having a boiling temperature greater than acetonitrile, the acetonitrile and first set of organic impurities being drawn as a vapour from said first distillation column and condensed to produce a first distillate (306), the second set of organic impurities being produced as the first distillation column bottoms (305);
C′) introducing the first distillate (306) comprising the acetonitrile and first set of organic impurities from step B′), into a watering zone (307), and enriching with water to produce a water enriched acetonitrile stream (307a); and
D′) introducing the first distillate (306) into a second distillation column (308), and separating the acetonitrile from the first set of organic impurities, the acetonitrile being produced as the second distillation column bottoms (309);
such that recovered and/or purified acetonitrile is obtained.

3. A process for recovering and/or purifying acetonitrile from waste acetonitrile (301, 302) generated during oligonucleotide synthesis, the process comprising the steps:
A-) introducing an organic waste feedstock (301) comprising acetonitrile, a first set of organic impurities having a lower boiling temperature than acetonitrile and a second set of organic impurities having a boiling temperature greater than acetonitrile, and an aqueous waste feedstock (302) comprising acetonitrile, a first set of aqueous impurities having a lower boiling temperature than acetonitrile and a second set of aqueous impurities having a boiling temperature greater than acetonitrile, into a first mixing zone (303), and combining therein, to form an acetonitrile waste feedstock comprising said impurities;
B-) introducing the acetonitrile waste feedstock into a first distillation column (304) and separating the acetonitrile and the first set of organic and aqueous impurities having a lower boiling temperature than acetonitrile from the second set of organic and aqueous impurities having a boiling temperature greater than acetonitrile, the acetonitrile and first set of organic and aqueous impurities being drawn as a vapour from said first distillation column and condensed to produce a first distillate (306), the second set of organic and aqueous impurities being produced as the first distillation column bottoms (305);
C-) introducing the first distillate (306) comprising acetonitrile and first set of organic and aqueous impurities, into a watering zone (307) and enriching with water to produce a water enriched acetonitrile stream (307a);
D-) introducing the water enriched acetonitrile stream (307a) into a second distillation column (308) and separating the acetonitrile from the first set of organic and aqueous impurities, the acetonitrile being produced as the second distillation column bottoms (309);
such that recovered and/or purified acetonitrile is obtained.

4. The process according to claim 2, wherein the second distillation column bottoms (309) comprises an acetonitrile/water azeotrope.

5. The process according to claim 4, further comprising the step of
E′) introducing the second distillation column bottoms product (309) of step D′) or step D-) into a third distillation column (311) and separating the acetonitrile from the acetonitrile/water azeotrope, the acetonitrile being produced as the third distillation column bottoms (312).

6. The process according to claim 1, wherein water is added to the distillate (107) of step B* to produce a water enriched acetonitrile stream (111).

7. The process according to claim 1, wherein water is added to the distillate (207) of step B* to produce a water enriched acetonitrile stream (210).

8. The process according to claim 1, wherein the distillate (207) of step B* is combined with an acetonitrile stream comprising water to produce a water enriched acetonitrile stream (210a).

9. The process according to claim 8, wherein the acetonitrile stream comprising water is obtained from a distillation of an aqueous waste feedstock generated during oligonucleotide synthesis.

10. The process according to claim 9, wherein the distillation comprises the step of:
introducing an aqueous waste feedstock (201) comprising acetonitrile, a first set of aqueous impurities having a lower boiling temperature than acetonitrile and a second set of aqueous impurities having a boiling temperature greater than acetonitrile, into a distillation column (202) and separating the acetonitrile and first set of impurities from the second set of impurities, the acetonitrile and first set of impurities being drawn as a vapour from said distillation column and condensed to produce a distillate (203), the second set of impurities being produced as the distillation column bottoms (204).

11. The process according to claim 1 for recovering and/or purifying acetonitrile from waste acetonitrile (101, 105) generated during oligonucleotide synthesis, the process comprising the steps:
A1) introducing an aqueous waste feedstock (101) comprising acetonitrile, a first set of aqueous impurities having a lower boiling temperature than acetonitrile and a second set of aqueous impurities having a boiling temperature greater than acetonitrile, into a first distillation column (102) and separating the acetonitrile and first set of impurities from the second set of impurities, the acetonitrile and first set of impurities being drawn as a vapour from said first distillation column and condensed to produce a first distillate (103), the second set of impurities being produced as the first distillation column bottoms (104);
B1) introducing an organic waste feedstock (105) comprising acetonitrile, a first set of organic impurities having a lower boiling temperature than acetonitrile and a second set of organic impurities having a boiling temperature greater than acetonitrile, into a second distillation column (106) and separating the acetonitrile and first set of organic impurities from the second set of organic impurities, the acetonitrile and first set of organic impurities being drawn as a vapour from said second distillation column and condensed to produce a second distillate (107), the second set of organic impurities being produced as the second distillation column bottoms (108);
C1) introducing the second distillate (107) into a watering zone (109) and enriching with water to produce a water enriched acetonitrile stream (111);
D1) introducing the water enriched acetonitrile stream (111) into a third distillation column (112) and separating the first set of organic impurities from the acetonitrile, the acetonitrile being produced as the third distillation column bottoms (113); and
E1) feeding the first distillate (103) of step A1 and the third distillation column bottoms (113) of step D1 to a mixing zone (116) and combining therein, to produce an acetonitrile enriched stream (110);
such that recovered and/or purified acetonitrile is obtained.

12. The process according to claim 1 for recovering and/or purifying acetonitrile from waste acetonitrile (201, 205) generated during oligonucleotide synthesis, the process comprising the steps:
A) introducing an aqueous waste feedstock (201) comprising acetonitrile, a first set of aqueous impurities having a lower boiling temperature than acetonitrile and a second set of aqueous impurities having a boiling temperature greater than acetonitrile, into a first distillation column (202) and separating the acetonitrile and first set of impurities from the second set of impurities, the acetonitrile and first set of impurities being drawn as a vapour from said first distillation column and condensed to produce a first distillate (203), the second set of impurities being produced as the first distillation column bottoms (204);
B) introducing an organic waste feedstock (205) comprising acetonitrile, a first set of organic impurities having a lower boiling temperature than acetonitrile and a second set of organic impurities having a boiling temperature greater than acetonitrile, into a second distillation column (206) and separating the acetonitrile and first set of organic impurities from the second set of organic impurities, the acetonitrile and first set of organic impurities being drawn as a vapour from said second distillation column and condensed to produce a second distillate (207), the second set of organic impurities being produced as the second distillation column bottoms (208);
C) feeding the first distillate of step A (203) and the second distillate of step B (207) to a watering zone (209) and
C1) combining therein to produce a water enriched acetonitrile stream (210a), or
C2) combining therein and enriching with water to produce a water enriched acetonitrile stream (210); and
D) introducing the water enriched acetonitrile stream (210, 210a) into a third distillation column (212) and separating the first sets of impurities from the acetonitrile, the acetonitrile being produced as the third distillation column bottoms (213);
such that recovered and/or purified acetonitrile is obtained.

13. The process according to claim 11, wherein steps A1 and B1 or steps A and B are performed sequentially.

14. The process according to claim 11, wherein steps A1 and B1 or steps A and B are performed in parallel.

15. The process according to claim 1, wherein the distillation column of step A* operates in the range of 50 to 980 mbar, e.g., 100 to 300 mbar.

16. The process according to claim 1, wherein the distillation column of step C* operates in the range of 100 to 980 mbar, e.g., 900 to 980 mbar.

17. The process according to claim 11, wherein the distillation column of step B1 operates in the range of 50 to 980 mbar, e.g., 100 to 300 mbar.

18. The process according to claim 11, wherein the distillation column of step A1 operates in the range of 200 to 980 mbar, e.g., 400 to 500 mbar.

19. The process according to claim 11, wherein the distillation column of step D1 operates in the range of 100 to 980 mbar, e.g., 900 to 980 mbar.

20. The process according to claim 12, wherein the distillation column of step A operates in the range of 200 to 980 mbar, e.g., 400 to 500 mbar.

21. The process according to claim 12, wherein the distillation column of step B operates in the range of 50 to 980 mbar, e.g., 100 to 300 mbar.

22. The process according to claim 12, wherein the distillation column of step D operates in the range of 100 to 980 mbar, e.g., 900 to 980 mbar.

23. The process according to claim 1, wherein step C*, step C1 or step C further comprises mixing distillate (107, 207) with a recycled acetonitrile stream (122, 221) comprising an acetonitrile/water azeotrope and ethanol, wherein the recycled acetonitrile stream (122, 221) is a by-product of a further processing step.

24. The process according to claim 2, wherein step A′ further comprises mixing the water enriched acetonitrile stream (303a) with a recycled acetonitrile stream (316) comprising an acetonitrile/water azeotrope and ethanol, wherein the recycled acetonitrile stream (316) is a by-product of a further processing step.

25. The process according to claim 1, wherein the distillation column bottoms (113, 213, 309, 312) or the acetonitrile enriched stream (110) is further processed to reduce the water content of the acetonitrile to less than 30 parts per million.

26. The process according to claim 25, further comprising the step of introducing the distillation column bottoms (113, 213) or the acetonitrile enriched stream (110) into a fourth distillation column (117, 216), wherein the water content of the acetonitrile enriched stream is reduced by use of a pressure-swing distillation to recover acetonitrile having a water content of less than 30 parts per million.

27. The process according to claim 26, wherein the pressure-swing distillation comprises the steps:
F1) introducing the acetonitrile enriched stream (110) or the distillation column bottoms (113, 213) into a fourth distillation column (117, 216) and, performing a distillation at below atmospheric pressure to remove water therefrom, the acetonitrile being drawn as a vapour from said fourth distillation column and condensed to produce a fourth distillate (118, 217), and water being produced as the fourth distillation column bottoms (119, 218); and
F2) introducing the fourth distillate (118, 217) into a fifth distillation column (120, 219) and, performing a second distillation at above atmospheric pressure, e.g., 5 bar, to produce lower boiling fraction (122, 221) being drawn via the upper portion of the fifth distillation column (120, 219) and acetonitrile being produced as the fifth distillation column bottoms (121, 220).

28. The process according to claim 27, wherein the acetonitrile being produced as the fifth distillation column bottoms (121, 220) after the distillation of step F2 is passed over a water reducing adsorbent (124, 225), optionally wherein the water reducing adsorbent (124, 225) is molecular sieves.

29. The process according to claim 27, wherein the distillation of step F1 is performed in the range of 50 to 980 mbar, e.g., 50 to 200 mbar, e.g., 200 mbar.

30. The process according to claim 27, wherein the distillation of step F2 is performed in the range of 3 to 8 bar, e.g., 5 bar.

31. The process according to claim 25, wherein the recovered acetonitrile is passed over a water reducing adsorbent (124, 225).

32. The process according to claim 31, wherein the water reducing adsorbent (124, 225) is molecular sieves.

33. The process according to claim 1, wherein the recovered acetonitrile has a purity of at least 99.90% when measured by gas chromatography and a water content of less than 30 parts per million.

34. The process according to claim 27, further comprising recycling at least a portion of said lower boiling fraction (122, 221) from the fifth distillation column (120, 219).

35. The process according to claim 5, wherein the distillation of step B′ is performed at a lower pressure than the distillation of step D′, and the distillation of step D′ is performed at a lower pressure than step E′.

36. The process according to claim 2, wherein the distillation column of step B′ operates in the range of 50 to 980 mbar, e.g., 100 to 300 mbar.

37. The process according to claim 2, wherein the distillation column of step D′ operates in the range of 50 to 980 mbar, e.g., 100 to 300 mbar.

38. The process according to claim 5, wherein the distillation column of step E′ operates in the range of 50 to 980 mbar, e.g., 100 to 300 mbar.

39. The process according to claim 2, wherein the recovered acetonitrile (312) is passed over a water reducing adsorbent.

40. The process according to claim 39, wherein the water reducing adsorbent is molecular sieves.

41. The process according to claim 2, wherein the recovered acetonitrile has a purity of at least 99.90% when measured by gas chromatography and a water content of less than 30 parts per million.

42. The process according to claim 1, wherein the oligonucleotide synthesis is solid phase oligonucleotide synthesis.

43. A process for synthesizing an oligonucleotide and/or washing an oligonucleotide or support-bound oligonucleotide, the process comprising recovering and/or purifying acetonitrile from waste acetonitrile according to claim 1; and using at least a portion of the recovered and/or purified acetonitrile in a process for synthesizing an oligonucleotide and/or washing an oligonucleotide or support-bound oligonucleotide.

44. The process according to claim 43, wherein the oligonucleotide is RNA.

45. The process according to claim 43, wherein the oligonucleotide is DNA.

46. The process according to claim 43, wherein the oligonucleotide comprises both RNA and DNA.

47. The process according to claim 43, wherein the oligonucleotide is a modified oligonucleotide.

48. The process according to claim 47, wherein the modification is at the 2′ position of the sugar moiety and is selected from the group consisting of 2′-F, 2′-OMe, 2′-MOE, and 2′-amino, or wherein the oligonucleotide comprises a PMO, a LNA, a PNA, a BNA, or a SPIEGELMER.

49. The process according to claim 47, wherein the modification is in the nucleobase and is selected from the group consisting of a 5-methyl pyrimidine, a 7-deazaguanosine and an abasic nucleotide.

50. The process according to claim 47, wherein the modification is in the backbone and is selected from the group consisting of phosphorothioate, phosphoramidate and phosphorodiamidate.

51. The process according to claim 47, wherein the oligonucleotide is selected from an antisense oligonucleotide, an aptamer, siRNA, miRNA, and a gapmer.

52. The process according to claim 47, wherein the oligonucleotide is an antisense oligonucleotide.

53. The process according to claim 47, wherein the oligonucleotide is an siRNA.

54. The process according to claim 43, wherein the oligonucleotide is 10 to 200 nucleotides long.

55. The process according to claim 43, wherein the oligonucleotide is 18, 19, 20, 21, 22, 23, 24, 25, 26, 27, 28, 29 or 30 nucleotides long.

56. The process according to claim 43, wherein the oligonucleotide is an 18-mer, 19-mer, 20-mer, 21-mer, 22-mer, 23-mer, 24-mer, 25-mer, 26-mer, 27-mer, 28-mer, 29-mer or a 30-mer.

57. The process according to claim 43, wherein the oligonucleotide is a therapeutic oligonucleotide.

58. The process according to claim 43, wherein the oligonucleotide is a single stranded therapeutic oligonucleotide.

59. The process according to claim 43, wherein the oligonucleotide is a double stranded therapeutic oligonucleotide.

60. The process according to claim 43, wherein the oligonucleotide is inclisiran.

61. The process according to claim 43, wherein the oligonucleotide is pelacarsen.

62. The process according to claim 43, wherein the process for synthesizing an oligonucleotide comprises solid phase oligonucleotide synthesis.

63. A system for purifying and/or recovering acetonitrile from waste acetonitrile (105, 205, 301) generated during an oligonucleotide manufacturing process, the system comprising:
a first distillation column (106, 206, 304) configured to receive an acetonitrile organic waste stream (105, 205, 301) and produce a first distillate (107, 207, 306) comprising acetonitrile and a first set of organic impurities, the first distillation column (106, 206, 304) having a condenser connected to the upper portion of the first distillation column; and
a second distillation column (112, 212, 308) configured to receive the first distillate (107, 207, 306) and separate the first set of organic impurities from the acetonitrile, and produce purified acetonitrile as the second distillation column bottoms; wherein
the system further comprises a watering zone (109, 209, 303, 307) located such that
the first distillate (107, 207, 306) is first enriched with water before reaching the second distillation column (112, 212, 308), such that purified and/or recovered acetonitrile can be obtained; or
the waste acetonitrile (301) is enriched with water before reaching the first distillation column (304), such that purified and/or recovered acetonitrile can be obtained.

64. A system (100) for purifying and/or recovering acetonitrile from waste acetonitrile (101, 105) generated during an oligonucleotide manufacturing process, the system comprising:
a first distillation column (102) configured to receive an acetonitrile aqueous waste stream (101) and produce a first distillate (103) comprising acetonitrile and a first set of impurities, the first distillation column (102) having a condenser connected to the upper portion of the first distillation column (102);
a second distillation column (106) configured to receive an acetonitrile organic waste stream (105) and produce a second distillate (107) comprising acetonitrile and a first set of impurities, the second distillation column (106) having a condenser connected to the upper portion of the second distillation column (106); and
a third distillation column (112) configured to receive the second distillate (107) and separate the first set of organic impurities from the acetonitrile, and produce purified acetonitrile as the third distillation column bottoms; wherein
the system further comprises a watering zone (109) located such that the second distillate (107) is first enriched with water before reaching the third distillation column (112), such that purified and/or recovered acetonitrile can be obtained; and a mixing zone (116) configured to mix the first distillate (103) from the first distillation column (102) and the third distillation column bottoms (113) from the third distillation column (112).

65. A system (200) for purifying and/or recovering acetonitrile from waste acetonitrile (201, 205) generated during an oligonucleotide manufacturing process, the system comprising:
a first distillation column (202) configured to receive an acetonitrile aqueous waste stream (201) and produce a first distillate (203) comprising acetonitrile and a first set of impurities, the first distillation column (202) having a condenser connected to the upper portion of the first distillation column (202);
a second distillation column (206) configured to receive an acetonitrile organic waste stream (205) and produce a second distillate (207) comprising acetonitrile and a first set of impurities, the second distillation column (206) having a condenser connected to the upper portion of the second distillation column (206); and
a third distillation column (212) configured to receive the first and second distillates (203, 207) and separate the first sets of impurities from the first and second distillates, and produce purified acetonitrile as the third distillation column bottoms (213); wherein
the system further comprises a watering zone (209) located such that the first and second distillates (203, 207) are first mixed and enriched with water before reaching the third distillation column (212), such that purified and/or recovered acetonitrile can be obtained.

66. A system (300) for purifying and/or recovering acetonitrile from waste acetonitrile (301, 302) generated during an oligonucleotide manufacturing process, the system comprising:
a first distillation column (304) configured to receive a mixed acetonitrile aqueous and organic waste stream and produce a first acetonitrile distillate (306) comprising acetonitrile and a first set of impurities, the first distillation column (304) having a condenser connected to the upper portion of the first distillation column (304), e.g., connected to an overhead stream;
a second distillation column (308) configured to receive the first acetonitrile distillate (306) and produce a second acetonitrile distillate (309) comprising an acetonitrile/water azeotrope, the second distillation column (308) having a condenser connected to the upper portion of the second distillation column (308), e.g., connected to an overhead stream; and
a third distillation column (311) configured to receive the second acetonitrile distillate (309) and separate acetonitrile from the acetonitrile/water azeotrope, and produce acetonitrile as the third distillation column bottoms (312); wherein
the system further comprises a watering zone (303, 307) located such that the first acetonitrile distillate (306) is first enriched with water (307) before reaching the second distillation column (308), and/or
the organic waste acetonitrile (301) is first enriched with water by mixing an acetonitrile aqueous and organic waste stream (303) before reaching the first distillation column (304), such that purified and/or recovered acetonitrile can be obtained.

67. The system (100, 200, 300) according to claim 64, wherein the condenser of the first distillation column (102, 202, 304) is connected an overhead stream.

68. The system (100, 200, 300) according to claim 67, wherein the condenser of the second distillation column (106, 206, 308) is connected an overhead stream.

69. The system (100, 200) according to claim 68, further comprising
(i) at least a fourth distillation column (117, 216) configured to receive the third distillation column bottoms (113, 213) of the third distillation column (112, 212) and produce a fourth distillate (118, 217) comprising acetonitrile; and
(ii) a fifth distillation column (120, 219) configured to receive the fourth distillate (118, 217) from the fourth distillation column (117, 216), and produce a fifth distillation column bottoms (121, 220) product comprising highly pure acetonitrile.

70. The system (100, 200, 300) according to claim 69, further comprising a drying zone (124, 225), wherein the drying zone is configured to reduce the water content in the acetonitrile product to less than 30 parts per million.

71. The system (100) according to claim 64, substantially as shown in FIG. 1.

72. The system (200) according to claim 65, substantially as shown in FIG. 2.

73. The system (300) according to claim 66, substantially as shown in FIG. 3.

74. The system according to claim 63, wherein the waste acetonitrile is of industrial scale.
